(19) United States
(12) Patent Application Publication
(10) Pub. No.: US 2015/0165065 A1
(43) Pub. Date: Jun. 18, 2015

(54) NON-NATURAL MIC PROTEINS

(71) Applicant: AvidBiotics Corp., South San Francisco, CA (US)

(72) Inventors: Kyle LANDGRAF, Alameda, CA (US); Daniel P. Steiger, San Francisco, CA (US); Steven R. Williams, San Francisco, CA (US); David W. Martin, JR., Mill Valley, CA (US)

(73) Assignee: AvidBiotics Corp., South San Francisco, CA (US)

(21) Appl. No.: 14/562,534

(22) Filed: Dec. 5, 2014

Related U.S. Application Data
(63) Continuation-in-part of application No. 14/311,130, filed on Jun. 20, 2014.
Application No. 14/311,130 is a division of application No. 13/176,601, filed on Jul. 5, 2011, now Pat. No. 8,796,420.
Application No. 13/176,601 is a continuation-in-part of application No. 12/982,827, filed on Dec. 30, 2010, now Pat. No. 8,658,765.
(60) Provisional application No. 61/291,749, filed on Dec. 31, 2009.

(51) Int. Cl. A61K 47/48; A61K 39/00

(52) U.S. Cl. CPC A61K 47/48415; A61K 47/48246; A61K 39/0005; A61K 2039/55516

(57) ABSTRACT

This invention describes soluble, monovalent, non-natural protein molecules that can activate NK cells and certain T-cells to attack specific cellular target cells by attaching the NKG2D-binding portions of monovalent MICA or MICB protein, i.e. their α1-α2 platform domain, to the intended target cell specifically. The α1-α2 domain is contiguous with a heterologous α3 domain that has been genetically modified to bind directly or indirectly to the extracellular aspect of the target cell, thereby serving as the targeting domain. The genetic modification to create a non-natural and non-terminal targeting motif within the α3 domain can include a portion of an antibody, another protein molecule or portion thereof, a peptide, or a non-natural, modified α3 domain of a MIC protein.

### RELATED APPLICATIONS

This application is a continuation-in-part application of U.S. application Ser. No. 14/311,130, filed Jun. 20, 2014, which is a divisional application of U.S. Ser. No. 13/176,601, filed Jul. 5, 2011, now U.S. Pat. No. 8,796,420, which is a continuation-in-part application of U.S. application Ser. No. 12/982,827, filed Dec. 30, 2010, now U.S. Pat. No. 8,658,765, which claims priority from U.S. Provisional Application No. 61/291,749, filed Dec. 31, 2009, each of which is hereby incorporated by reference in its entirety.

### STATEMENT OF GOVERNMENT SUPPORT

This invention was made, in part, with government support under National Institutes of Health (NIH) Small Business Innovation Research (SBIR) grant number 1R43AI088979 awarded by the National Institute of Allergy and Infectious Diseases, and also, in part, from NCATS, NIH grant number R44TR001011. The government has certain rights in the invention.

### FIELD OF THE INVENTION

The instant invention relates generally to non-natural protein molecules that can recruit and activate NK cells, and more specifically to non-natural, monomeric, soluble, mammalian MHC class I chain-related (MIC) molecules modified within the α3 domain to contain a heterologous peptide that binds a target molecule on target cell.

### BACKGROUND OF THE INVENTION

Natural killer (NK) cells and certain (CD8+ αβ and γδ) T-cells of the immunity system have important roles in humans and other mammals as first-line, innate defense against neoplastic and virus-infected cells (Cerwenka, A., and L. L. Lanier. 2001. NK cells, viruses and cancer. Nat. Rev. Immunol. 1:41-49). NK cells and certain T-cells exhibit on their surfaces NKG2D, a prominent, homodimeric, surface immunoreceptor responsible for recognizing a target cell and activating the innate defense against the pathologic cell (Lanier, L L, 1998. NK cell receptors. Ann. Rev. Immunol. 16: 359-393; Houchins J P et al. 1991. DNA sequence analysis of NKG2, a family of related cDNA clones encoding type II integral membrane proteins on human NK cells. J. Exp. Med. 173: 1017-1020; Bauer, S et al., 1999. Activation of NK cells and T cells by NKG2D, a receptor for stress-inducible MICA. Science 285: 727-730). The human NKG2D molecule possesses a C-type lectin-like extracellular domain that binds to its cognate ligands, the 84% sequence identical or homologous, monomeric MICA [soluble form of MICA set forth in SEQ ID NOs: 1-6 and 13] and MICB [full protein of MICB set forth in SEQ ID NOs: 7-12], polymorphic analogs of the Major Histocompatibility Complex (MHC) Class I chain-related glycoproteins (MIC) (Weis et al. 1998. The C-type lectin superfamily of the immune system. Immunol. Rev. 163: 19-34; Bahram et al. 1994. A second lineage of mammalian MHC class I genes. PNAS 91:6259-6263; Bahram et al. 1996a. Nucleotide sequence of the human MHC class I MICA gene. Immunogentics 44: 80-81; Bahram and Spies T A. 1996. Nucleotide sequence of human MHC class I MICB cDNA. Immunogenetics 43: 230-233). Non-pathologic expression of MICA and MICB is restricted to intestinal epithelium, keratinocytes, endothelial cells and monocytes, but aberrant surface expression of these MIC proteins occurs in response to many types of cellular stress such as proliferation, oxidation and heat shock and marks the cell as pathologic (Groh et al. 1996. Cell stress-regulated human MHC class I gene expressed in GI epithelium. PNAS 93: 12445-12450; Groh et al. 1998. Recognition of stress-induced MHC molecules by intestinal γδT cells. Science 279: 1737-1740; Zwirner et al. 1999. Differential expression of MICA by endothelial cells, fibroblasts, keratinocytes and monocytes. Human Immunol. 60: 323-330). Pathologic expression of MIC proteins also seems involved in some autoimmune diseases (Ravetch, J V and Lanier L L. 2000. Immune Inhibitory Receptors. Science 290: 84-89; Burgess, S J. 2008. Immunol. Res. 40: 18-34). The differential regulation of NKG2D ligands, the polymorphic MICA (>50 alleles, see for examples SEQ ID NOS: 1-6 and 13 of FIG. 6) and MICB (>13 alleles, see for examples SEQ ID NOS: 7-12 of FIG. 6), is important to provide the immunity system with a means to identify and respond to a broad range of emergency cues while still protecting healthy cells from unwanted attack (Stephens H A, (2001) MICA and MICB genes: can the enigma of their polymorphism be resolved? Trends Immunol. 22: 378-85; Spies, T. 2008. Regulation of NKG2D ligands: a purposeful but delicate affair. Nature Immunol. 9: 1013-1015).

Viral infection is a common inducer of MIC protein expression and identifies the viral-infected cell for NK or T-cell attack (Groh et al. 1998; Groh et al. 2001. Co-stimulation of CD8+ αβT-cells by NKG2D via engagement by MIC induced on virus-infected cells. Nat. Immunol. 2: 255-260; Cerwenka, A., and L. L. Lanier. 2001). In fact, to avoid such an attack on its host cell, cytomegalovirus and other viruses have evolved mechanisms that prevent the expression of MIC proteins on the surface of the cell they infect in order to escape the wrath of the innate immunity system (Lodoen, M., K. Ogasawara, J. A. Hamerman, H. Arase, J. P. Houchins, E. S. Mocarski, and L. L. Lanier. 2003. NKG2D-mediated NK cell protection against cytomegalovirus is impaired by gp40 modulation of RAE-1 molecules. J. Exp. Med. 197:1245-1253; Stern-Ginossar et al., (2007) Host immune system gene targeting by viral miRNA. Science 317: 376-381; Stern-Ginossar et al., (2008) Human microRNAs regulate stress-induced immune responses mediated by the receptor NKG2D. Nature Immunology 9: 1065-73; Slavuljica, I A Busche, M Babic, M Mitrovic, I Ga\check{s}parovic, [symbol] Cekinovic, E Markova Car, E P Pugel, A Cikovic, V J Lisnic, W J Britt, U Koszinowski, M Messerle, A Krmpotic and S Jonjic. 2010. Recombinant mouse cytomegalovirus expressing a ligand for the NKG2D receptor is attenuated and has improved vaccine properties. J. Clin. Invest. 120: 4532-4545).

In spite of their stress, many malignant cells, such as those of lung cancer and glioblastoma brain cancer, also avoid the expression of MIC proteins and as a result may be particularly aggressive as they too escape the innate immunity system (Busche, A et al. 2006, NK cell mediated rejection of experimental human lung cancer by genetic over expression of MHC class I chain-related gene A. Human Gene Therapy 17: 135-146; Doubrovina, E S, M M Doubrovin, E Vider, R B Sisson, R J O'Reilly, B Dupont, and Y M Vyas, 2003. Evasion from NK Cell Immunity by MHC Class I Chain-Related Molecules Expressing Colon Adenocarcinoma (2003) J. Immunology 6891-99; Friese, M. et al. 2003. MICA/NKG2D-mediated immunogene therapy of experimental gliomas. Cancer Research 63: 8996-9006; Fuertes, M B, M V Girart, L L Molinero, C I Domaica, L E Rossi, M M Barrio, J Mordoh, G A Rabinovich and N W Zwirner. (2008) Intracellular Retention of the NKG2D Ligand MHC Class I Chain-Related Gene A in Human Melanomas Confers Immune Privilege and Prevents NK Cell-Mediated Cytotoxicity. J. Immunology, 180: 4606-4614).

### SUMMARY OF THE INVENTION

This invention describes soluble, monomeric, non-natural protein molecules that can recruit and activate NK cells and certain T-cells to attack specific cellular target cells by, after administration to a mammal, attaching the NKG2D-binding portions of MICA or MICB protein, i.e., their α1-α2 platform domain, specifically to the intended target molecule or molecules on the cellular target via a molecular targeting motif of the non-natural protein molecules of the invention.

Accordingly, in one aspect of the invention there are provided non-natural, monomeric, soluble, mammalian MHC class I chain-related (MIC) molecules containing an α1-α2 platform domain attached to a targeting motif, wherein the targeting motif contains a MIC α3 domain and one or more heterologous peptides, wherein the heterologous peptide(s) is/are inserted into one or more loops of the MIC α3 domain at a non-carboxy-terminal site, and wherein the heterologous peptides direct the binding of the targeting motif to a target molecule on a target cell, thereby delivering the attached α1-α2 platform domain to the target cell. In preferred embodiments, the heterologous peptide or peptides are inserted into the MIC α3 domain within one or more sites selected from loop 1, loop 2, and loop 3. In particular embodiments, loop 1 corresponds to amino acids numbers 190-199, loop 2 corresponds to amino acid residues 221-228, and loop 3 corresponds to amino acid residues 250-258 of the α3 domain of a MIC protein selected from the group consisting of SEQ ID NOs:1-13. In certain embodiments, the MIC molecule is glycosylated.

In some embodiments of the invention non-natural MIC proteins, the α1-α2 platform domain and the α3 domain are from a human MIC protein. In particular embodiments, the α1-α2 platform domain and the α3 domain are from a human MICA protein selected from the group consisting of SEQ ID NOs:1-6, and 13. In other embodiments, the α1-α2 platform domain and the α3 domain are from a human MICB protein selected from the group consisting of SEQ ID NOs:7-12. In preferred embodiments, the MICA or MICB protein is lacking its transmembrane domain.

In certain embodiments, the α3 domain of the non-natural MIC molecule is a complete native α3 domain without a deletion. In other embodiments, the α3 domain is a native α3 domain, wherein a portion of the domain has been deleted. In some embodiments, the portion deleted from the α3 domain is adjacent to the insertion site of the heterologous peptide. In particular embodiments, the portion deleted is within 10 amino acid residues of the insertion site. In other embodiments, the portion deleted is within 9, 8, 7, 6, 5, 4, 3, 2, or 1 amino acid residue of the insertion site. In other embodiments, the α3 domain comprises a deletion, insertion, amino acid substitution, mutation, or combination thereof at site different from the insertion site.

In particular embodiments of the non-natural MIC molecules, the insertion of the heterologous peptides are within one or more solvent-exposed loops of the α3 domain. In certain embodiments, a solvent-exposed loop corresponds to amino acids numbers 190-199, 208-211, 221-228, 231-240, 250-258, or 264-266 of the α3 domain within a MIC protein selected from the group consisting of SEQ ID NOs:1-13. In preferred embodiments, the insertion is in a solvent-exposed loop corresponding to amino acids numbers 190-199, 221-228, or 250-258 of the α3 domain within a MIC protein selected from the group consisting of SEQ ID NOs:1-13. In particular embodiments, all or a portion of one or more of loop 1, loop 2, or loop 3 is deleted and replaced with the heterologous peptide. In preferred embodiments, all of one or more of loop 1, loop 2, or loop 3 is deleted, and wherein further one, two, three, four, or five additional amino acids of the α3 domain adjacent to one or both sides of the deleted loop are deleted. In more preferred embodiments, all of one or more of loop 1, loop 2, or loop 3 is deleted, and wherein further one, two, or three additional amino acids of the α3 domain adjacent to one or both sides of the deleted loop are deleted. In some embodiments, a loop and two additional amino acids from both sides of the deleted loop are deleted, resulting in a deletion corresponding to amino acids residues 188-201, 219-230, or 248-260 of an α3 domain of a MIC protein selected from the group consisting of SEQ ID NOs:1-13. In a particular aspect, loop 1 is deleted and two additional amino acids from both sides of the deleted loop are deleted, corresponding to amino acids numbers 188-201 of an α3 domain of a MIC protein selected from the group consisting of SEQ ID NOs:1-13.

In some embodiments, more than one of loop 1, loop 2, or loop 3 of a MIC molecule contains a heterologous peptide. In some embodiments, the heterologous peptides bind to the same target molecule. In one aspect, the heterologous peptides contain the same amino acid sequence. In other embodiments, the heterologous peptides bind different target molecules.

In some embodiments of the invention, the target molecule is a cell-surface molecule. In particular embodiments, the cell-surface molecule is on the surface of a malignant cell or a virus infected cell. In particular embodiments in which the target cell is malignant, the target molecule is a human epidermal growth factor receptor 2 (HER2), NK-1R, epidermal growth factor receptor (EGFR), Erb2 or melanoma antigen; antigens of LNcaP and PC-3 cancer cells; a growth factor receptor, an angiogenic factor receptor, an integrin, CD3, CD19, CD20, CD113, CD271, or an oncogene-encoded protein product, or a fragment thereof. In preferred embodiments, the target molecule is selected from the group consisting of an integrin, ErbB2, FGF1 Receptor, FGF2 Receptor, FGF3 Receptor, IGF1 Receptor, IGF2 Receptor, VEGF1 Receptor, VEGF2 Receptor, CD19, CD20, CD113, CD271, or an oncogene-encoded protein product, or a fragment thereof. In some embodiments, the target molecule is an integrin. There are 18 known α-chains and 8 known β-chains forming at least 24 distinct integrin heterodimers, many of which are involved in pathogenic cells such as cancer cells (Koistinen and Heino, 2011. Integrins in Cancer Cell Invasion. Landes Bioscience NCBI Bookshelf ID NBK6070). Such integrins include α1β1, α2β1, α3β1, α4β1, α4β7, α5β1, α6β1, α6β4, α7β1, α8β1, α9β1, α10β1, αIIbβ1, αIIbβ3, αVβ1, αVβ3, αVβ5, αVβ6, and αVβ8. In preferred embodiments, the integrin is selected from the group consisting of αVβ3, αVβ5 and α5β1. In other embodiments, the target molecule is a growth factor receptor or a cell determinant (CD) protein. In preferred embodiments the growth factor receptor or CD protein is selected from the group consisting of ErbB2, FGF1-3 Receptors, IGF1 Receptor, IGF2 Receptor, VEGF1 Receptor, VEGF2 Receptor, CD19, CD20, CD113, and CD271.

In embodiments in which the target cell is infected by a virus, the target molecule on the target cell is a phosphotidylserine, or a phosphotidylserine with an accessory protein; or a surface glycoprotein encoded by a virus, an adenovirus, a human immunodeficiency virus, a herpetic virus, a pox virus, a flavivirus, a filovirus, a hepatitis virus, a papilloma virus, cytomegalovirus, vaccinia, rotavirus, influenza, a parvo virus, West Nile virus, rabies, polyoma, rubella, distemper virus, or Japanese encephalitis virus.

In another aspect of the invention, there are provided compositions containing the non-natural MIC molecules of the invention and a carrier or excipient.

In a further aspect of the invention, there are provided nucleic acid molecules encoding the non-natural, soluble, monomeric MIC molecules of the invention. In particular embodiments, there are provided nucleic acid molecules encoding non-natural, monomeric, soluble, mammalian MHC class I chain-related (MIC) molecules containing an α1-α2 platform domain attached to a targeting motif, wherein the targeting motif contains a MIC α3 domain and one or more heterologous peptides, wherein the heterologous peptide(s) is/are inserted into one or more loops of the MIC α3 domain at a non-carboxy-terminal site, and wherein the heterologous peptides direct the binding of the targeting motif to a target molecule on a target cell, thereby delivering the attached α1-α2 platform domain to the target cell. In particular embodiments of the nucleic acid molecules encoding non-natural MIC molecules, a polynucleotide encoding heterologous peptides are inserted within the nucleic acid sequence encoding a solvent-exposed loop of the α3 domain corresponding to amino acids numbers 190-199, 208-211, 221-228, 231-240, 250-258, or 264-266 of the α3 domain within a MIC protein selected from the group consisting of SEQ ID NOs:1-13. In preferred embodiments of the nucleic acid molecules encoding non-natural MIC molecules, the heterologous peptide or peptides are inserted into the MIC α3 domain within one or more sites selected from loop 1, loop 2, and loop 3. In particular embodiments, loop 1 corresponds to amino acids numbers 190-199, loop 2 corresponds to amino acid residues 221-228, and loop 3 corresponds to amino acid residues 250-258 of the α3 domain of a MIC protein selected from the group consisting of SEQ ID NOs:1-13.

In another aspect of the invention, there are provided libraries containing non-natural MIC molecules of the invention, in which the members of a library have diverse individual target binding properties.

In still another aspect of the invention, there are provided libraries containing genes encoding the non-natural MIC molecules of the invention, in which the members of a library have diverse individual target binding properties.

In still another aspect of the invention, there are provided methods of treating a mammal suspected of having a malignancy or viral infection by administering an effective amount of the a non-natural MIC molecule of the invention to the mammal, wherein the heterologous peptides direct binding of the targeting motif to the target molecule on a malignant cell or a virus-infected cell. In certain embodiments, the non-natural MIC molecule binds a NKG2D-bearing cell and a malignant cell or a NKG2D-bearing cell and a virus-infected cell, resulting in the adhesion of the NKG2D-bearing cell to the malignant cell or the virus-infected cell. In particular embodiments, the adhering NKG2D-bearing cell destroys the viability of the malignant cell or the virus-infected cell.

### BRIEF DESCRIPTION OF THE DRAWINGS

FIG. 1 shows the structure of the soluble form of human MICA. The human MICA structure represented as ribbons as solved by Pingwei Li, et al. (Nature Immunology 2, 443-451, 2001). The α1 and α2 domains provide the binding sites for the NKG2D homodimer. The α3 domain is a member of the Ig super-family and in this soluble form expressed in E. coli contains the C-terminus.

FIG. 2 shows a photograph of the SDS-PAGE analysis of the cytosolic (“cytosol”), cytoplasmic membrane (“Cytopl memb”), and outer membrane (“Outer memb”) proteins from induced (lanes labeled “B”) or un-induced (lanes labeled “A”) cultures of E. coli harboring pKK29 detected by Coomassie blue staining or Western blotting with an antibody against human MICA. The molecular weights are indicated. The fusion of MICA α3 domain to intimin (EaeA) is expressed on the outer membrane of the E. coli cells induced with arabinose.

FIG. 3 is a cartoon of the configuration of DNA encoding soluble MICA amino acids 1-276 in the mammalian expression vector, pc5DNA/FRT. The positions of CMV promoter, secretion signal, His-hexamer tag (“His6”; SEQ ID NO:127), “tight” loop 1 (amino acids 188-201) and loop 3 (amino acids 250-258) of α3, and the polyA tail of bgh are shown.

FIG. 4 shows the configuration of MICA α3-encoding DNA fused to DNA encoding a portion of M13 phage pIII. The positions of the lac promoter, P_{lac}, pIII secretion signal, a FLAG tag and “tight” loop 1 (amino acids 188-201) and loop 3 (amino acids 250-258) of α3 are shown.

FIG. 5 shows a schematic of a DGR-based approach for diversifying the α3 domain of human MICA.

FIG. 6 shows a schematic of the structures of EaeA and the EaeA-α3 fusion proteins displayed on the surface of E. coli. OM is outer membrane; D1-D3 are Ig superfamily motifs, Xa is a factor X cleavage site, and H6 is a hexa-histadine.

FIGS. 7A through 7J provide the amino acid or nucleic acid sequences for SEQ ID NOs: 1-126.

FIG. 8 shows a structure-directed mutagenesis of the α1-α2 domain of MICA for enhanced NKG2D affinity. (A) Structure of the α1-α2 domain of MICA (PDB 1HYR) with the NKG2D-binding surface mapped to 57 residues colored dark grey. (B) Six positions were identified as key sites for NKG2D affinity mutations. The wild-type amino acid residues are labeled and their side chains shown in dark grey spheres.

FIG. 9 shows NKG2D-Fc competition ELISAs to affinity rank α1-α2 variants. (A) Titration data for a panel of α1-α2 affinity variants (15-18), wild-type (WT), or WED soluble MICA proteins inhibiting human NKG2D-Fc binding to plate-coated MICA. (B) The same set of proteins in (A) titrated against mouse NKG2D-Fc. In both assays variants 15, 16, 17, and 18 display IC₅₀ values significantly less than both WT and WED proteins. The equilibrium IC₅₀ values are shown in Table 8.

FIG. 10 is an analysis of the association and dissociation kinetics for α1-α2 variants binding to NKG2D. Kinetic traces for a panel of α1-α2 variants. The association and dissociation phases were fit using a single exponential 1:1 binding equation and on- and off-rate constants derived from the fits are shown in Table 8.

FIG. 11 is NK-mediated target cell killing assay for the α1-α2 variants targeting FGFR3-expressing target cells. NKL effector cells were co-incubated with calcein-loaded, FGFR3-expressing P815 target cells at an effector:target ratio of 15:1. Increasing concentrations of a negative control MICA (sMICA) had no effect on target cell lysis, whereas the indicated α1-α2 variants stimulated target cell lysis. Relative to WT, variants 16, 17, and 18 exhibited significantly increased killing at low concentrations.

FIG. 12 is a diagram of an insertable variable fragment, iFv. (A) Structure of variable light (VL) and variable heavy (VH) domains from FGFR3-binding antibody showing the domain topology of the iFv format. Grey arrows represent the 2 linker regions (LR), one and only one of which is used traditionally to connect the termini of VL and VH to create an scFv. The LR with a dotted border connected the C-terminus of VL to the N-terminus of VH (visible behind the molecule). The LR with a solid border connected the C-terminus of VH to the N-terminus of VL. The split in the VL domain between strand 1 (S1) and strand 2 (S2) created the new non-natural N- and C-termini (Nt and Ct) as described in text. As a result of this split, the VL has been divided into an N-terminal segment (VLN) and a C-terminal segment (VLC). The 6 CDRs of VL and VH are represented as the loops at the top of the figure. (B) Scheme of the domain layout for inserting an iFv into loop 1 (L1) of MICA-α3 with or without a spacer region (SR). An iFv could also be similarly inserted into loop 2 (L2) and/or loop 3 (L3).

FIG. 13 Titration curves for the modified sMICA molecules binding to FGFR3 coated wells. Bound sMICA was detected by ELISA using NKG2D-Fc to confirm the bispecific binding activity. Both versions of the inserted variable fragments (MICA-α3-iFv.1 and MICA-α3-iFv.2) bound FGFR3 comparably to the C-terminal fusion of a scFv (MICA-scFv).

FIG. 14 shows the thermal stability of MICA-α3-iFv.2. ELISA titration curves of MICA-scFv (A) or MICA-α3-iFv.2 (B) binding to FGFR3 coated wells after exposure to the indicated temperatures (degrees Celsius) for 1 hour. The MICA-α3-iFv.2 exhibited strong binding to FGFR3 after exposure to 80° C., whereas MICA-scFv lost significant activity after exposure to 70° C.

FIG. 15 shows NK-mediated target cell lysis assays. NKL effector cells were co-incubated with calcein-loaded, FGFR3-expressing P815 target cells at an effector:target ratio of 15:1. Increasing concentrations of a negative control MICA (sMICA) had no effect on target cell lysis, whereas the indicated FGFR3-binding MICA variants stimulated target cell lysis. Compared to MICA-scFv, both MICA-α3-iFv variants directed greater target cell lysis.

FIG. 16 shows target binding and cell lysis activity of a CD20-specific sMICA variant. MICA-α3-iFv.3 exhibits titratable binding to CD20 coated wells in an ELISA (A), and also enhances NK-mediated cell lysis of CD20-expressing Ramos cells (B). In (B), NKL effector cells were co-incubated with calcein-loaded CD20-expressing Ramos target cells at a effector:target ratio of 15:1, and increasing concentrations of either the negative control (sMICA) or MICA-α3-iFv.3.

FIGS. 17A through 17F provide the amino acid or nucleic acid sequences for SEQ ID NOs: 127-143.

### DETAILED DESCRIPTION OF THE INVENTION

This invention describes soluble, monomeric, non-natural MIC protein molecules that can recruit and activate NK cells and certain T-cells to attack specific cellular target cells by, after administration to a mammal, binding of the NKG2D-binding portions of MICA or MICB protein, i.e. their α1-α2 platform domain (amino acids 1-85 and 86-178, for the α1 domain and the α2 domain, respectively), to the intended target molecule or molecules specifically via a targeting motif attached to α1-α2 platform domain. The targeting motif includes an α3 domain of a MICA or MICB protein and inserted heterologous peptide or peptides that bind a target molecule.

A “heterologous peptide” is a peptide that is not naturally or normally within the α3 domain. In some embodiments, the heterologous peptide is integral to one of the solvent-exposed loops of the soluble MICA or MICB α3 domain. An integral heterologous peptide can be a non-terminal component of the MIC α3 domain and direct the binding of the MICα3 domain to a target molecule. A heterologous peptide may be inserted into a α3 domain loop between two adjacent residues of the loop without deleting any of the existing loop. Alternatively, all or a portion of the loop may be deleted and replaced with the heterologous peptide. In preferred embodiments, all of the loop is deleted and replaced with the insert. In additional embodiments, all of the loop is deleted and one, two, three, four, or five additional amino acids of the α3 domain adjacent to one or both sides of the deletion site are also deleted. In preferred embodiments one, two or three residues from one or both sides of the deletion site are deleted. In certain aspects, residues corresponding to 188-201 and/or residues 250-258 of SEQ ID NOs:1-13 are deleted. In some embodiments, a portion of the loop containing one or more residues is deleted and replaced with the heterologous peptide. In certain embodiments, the portion deleted is one to three residues of the loop, or one to five amino acid residues of the loop, or even one to seven residues of the loop. In particular embodiments, the portion deleted is within 10 amino acid residues of the insertion site. In other embodiments, the portion deleted is within 9, 8, 7, 6, 5, 4, 3, 2, or 1 amino acid residue of the insertion site.

In some embodiments, the heterologous peptide may include a spacer and a binding motif. Such spacers can be a short, flexible linker peptide used to position the binding motif of the heterologous peptide so that it may bind or improve its ability to bind its target molecule.

In particular embodiments, the heterologous peptide can include a portion of a complement-determining region of a natural or recombinant antibody, another protein or peptide molecule or binding motif. In certain embodiments, the heterologous peptide is a complement-determining region of an antibody. In other embodiments, the heterologous peptide further contains an attached polysaccharide or other carbohydrate, a nucleic acid molecule such as an aptamer or synthetic analog of a nucleic acid molecule. The incorporation of a heterologous peptide or peptides results in an unnatural (or non-natural), modified or converted α3 domain of a MICA or MICB protein, which acquires the useful function of directing the targeting the α1-α2 platform based on the binding properties (e.g., cognate binding partner) of the heterologous peptide or peptides. The non-natural, monovalent molecules of the invention have the distinct advantage of not being linked or restricted to a common presenting surface and thereby can be modified, formulated and administered to a mammal as traditional biopharmaceuticals.

In preferred embodiments, more than one of loop 1, loop 2, or loop3 of the non-natural, soluble, monomeric MIC proteins of the invention contain a heterologous peptide. In some embodiments, the heterologous peptides bind to the same target molecule. In one aspect, the heterologous peptides contain the same amino acid sequence within the binding motif. In some aspects, the target molecules may be on the same cell or the same cell type. In other aspects, the target molecules may be on different cells or cell types. In other embodiments, the heterologous peptides bind different target molecules. In some aspects, the target molecules may be on the same cell or the same cell type. In other aspects, the target molecules may be on different cells or cell types.

The modifications to the α3 domain desired include those that add or increase the specificity or sensitivity of the binding of the α3 domain to a target molecule, such as a molecule on the surface of a target cell, for example, a malignant cell or virus-infected cell. The α1-α2 platform domain is tethered to the modified targeting α3 domain and is diffusible in the intercellular or intravascular space of the mammal. Preferably the α1-α2 platform domains of the non-natural MIC proteins of the invention are at least 80% identical or homologous to a native or natural α1-α2 domain of a human MICA or MICB protein and bind an NKG2D receptor. In some embodiments, the α1-α2 platform domain is 85% identical to a native or natural α1-α2 platform domain of a human MICA or MICB protein and binds an NKG2D receptor. In other embodiments, the α1-α2 platform domain is 90%, 95%, 96%, 97%, 98%, or 99% identical to a native or natural α1-α2 platform domain of a human MICA or MICB protein and binds an NKG2D receptor. In some embodiments, the α3 domain is 85% identical (not including any modified loops) to a native or natural α3 domain of a human MICA or MICB protein. In other embodiments, the α3 domain is 90%, 95%, 96%, 97%, 98%, or 99% identical (not including any modified loops) to a native or natural α3 domain of a human MICA or MICB protein. Exemplary human MICA proteins (soluble form) include SEQ ID NOs: 1-6 and 13. Exemplary human MICB proteins (full protein) include SEQ ID NOs: 7-12.

In other embodiments, a heterologous peptide tag may be fused to the N-terminus or C-terminus of the soluble MIC protein to aid in the purification of the soluble MIC protein. Tag sequences include peptides such as a poly-histidine, myc-peptide or a FLAG tag. Such tags may be removed after isolation of the MIC molecule by methods known to one skilled in the art.

As used herein, a “soluble MIC protein”, “soluble MICA” and “soluble MICB” refer to a MIC protein containing the α1, α2, and α3 domains of the MIC protein but without the transmembrane or intracellular domains. Exemplary soluble MIC proteins include amino acid residues 1-274 or 1-276 of SEQ ID NOs:1-13.

As used herein, the “full MIC protein” refers to a MIC protein containing the α1, α2, and α3 domains, the transmembrane domain, and the intracellular domain. Exemplary full MIC proteins are set forth in SEQ ID NOs:7-12.

The invention further provides a library of MIC genes or resulting soluble MIC proteins, wherein each member of the library has or exhibits a different property, such as its binding property for the target cell, resulting in a library of diverse molecules. For example, the library can contain diverse individual target binding properties representing 10 or more different binding specificities. As used herein, “diverse individual target binding properties” refers to a library of MIC proteins, in which the individual members of the library bind to a different target molecule or have different affinities or avidities for the same target molecule. In some libraries of MIC proteins, two or more members may bind the same target but may have different binding affinities or avidities.

In a further embodiment of the invention, a mammal having a malignancy or a viral infection can be treated by administering an effective amount of the soluble MIC protein to affect the malignant or viral condition. The administration of the molecule to the mammal may result in the adhesion of NKG2D-bearing NK cells or T-cells to the target malignant or virus-infected cell, wherein the NK cell or T-cell destructively attacks or destroys the target malignant or virus-infected cell. The term “destroys” as used herein in the context of the invention methods means to destroy the viability of the target cell.

As used herein, “malignancy” or “malignant conditions” refer to cancer, a class of diseases in which a group of cells display uncontrolled growth, invasion that intrudes upon and destroys adjacent tissues, and sometimes metastasis (i.e., spreading to other locations in the body via lymph or blood). In some embodiments, the malignancy is a leukemia, a lymphoma, or a myeloma. In particular embodiments, the leukemia is acute lymphoblastic (ALL) leukemia, acute myelogenous leukemia (AML), chronic lymphocytic leukemia (CLL), chronic myelogenous leukemia (CML), hairy cell leukemia, or acute monocytic leukemia (AMOL); the lymphoma is Hodgkin's lymphoma or non-Hodgkin's lymphoma; and the myeloma is multiple myeloma. In other embodiments, the malignancy is a malignant solid tumor, including breast cancer, ovarian cancer, lung cancer, prostate cancer, pancreatic cancer, brain cancer, glioblastoma, head and neck cancer, colon cancer, esophageal cancer, liver cancer, stomach cancer, uterine cancer, endocrine cancer, renal cancer, bladder cancer, or cervical cancer. In preferred embodiments, the malignancy is breast cancer, ovarian cancer, lung cancer, prostate cancer, pancreatic cancer, brain cancer, glioblastoma, head and neck cancer, or colon cancer. In more preferred embodiments, the malignancy is breast cancer.

The invention also includes the means of converting the α3 domain (for example amino acids 182-274, in SEQ ID NOs: 1-13) of a MIC protein into a specific targeting domain that can directly deliver from the intercellular space its tethered α1-α2 domain to the target cell surface in order to attract, recruit or bind the NKG2D-bearing NK cell or T-cell.

Applications of these “passive vaccines” are to destroy pathologic cells that, in spite of being pathologic, do not express the appropriate level of ligands, such as MICA or MICB, that are necessary to attract NK cells or certain T-cells. For example, only 30% of human lung cancers express MICA (Busche, A et al. 2006). Glioblastoma cells over express an NK cell inhibitory signal that prevents innate immunity attack; however, over expressing the natural MICA gene product in lung cancer or glioblastoma cells in experimental animals, restores effective NK cell attack on the cancer (Friese, M. et al. 2003).

The high resolution structure of human MICA bound to the NKG2D receptor has been solved and demonstrates that the α3 domain of MICA has no direct interaction with the NKG2D receptor (Li et al. 2001. Complex structure of the activating immunoreceptor NKG2D and its MHC class I-like ligand MICA. Nature Immunol. 2: 443-451; Protein Data Bank accession code 1HYR). The α3 domain of MICA, like that of MICB, is connected to the α1-α2 platform domain by a short, flexible linker peptide, amino acids 175-182 [of SEQ ID 1-13], and itself is positioned naturally as “spacer” between the platform and the surface of the MIC expressing cell. The 3-dimensional structures of the human MICA and MICB α3 domains are nearly identical (root-mean square distance <1 Å on 94 C-αα's) and functionally interchangeable (Holmes et al. 2001. Structural Studies of Allelic Diversity of the MHC Class I Homolog MICB, a Stress-Inducible Ligand for the Activating Immunoreceptor NKG2D. J Immunol. 169: 1395-1400).

Furthermore, the 3-dimensional structures of the MIC proteins' Ig-like α3 domains resemble that of Tendamistat, and in a sequence inverted form, that of the human tenth fibronectin domain III; both structures have served as scaffolds for engineering protein binding motifs (Pflugrath, J W, G Wiegand, R Huber, L Vértesy (1986) Crystal structure determination, refinement and the molecular model of the α-amylase inhibitor Hoe-467A. J. Molec. Biol. 189: 383-386; Koide A, Bailey C W, Huang X, Koide S. 1998. The fibronectin type III domain as a scaffold for novel binding proteins. J. Mol. Biol. 284: 1141-1151; Li, R, RH Hoess, J S Bennett and WF DeGrado (2003) Use of phage display to probe the evolution of binding specificity and affinity in integrins. Protein Engineering 16: 65-72; Lipovsek, D. et al. (2007) Evolution of an inter-loop disulfide bond in high-affinity antibody mimics based on fibronectin type III domain and selected by yeast surface display: molecular convergence with single-domain camelid and shark antibodies. J. Mol Biol 368: 1024-1041; U.S. Pat. No. 7,153,661; Protein Data Bank accession code 1TTG).

One aspect of the invention contemplates engineering specific binding properties into 1 or more of the 6 solvent-exposed loops of the α3 domain of MICA or MICB, a soluble, non-natural MIC molecule is created that after administration to a mammal can diffuse in the intravascular or intercellular space and subsequently attach with high sensitivity and specificity to a target molecule on an intended target cell and, thereby promote binding and subsequent destructive attack of the particular target cell by NKG2D-bearing NK and/or T-cells. Examples of surface accessible molecules on target malignant cells include integrins, oncogene products or fragments thereof, such as NK-1R, human epidermal growth factor 2 (Her2 or ErbB2), growth factor receptors such as Epidermal Growth Factor Receptor (EGFR), FGF Receptor3, CD30, CD19, CD20, angiogenic factor receptors such as those for vascular endothelial growth factor (VEGF) receptor and VEGF-related molecules, melanoma antigens, and antigens of LNcaP and PC-3 prostate cancer cells. The surface accessible molecules on target virus-infected cells include “inside-out” phosphotidylserine with or without accessory proteins such as apolipoprotein H, Gash, MFG-E8; virus-encoded antigens, virus-encoded antigens of hepatitis viruses; adenoviruses; cytomegalovirus; other herpetic viruses; HIV especially p17; vaccinia; pox viruses; rotavirus; influenza; parvo viruses; West Nile virus; rabies; polyoma; papilloma viruses; rubella; distemper virus; and Japanese encephalitis virus (Balasubramanian, K and Schroit, A J. 2003. Ann. Rev. Physiol. 65: 701-734; Soares, M M, S W King & P E Thorpe. (2008) Targeting inside-out phosphatidylserine as a therapeutic strategy for viral diseases. Nature Medicine 14: 1358-62; Slavuljica et al., 2010). The present compositions can be produced by introducing specific binding motifs into the α3 domain of MICA or MICB deploying synthetic DNA, bacteriophage display or yeast or bacterial surface display technology, several of which have been deployed to create specific binding properties in Tendamistat and the human tenth fibronectin domain III (McConnell, S J and R H Hoess, (1995) Tendamistat as a Scaffold for Conformationally Constrained Phage Peptide Libraries. J. Molec. Biol 250: 460-470; Li et al. (2003); Sidhu, S. S. & S. Koide (2007) Phage display for engineering and analyzing protein interaction interfaces. Current Opinion in Struct. Biol. 17: 481-487; Lipovsek, D. et al. 2007). These methods involve making a library of α3 domain structures that are highly diversified within their solvent-exposed loops and from which to isolate the genotypes encoding those α3 domains that exhibit the desired phenotypic binding properties by selection, screening or panning, all well known to those ordinarily skilled in the art.

The diversity generating retroelements (DGR) of Miller et al. is an example of a method of generating diversity at desired amino acid positions within the loops (Medhekar, B. & J. F. Miller. 2007. Diversity-Generating Retroelements. Current Opinion in Microbiol. 10: 388-395 and U.S. Pat. No. 7,585,957). Because the α3 domains of human MICA and MICB are comprised of about 95 amino acids (182-276) of the 276 amino acid water-soluble form, all solvent-exposed loops, for example amino acids 190-199, 208-211, 221-228, 231-240, 250-258, or 264-266 of SEQ ID NOs: 1-13, can be diversified and even expanded with inserted amino acids by homing mutagenesis deploying a synthetic Template Repeat (TR) of a length not exceeding 200 nucleotides, a length known to be operable (Guo, H et al. 2008. Diversity-Generating Retroelement Homing Regenerates Target Sequences for Repeated Rounds of Codon Rewriting and Protein Diversification. Molecular Cell 31, 813-823).

Several factors guide the creation of the DGR-based library of diversified, solvent-exposed loops of the α3 domain. First, DGRs generate diversity in defined segments of protein-encoding DNA sequences, designated as variable repeats (VRs). For some heterologous sequences to function as VRs, they are flanked at their ends by initiation of mutagenic homing (IMH) sequences. The IMH sequences serve as cis-acting sites that direct mutagenic homing and determine the 3′ boundary of sequence diversification. Second, the 5′ boundary of VR diversification may be determined by the extent of homology between VR and its cognate TR. Only partial homology is required and mismatches are tolerated. Third, specific sites in VR which are subject to diversification may be determined by the location of adenine residues in TR. By inserting adenine residues at appropriate locations within “synthetic” TRs, specific VR-encoded amino acid residues can be diversified. Fourth, the atd protein, the TR-encoded RNA intermediate, and the RT reverse transcriptase efficiently function in trans when expressed on a plasmid vector, pDGR, under the control of a heterologous promoter, for example, P_{tetA} or P_{bad}. This provides a convenient means for turning on and off diversification within a bacterial cell and convenient access to the synthetic TR sequences to program the precise sites to be diversified. Furthermore, high level expression of trans-acting components results in highly efficient diversification.

A general outline of the DGR-based approach for diversifying the α3 domain is shown in FIG. 5. The sequences to be diversified correspond to the loops of α3 domain. An IMH sequence is positioned immediately downstream from the stop codon (about AV277) of the gene encoding α3 domain, creating a “synthetic VR” which will be subject to diversification.

The synthetic VR encoding the α3 domain will be diversified by the synthetic TR on plasmid pDGR (FIG. 5). This TR element includes an IMH* and upstream sequences that are homologous to VR. The specific VR residues that will be subject to mutagenesis are precisely programmed by the placement of adenines in TR, and high densities of adenine residues can be tolerated by the system. The pDGR also includes loci which encode Atd and the RT reverse transcriptase. Atd, TR and rt are expressed from the tightly regulated tetA promoter/operator (P_{tetA}), which allows precise control over the diversification process by the addition or removal of anhydotetracycline.

It is instructive to consider diversifying the α3 domain via the DGR mechanism in a standard phage display format. In this case, the α3 domain is fused to a filamentous phage coat protein encoded on a phagemid vector in E. coli. VR would include solvent-exposed loops of the α3 domain, and pDGR would be designed to efficiently diversify VR at specified locations within those loops (Guo et al. 2008). Activating atd, TR, and rt expression would mutagenize VR sequences present on phagemid genomes. This would result in the creation of a library of phage, each of which presents a diversified binding protein on its surface and packages the encoding DNA. Desired specificities would be selected by binding phage to the immobilized target molecule, for example the surface exposed protein product of oncogene Her2, washing to remove nonbinding phage, and reamplification and enrichment. Further rounds of optimization of the selected phenotype could be efficiently accomplished by simply infecting E. coli containing pDGR with the selected or panned phage and repeating the steps described above. This system is capable of generating library sizes that are several orders of magnitude greater than those achieved by conventional approaches. Of equal advantage is the extraordinary ease with which successive rounds of optimization may be achieved with cumulative improvements, but without compromise of the integrity of the α3 domain scaffold.

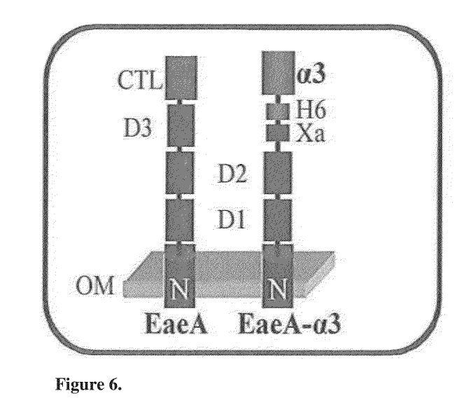

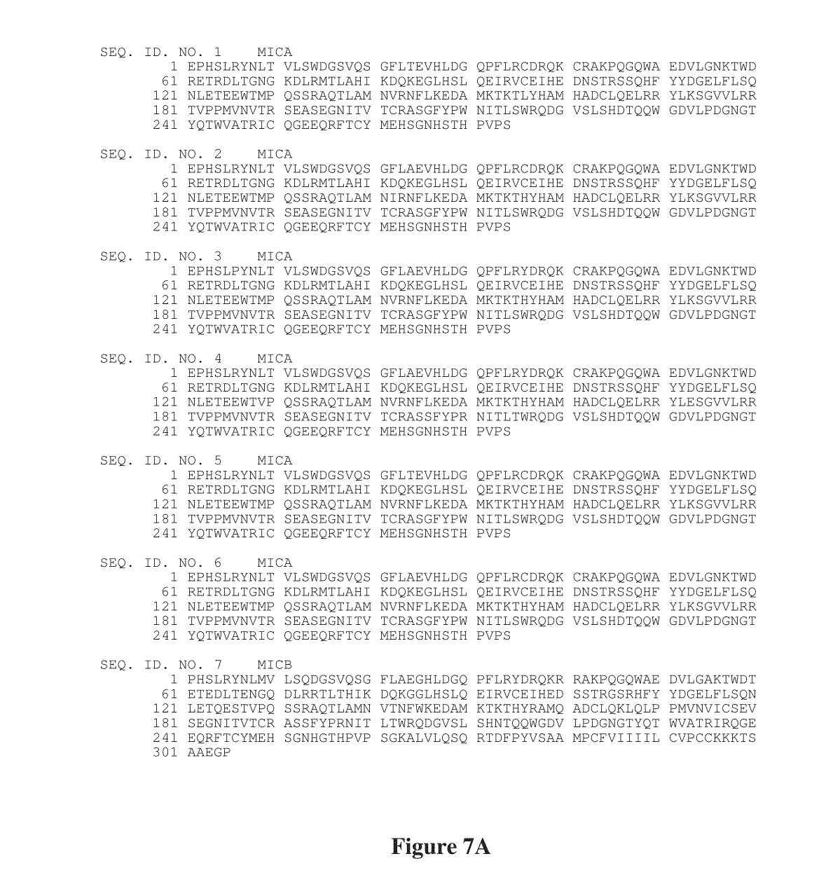

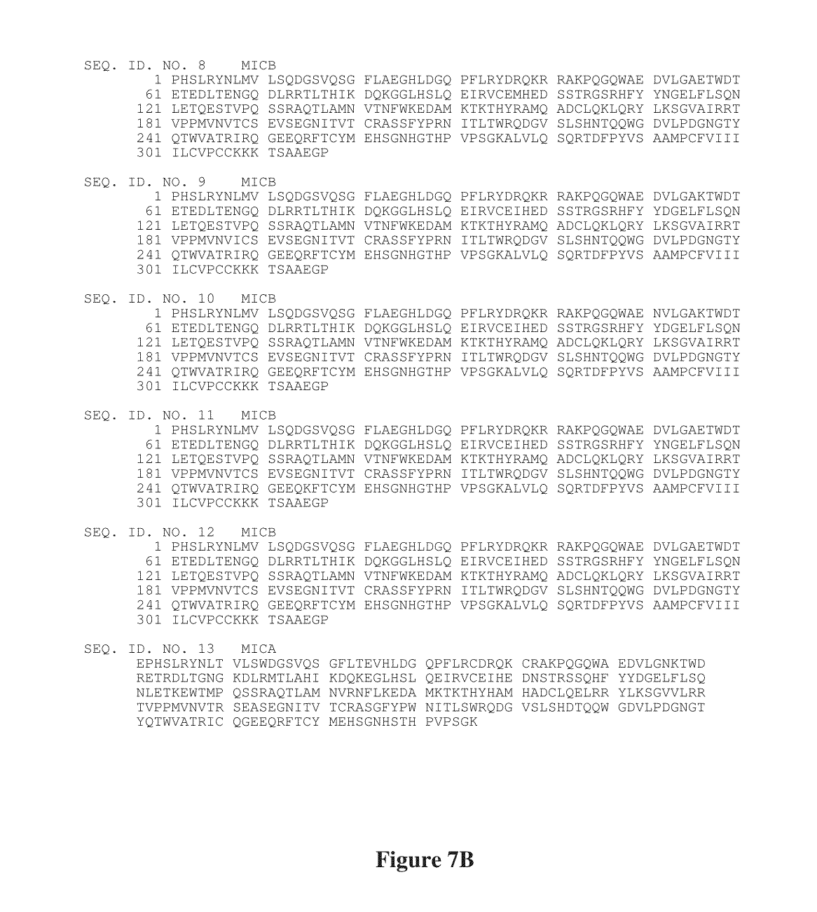

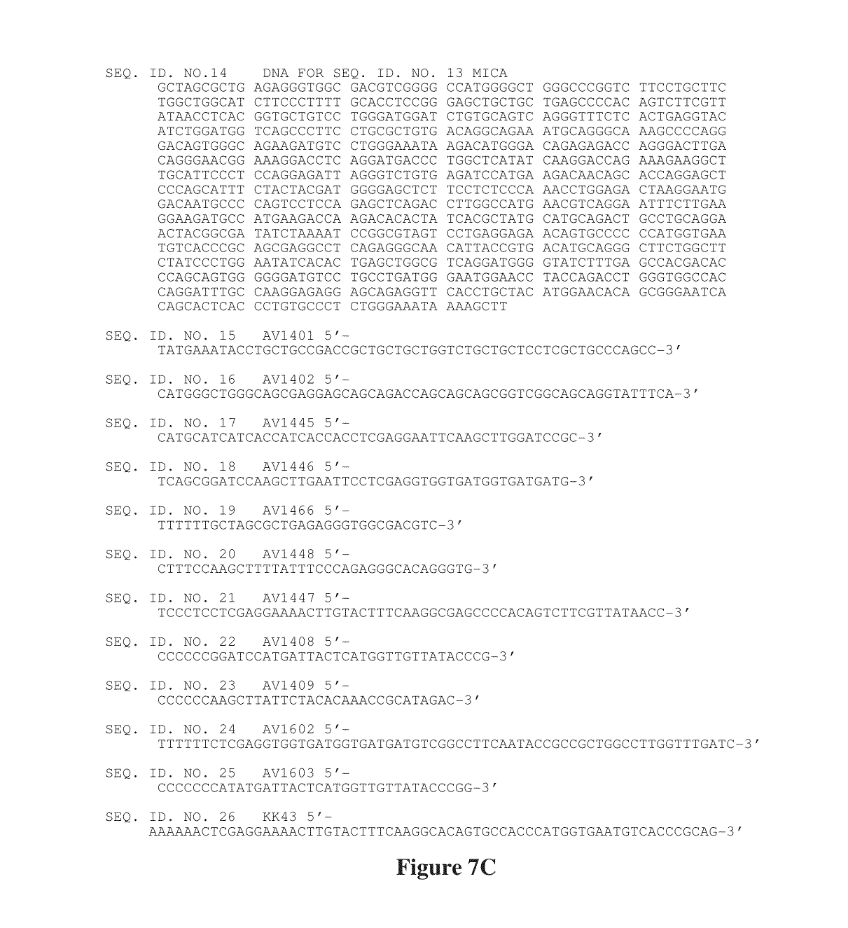

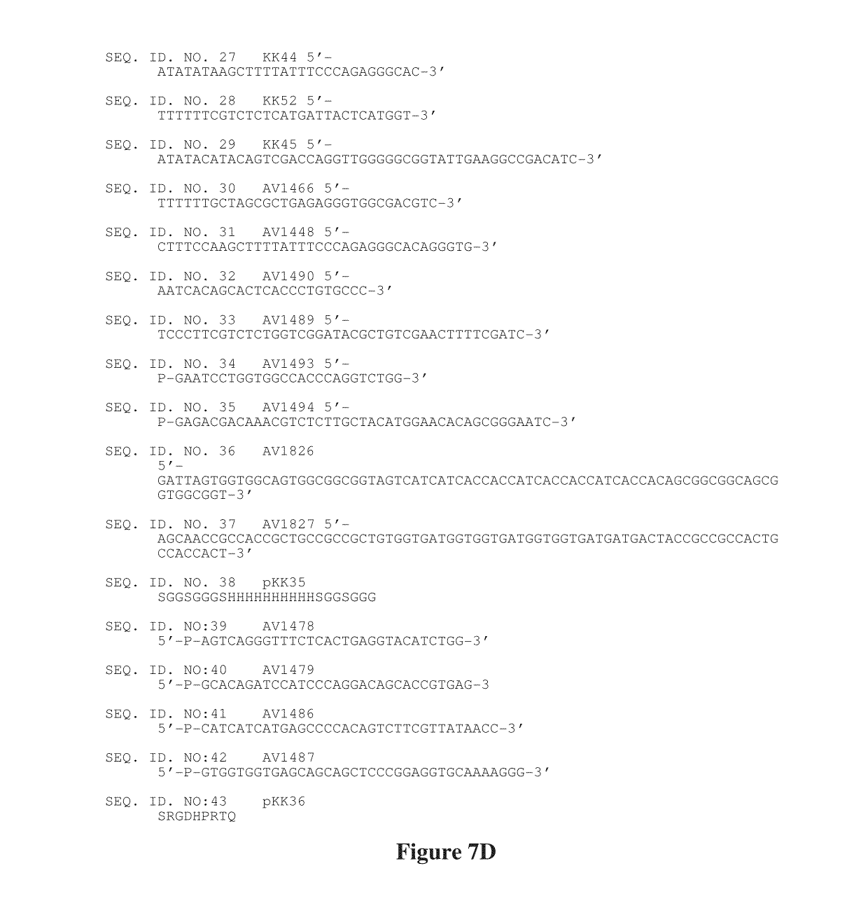

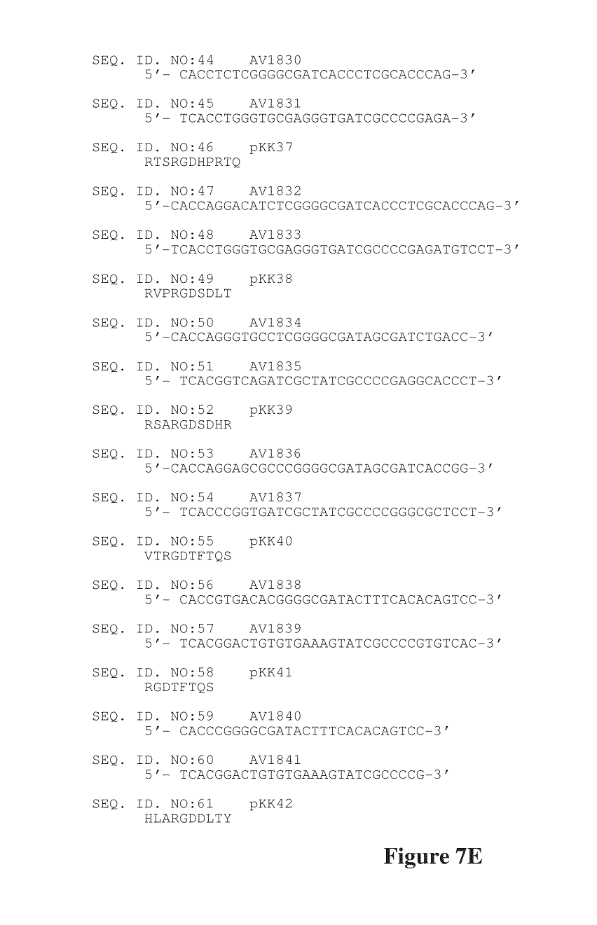

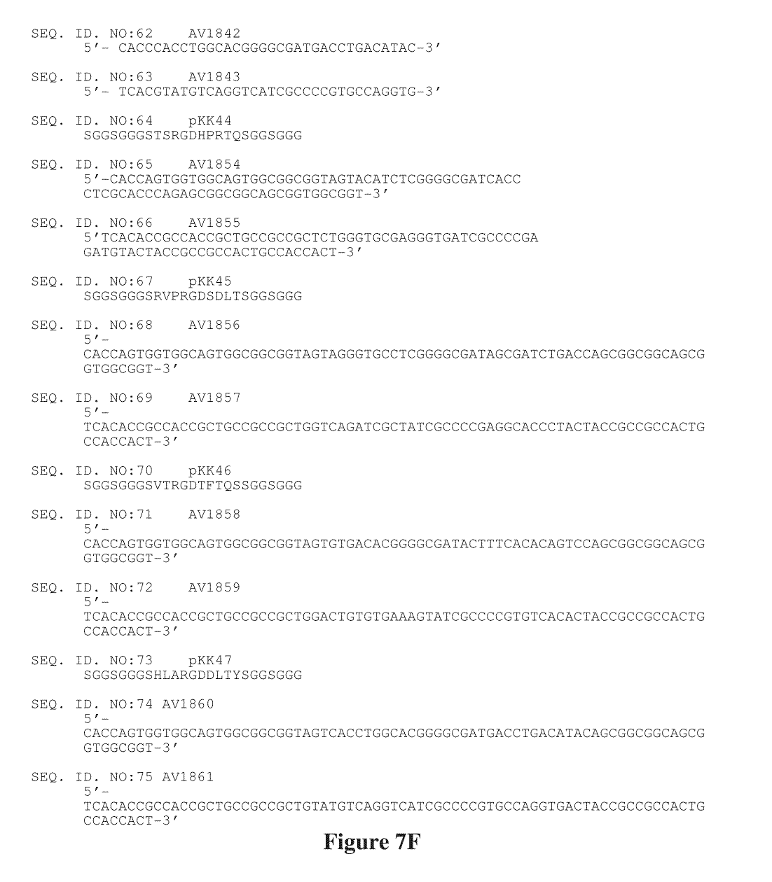

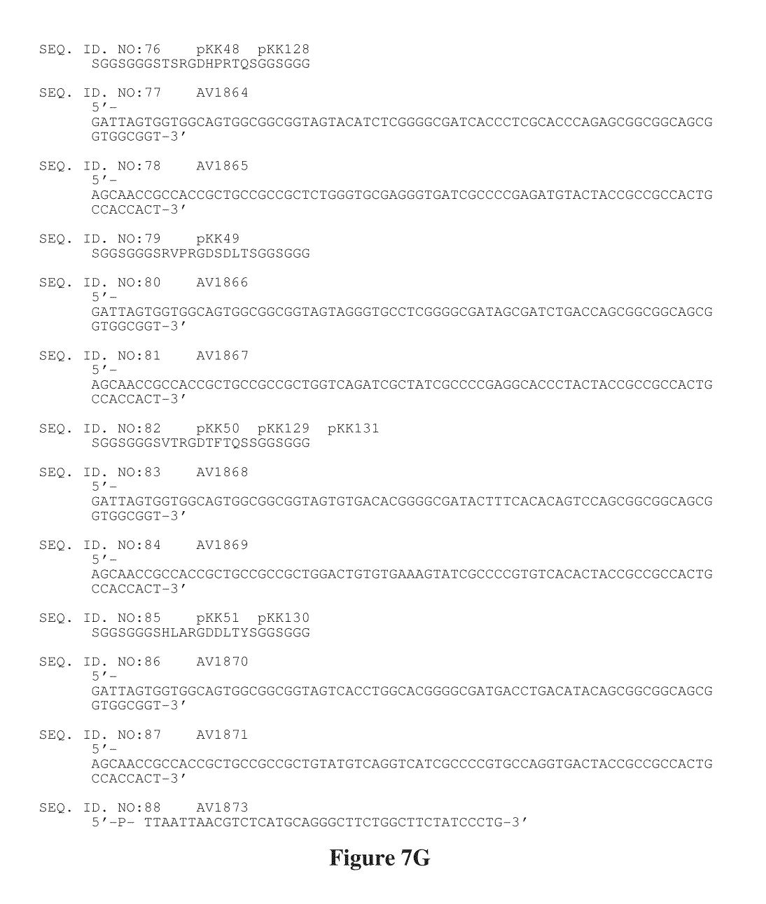

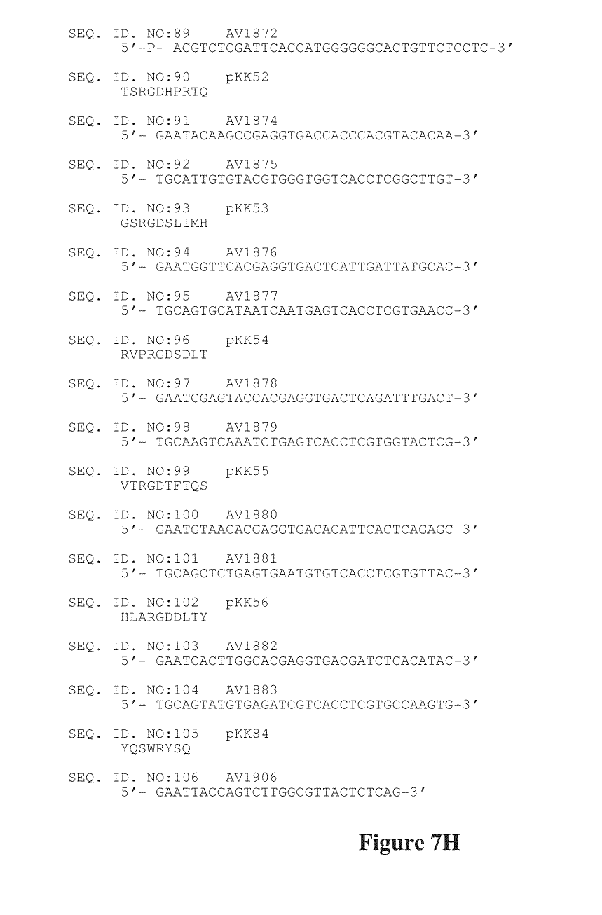

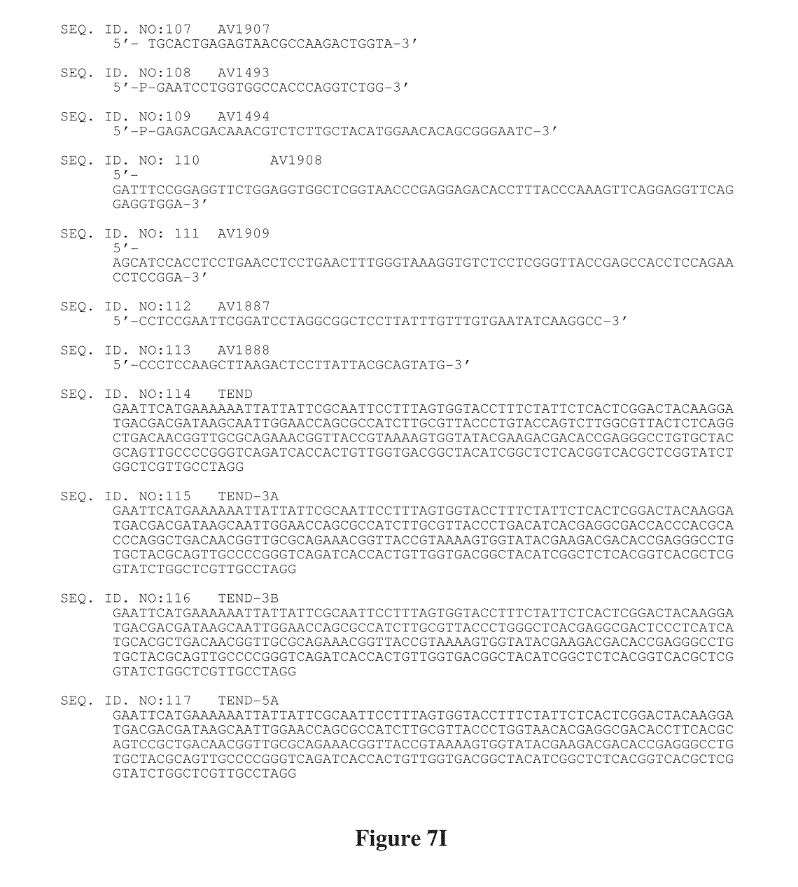

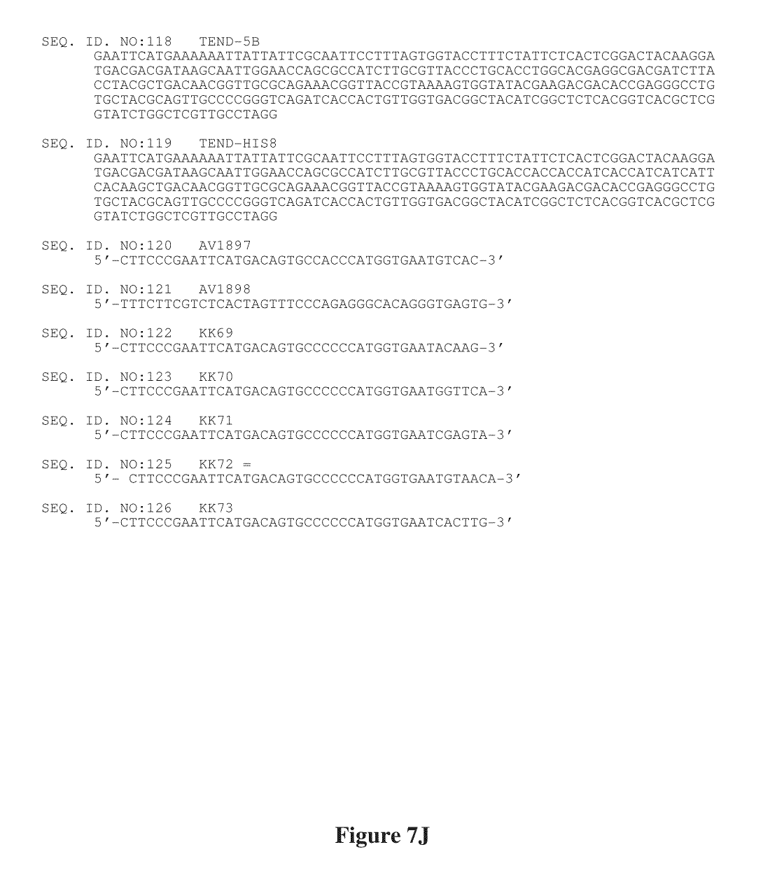

Displaying diversified proteins on the surface of bacteria, such as Escherichia coli, is an alternative approach that offers potential advantages over phage display. For example, successive rounds of optimization can be achieved without the need to make any phage or to cycle selected phage through multiple rounds of infection. And the α3 domain can be designed to be cleaved from the bacterial surface for direct biochemical or physical analyses. Although DGRs are found naturally in the genomes of over 40 bacterial species, none has been identified in E. coli. However, recently the cis and trans-acting components of a DGR from Legionella pneumophila have been shown by Miller et al to efficiently function in E. coli. Diversified α3 domains of MICA or MICB will be expressed on the surface of E. coli as fusion proteins consisting of, as a non-limiting example, the outer membrane localization and anchor domains of the EaeA intimin protein encoded by enteropathogenic E. coli (Luo Y, Frey E A, Pfuetzner R A, Creagh A L, Knoechel D G, Haynes C A, Finlay B B, Strynadka N C. (2000) Crystal structure of enteropathogenic Escherichia coli intimin-receptor complex. Nature. 405:1073-7). EaeA consists of an N-terminal segment of approximately 500 amino acids that anchors the protein to the outer membrane and is believed to form an anti-parallel β-barrel with a porin-like structure that facilitates translocation (Touze T, Hayward R D, Eswaran J, Leong J M, Koronakis V. (2004) Self-association of EPEC intimin mediated by the beta-barrel-containing anchor domain: a role in clustering of the Tir receptor. Mol Microbiol. 51:73-87). This translocation domain is followed by a series of Ig-like motifs and a C-terminal C-type lectin domain responsible for binding to the intestinal epithelial surface (FIG. 6). The elongated structure of intimin and its ability to export and anchor a heterologous protein domain to the external face of the E. coli outer membrane suggest that it is an ideal and versatile fusion partner for surface display of diversified α3 proteins (Wentzel A, Christmann A, Adams T, Kolmar H. (2001). Display of passenger proteins on the surface of Escherichia coli K-12 by the enterohemorrhagic E. coli intimin EaeA. J Bacteriol. 183:7273-84; Adams, T M, A Wentzel, and H Kolmar (2005) Intimin-Mediated Export of Passenger Proteins Requires Maintenance of a Translocation-Competent Conformation. J. of Bacteriology, 187: 522-533).

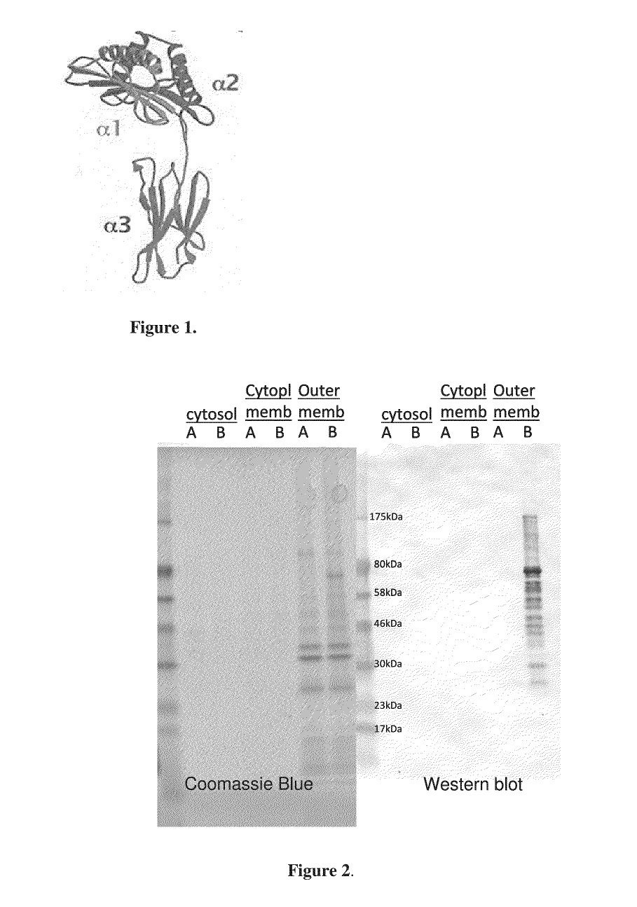

The natural orientation of MICA and MICB is such that the C-terminus is anchored to the cell membrane (type I membrane protein). The α3 domain resides between the N-terminal α1-α2 platform and the cell membrane. However, to diversify those α3 domain loops that project away from the α1-α2 platform, the opposite orientation (e.g. type II membrane protein) is desired, that is, to attach the N-terminus the linker portion of the α3 domain in FIG. 1 to EaeA so that those loops such as those located at amino acid positions 190-199, 221-228, 250-258 of SEQ ID NOs: 1-13 are readily available for binding target molecules. Such a type II membrane protein orientation is precisely that of EaeA, FIG. 6. Furthermore, the α3 domain, like EaeA, has an Ig-like motif, so that EaeA will translocate α3 domains to the E. coli surface (Li et al. 1999. Crystal structure of the MHC class I homolog MICA, a γδT cell ligand. Immunity 10: 577-584). Indeed, the ability of EaeA to translocate heterologous passenger polypeptides has been documented in the literature (Wentzel et al. 2001; Adams et al., 2005).

The EaeA-α3 fusion protein will be expressed from the araBAD promoter (P_{bad}), which responds, in a dose-dependent manner, to the concentration of arabinose added to the growth media. This will allow precise control over the density of α3 domains on the surface of bacterial cells. A diversification system, e.g. the L. pneumophila atd TR rt sequences (pDGR, FIG. 2), can be placed under control of the tightly regulated tetA promoter/operator on a multicopy plasmid. The expression of the atd TR rt sequences is induced by addition of anhydrotetracycline to the growth medium and will result in high frequency diversification of α3 VR sequences. Once diversification has been achieved, removal of inducer from the growth media will “lock” the system (α3-VR) into a stable state.

Diversification is first achieved by growing the surface display E. coli in the presence of arabinose to induce expression of the EaeA-α3 fusion protein, and anhydrotetracycline to induce diversification of α3-VR. Bacterial cells that display binding characteristics of interest can be enriched using standard methods such as Fluorescent Activated Cell Sorting (FACS) or magnetic bead separation techniques. Selected bacterial cells are amplified by growth in the presence of arabinose and the absence of anhydrotetracycline. Further enrichment steps can be included and additional rounds of optimization can be achieved by simply repeating the protocol. Importantly, α3 domains that bind to targets that are undesirable for NK or T-cell attack can be depleted from the diversified library by panning against, for example, normal tissues prior to selection for the desired binding properties. The selected α3 proteins can be cleaved from the bacterial cell surface by the addition of Factor Xa protease and then purified by affinity purification of the 6× His-tagged C-terminal domain for further characterization and use. This permits convenient biochemical and physical analyses of structure and function of the selected α3 domain. By fusing the isolated DNA encoding the desired, non-natural α3 domain to the portion of the MIC gene encoding an α1-α2 platform domain, the desired, non-natural α3 domain can then in each case be reintroduced into the rest of the soluble MIC protein via its linker or tether (amino acids 177-182) to create the desired passive NK cell vaccine with the specificity and sensitivity of the isolated α3 domain.

The selected genotype can be used to produce and isolate the non-natural or unnatural, soluble cognate MIC protein in bacteria, yeasts, insect or mammalian cells. The produced MICA can be purified to the required degree, formulated by available methods to stabilize it in vitro and in vivo, and administered parenterally or by other routes to humans or other mammals where it can diffuse to treat malignancies or viral diseases by promoting the targeted attack by the cellular components of the innate immunity system.

In some embodiments, a non-natural MIC molecule is formulated with a “pharmaceutically acceptable” excipient or carrier. Such a component is one that is suitable for use with humans or animals without undue adverse side effects. Non-limiting examples of adverse side effects include toxicity, irritation, and/or allergic response. The excipient or carrier is typically one that is commensurate with a reasonable benefit/risk ratio. In many embodiments, the carrier or excipient is suitable for topical or systemic administration. Non-limiting pharmaceutically carriers include sterile aqueous or non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyloleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Optionally, a composition comprising a non-natural MIC molecule of the disclosure may also be lyophilized or spray dried using means well known in the art. Subsequent reconstitution and use may be practiced as known in the field.

Pharmaceutical grade organic or inorganic carriers and/or diluents suitable for oral and topical use can be used to make up compositions comprising the therapeutically-active compounds. Stabilizing agents, wetting and emulsifying agents, salts for varying the osmotic pressure, or buffers for securing an adequate pH value may be included.

A non-natural MIC molecule is typically used in an amount or concentration that is “safe and effective”, which refers to a quantity that is sufficient to produce a desired therapeutic response without undue adverse side effects like those described above. A non-natural MIC molecule may be biochemically modified to alter its pharmacokinetic properties in vivo. Well-known methods to increase half-life of circulating protein molecules are to chemically attach polyethylene glycol (PEG) to the basic structure or by genetic engineering to add polymers of natural amino acids such as glycine and serine to the N-terminus, C-terminus, or internally such as in the tether between α1-α2 and α3 domains, amino acids 179-182 of SEQ ID NOS: 1-13, without affecting binding functions of the MIC protein. A non-natural MIC molecule may be used in an amount or concentration that is “therapeutically effective”, which refers to an amount effective to yield a desired therapeutic response, such as, but not limited to, an amount effective to bind target cells in order to recruit sufficient NK or T-cells to kill the target cells. The safe and effective amount or therapeutically effective amount will vary with various factors but may be readily determined by the skilled practitioner without undue experimentation. Non-limiting examples of factors include the particular condition being treated, the physical condition of the subject, the type of subject being treated, the duration of the treatment, the nature of concurrent therapy (if any), and the specific formulations employed.

### Examples

As provided herein, two technologies both well-known to those of ordinary skill in the art, were used to physically attach the genotype of a MICA α3 domain to its (binding) phenotype so as to enable selection, screening, or panning in order to isolate the DNA encoding the desired phenotype. The first was bacterial surface display, wherein the α3 domain was displayed on the surface of a bacterium harboring the DNA encoding that α3 domain. The second was bacteriophage display of the α3 domain as a chimeric phage capsid protein, wherein the encoding DNA, the genotype, was within the phage genome. The ability to use the same α3 domain genotypes to make soluble, modified human MICA molecules with the binding phenotypes reflecting those of the displayed α3 domains is also demonstrated herein.

1. Display of MIC-α3 Domain Protein on the Surface of E. coli

In brief, the DNA encoding the α3 domain was fused to a portion of the E. coli eaeA (intimin) gene, and the resulting fusion protein was expressed on the surface of E. coli in such a manner that the α3 domain was oriented with its C-terminal portion distal to the bacterial surface.

Oligonucleotides AV1401 (SEQ ID NO: 15) and AV1402 (SEQ ID NO: 16) were kinased, annealed, and ligated into an aliquot of plasmid pET30a (Novagen) which had been digested with NdeI and NcoI to create pSW249, containing a pelB secretion signal sequence.

Plasmid pSW249 was digested with NcoI and BlpI and ligated together with kinased and annealed oligonucleotides AV1445 (SEQ ID NO: 17) and AV1446 (SEQ ID NO: 18) to create pSW263. This construct contained sequence encoding six histidine residues (SEQ ID NO:127) following the pelB sequence.

A human MICA cDNA comprising a portion of 5′ untranslated sequence, signal sequence, and codons 1-276 of the mature coding sequence, followed by a stop codon, was amplified by PCR from human spleen first strand cDNA (acquired from Invitrogen) using primers AV1466 (SEQ ID NO: 19) and AV1448 (SEQ ID NO: 20).

The PCR fragment was digested with NheI and HindIII and ligated together with pCDNA5-FRT (Invitrogen) which had also been digested with NheI and HindIII to create pSW265. Three mutations the MICA coding region were corrected, G14W, A24T and E125K, by directed mutagenesis to create pSW271.

A portion of pSW271 was PCR amplified with primers AV1447 (SEQ ID NO: 21) and AV1448 (SEQ ID NO: 20). The PCR fragment consisted of a tev protease cleavage site, ENLYFQG (SEQ ID NO: 128), followed by codons 1-276 of human MICA. This PCR fragment was digested with XhoI and HindIII and ligated together with an aliquot of plasmid pSW263 which had also been digested with XhoI and HindIII to create plasmid pSW286.

The eaeA gene was amplified from E. coli EDL933 genomic DNA using AV1408 (SEQ ID NO: 22) and AV1409 (SEQ ID NO: 23) primers.

This PCR product was digested with BamHI and HindIII and ligated together with Bluescript-SK+DNA (Stratagene) which had also been digested with BamHI and HindIII to create pSW284.

A portion of plasmid pSW284 was PCR amplified using primers AV1602 (SEQ ID NO: 24) and AV1603 (SEQ ID NO: 25). The PCR fragment was digested with NdeI and XhoI and ligated together with an aliquot of pSW286 which had also been digested with NdeI and XhoI to create pSW289.

A portion of pSW289 was PCR amplified with primers kk43 (SEQ ID NO: 26) and kk44 (SEQ ID NO: 27). The resulting PCR fragment containing a tev protease cleavage site, ENLYFQG (SEQ ID NO: 128), followed by sequence encoding residues 181 through 276 of MICA (note: the codon for P183 WAS changed from CCC to CCA to break up a run of 6 C's) was digested with XhoI and HindIII and ligated together with a ˜7185 bp fragment which had been purified on an agarose gel from a digest of a separate aliquot of pSW289 digested with XhoI and HindIII to create pKK5. The 7185 bp fragment encoded EaeA 1-659 followed by GG then a factor Xa cleavage site, IEGR (SEQ ID NO: 129), then six His residues (SEQ ID NO: 127), then an XhoI site encoding LE of no function except to provide the XhoI site.

pKK5 was PCR amplified with primers kk52 (SEQ ID NO: 28) and kk45 (SEQ ID NO: 29). The PCR fragment was digested with NcoI and HindIII and ligated together with an aliquot of pBAD24 (from ATCC) which had been digested with NcoI and HindIII to create pKK29. The plasmid pKK29 was transformed according to the manufacturer's recommendations into the cloning strain, E. coli “NEB 10-beta” (catalog AV C3019H from New England BioLabs) and selected for resistance to 100 μg/ml carbenicillin.

Cytosolic proteins, inner membrane proteins, and outer membrane proteins of arabinose-induced and non-induced pKK29-transformed E. coli cells were each isolated and analyzed by SDS-PAGE. The SDS-PAGE gels were stained with Coomassie blue or western-blotted with antibody to human MICA protein.

Cells were grown in LB/Carb100 until OD₆₀₀ equaled 0.915. Aliquots of 10 mls of cells were added to each of two 50 ml conical tubes. One tube was induced with 0.002% arabinose; the other was left un-induced. Samples were incubated with shaking @ 37° C. for 1 hr.

Cells were then centrifuged for 10 min at 4000 rpm in an Eppendorf 5810R tabletop centrifuge. Supernatants were discarded and the cell pellets were gently resuspended in 6 ml FP buffer (0.1 M sodium phosphate buffer pH 7.0, 0.1 M KCl, 5 mM EDTA, 1 mM dithiothreitol, 1 mM phenylmethylsulfonyl fluoride) and transferred to 15 ml conical tubes. Cells were sonicated for 6×15 sec bursts using a Biologics Inc model 300 V/T ultrasonic homogenizer. Samples were incubated on ice between bursts. After sonication the tubes were centrifuged in the Eppendorf 5810R tabletop centrifuge at 4000 rpm for 5 minutes to remove any unbroken cells.

Supernatants were transferred to Beckman polycarbonate centrifuge tubes (catAV355631) and spun at 100,000×g for 1 hr at 4° C. in a Beckman L8-80M floor ultracentrifuge using a Type 60Ti rotor. The supernatants containing the cytosolic proteins were removed to new tubes and stored at 4° C.

The pellets of the cell membranes were resuspended in 2 mls of ME buffer (10 mM Tris-HCl pH 8.0, 35 mM MgCl₂, 1% Triton X-100). Samples shook gently for 2 hrs at 25° C. and then were re-centrifuged in the Beckman L8-80M floor ultracentrifuge using a Type 60Ti rotor at 100,000×g for 30 min at 4° C. Supernatants containing the cytoplasmic membrane Triton-soluble proteins were removed to new tubes and stored at 4° C. The final pellets containing the outer membrane proteins (Schnaitman, C A. 1971. Solubilization of the Cytoplasmic Membrane of Escherichia coli by Triton X-100. J. Bacteriology 108: 545-552) were resuspended in 0.1 ml of water and also stored at 4° C. before being subjected to analyses by SDS-PAGE and stained by Coomassie Blue or western blotted using a goat polyclonal antibody against human MICA, FIG. 2.

For SDS-PAGE analyses samples were mixed with equal volumes of Novex Tris-Glycine SDS 2× sample buffer (Invitrogen AVLC2676) and electrophoresed on 4-20% Tris-Glycine Gradient Gel (Invitrogen AVEC60285BOX). For western blotting the electrophoresed sample lanes in the slab gel were transferred to a nitrocellulose membrane (Invitrogen Nitrocellulose Membrane Filter Paper Sandwich AVLC2001) using an Invitrogen XCell II Blot Module (AVEI9051). The membrane filter was blocked overnight at 4° C. in 5% milk-Phosphate Buffered Saline, Tween-20 (PBST). Primary antibody (anti-human MICA antibody—R&D Systems AVAF1300) was used at 1:500 dilution in 5% milk-PBST. The resulting filter “blot” was incubated 2 hrs at 25° C. with gentle rocking. The filter “blot” was subsequently washed for 20 min at 25° C. with PBST after which the secondary antibody (anti-goat IgG-HRP antibody—R&D Systems AVHAF017) was added at a dilution of 1:1000 in 5% milk-PBST. The filter “blot” was rocked for 2 hrs at 25° C. and then again was washed 20 min in PBST. The filter “blot” was developed with Novex HRP Chromogenic Substrate—TMB (Invitrogen AVWP20004).

To confirm bacterial surface display of the α3 domain by an independent method, fluorescent microscopy of intact, arabinose-induced and un-induced E. coli confirmed the staining of MICA α3 on the surface of intact bacteria from the induced culture only.

2. Generation of Soluble, Non-Natural, Human MIC Proteins with Internal Targeting Domains.

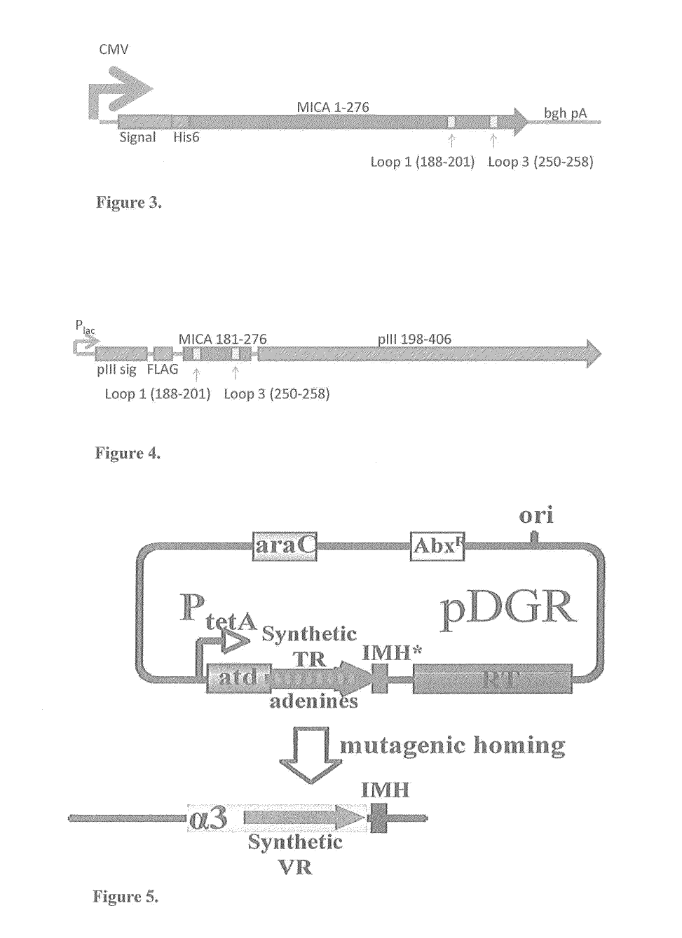

Human MICA is naturally glycosylated, although its unglycosylated form does bind its receptor, NKG2D, in vitro (Li et al., 2001). However, to be able to evaluate a human-like glycosylated form in vitro and eventually in vivo, we expressed MICA in cultured human cells. For expression, two common human alleles were inserted into the transient expression vector pcDNA5/FRT, which has a human CMV promoter and a bovine growth hormone (bgh) polyadenylation signal, FIG. 3.

### Working Plasmid Constructions

The secretion signal sequence and codons 1-276 of mature HUMMHCREP (Human MHC class I-related protein mRNA) were obtained by amplifying with a Polymerase Chain Reaction (PCR) the appropriate DNA sequence from human spleen first-strand cDNA (available from Life Technologies/Invitrogen) using primers AV1466 (SEQ ID NO: 30) and AV1448 (SEQ ID NO: 31).

The amplified DNA product was digested with NheI and HindIII restriction enzymes, and the resulting product was ligated into NheI/HindIII-digested pCDNA5/FRT (Invitrogen), to create pSW265.

The DNA of the inserted PCR product of pSW265 was sequenced and verified to include an NheI site followed by 26 bases of the 5′untranslated (UT) sequence, followed by secretion signal sequence and codons 1-276 of mature HUMMHCREP, followed by a termination codon, followed by a HindIII site. Where the coding sequence deviated from the intended sequence such that it would result in an amino acid difference if translated, the codons were changed by site-directed mutagenesis (using New England BioLabs Phusion® site-directed mutagenesis kit and appropriate primers) so that the amino acid sequence matched the relevant portion (amino acids 1-276) of the sequence described as SEQ ID NO: 13.

The corrected plasmid was designated pSW271 and contained the corrected DNA sequence encoding 26 bases of the 5′UT sequence, followed by secretion signal sequence and codons 1-276 of mature HUMMHCREP, followed by a termination codon, SEQ ID NO:14

Primers AV1490 (SEQ ID NO: 32) and AV1489 (SEQ ID NO: 33) and pSW271 were used to generate a PCR product which was subsequently digested with BamHI and BsmBI and ligated to ˜5259 bp BamHI/BsmBI fragment from pSW271. The resulting construct pSW275 lacks a BsmBI site.

Using New England BioLabs Phusion® site-directed mutagenesis kit and primers AV1493 (SEQ ID NO: 34) and AV1494 (SEQ ID NO: 35), two BsmBI sites were inserted in the MICA coding region of pSW275, creating pSW276.

Plasmid pSW267 is the same as pSW271 except MICA codon 125 is GAG (Glu) instead of AAG (Lys). It was derived from pSW265 by site-directed mutagenesis (using New England BioLabs Phusion kit with primers AV1478 (SEQ ID NO:39) and AV1479 (SEQ ID NO:40). This mutagenesis changed MICA codon 14 from GGG (Gly) to TGG (Trp) and codon 24 from GCT (Ala) to ACT (Thr).

Plasmid pSW273 was made from pSW267 by site-directed mutagenesis using primers AV1486 (SEQ ID NO:41) and AV1487 (SEQ ID NO:42). pSW273 contains six histidine codons between the signal peptide and residue 1 of the mature peptide.

Plasmid pKK33 is identical to plasmid pSW273 except a BsmBI site has been deleted from the partial hph (hygromycin resistance) gene. A PCR fragment was obtained from plasmid pSW273 by amplifying with primers AV1490 (SEQ ID NO:32) and 1489 (SEQ ID NO:33). This ˜727 bp fragment was digested with BamHI and BsmBI and was ligated together with the ˜5277 bp BamHI/BsmBI-digested fragment from pSW273. This resulted in the removal of the BsmBI site and the creation of pKK34.

The Following Describes Constructs Derived from pKK34 to Insert Binding Sequences into Loop 1 of MICA α3 Domain.

The following loop 1 constructs were generated by insertion of the indicated heterologous peptide “insert” between T189 and V200 of MICA and replacing the residues at positions 190-199 with the indicated insert.

To create pKK36, which has a sequence coding for SRGDHPRTQ (SEQ ID NO:43; referred to as loop 3.1) inserted between T189 and V200 of MICA, phosphorylated oligonucleotides AV1830 (top strand) (SEQ ID NO:44) and AV1831 (bottom strand) (SEQ ID NO:45) were ligated into BsmBI-digested pKK34.

To create pKK37, which has a sequence coding for RTSRGDHPRTQ (SEQ ID NO:46; referred to as loop 3.2) inserted between T189 and V200 of MICA, phosphorylated oligonucleotides AV1832 (top strand) (SEQ ID NO:47) and AV1833 (bottom strand) (SEQ ID NO:48) were ligated into BsmBI-digested pKK34.

To create pKK38, which has a sequence coding for RVPRGDSDLT (SEQ ID NO:49; referred to as loop 3.3) inserted between T189 and V200 of MICA, phosphorylated oligonucleotides AV1834 (top strand) (SEQ ID NO:50) and AV1835 (bottom strand) (SEQ ID NO:51) were ligated into BsmBI-digested pKK34.

To create pKK39, which has a sequence coding for RSARGDSDHR (SEQ ID NO:52; referred to as loop 3.4) inserted between T189 and V200 of MICA, phosphorylated oligonucleotides AV1836 (top strand) (SEQ ID NO:53) and AV1837 (bottom strand) (SEQ ID NO:54) were ligated into BsmBI-digested pKK34.

To create pKK40, which has a sequence coding for VTRGDTFTQS (SEQ ID NO:55; referred to as loop 5.1) inserted between T189 and V200 of MICA, phosphorylated oligonucleotides AV1838 (top strand) (SEQ ID NO:56) and AV1839 (bottom strand) (SEQ ID NO:57) were ligated into BsmBI-digested pKK34.

To create pKK41, which has a sequence coding for RGDTFTQS (SEQ ID NO:58; referred to as loop 5.2) inserted between T189 and V200 of MICA, phosphorylated oligonucleotides AV1840 (top strand) (SEQ ID NO:59) and AV1841 (bottom strand) (SEQ ID NO:60) were ligated into BsmBI-digested pKK34.

To create pKK42, which has a sequence coding for HLARGDDLTY (SEQ ID NO:61; referred to as loop 5.3) inserted between T189 and V200 of MICA, phosphorylated oligonucleotides AV1842 (top strand) (SEQ ID NO:62) and AV1843 (bottom strand) (SEQ ID NO:63) were ligated into BsmBI-digested pKK34.

To create pKK44, which has a sequence coding for SGGSGGGSTSRGDHPRTQSGGSGGG (SEQ ID NO:64; referred to as extended loop 3.2sp) inserted between T189 and V200 of MICA, phosphorylated oligonucleotides AV1854 (top strand) (SEQ ID NO:65) and AV1855 (bottom strand) (SEQ ID NO:66) were ligated into BsmBI-digested pKK34.

To create pKK45, which has a sequence coding for SGGSGGGSRVPRGDSDLTSGGSGGG (SEQ ID NO:67; referred to as extended loop 3.3sp) inserted between T189 and V200 of MICA, phosphorylated oligonucleotides AV1856 (top strand) (SEQ ID NO:68) and AV1857 (bottom strand) (SEQ ID NO:69) were ligated into BsmBI-digested pKK34.

To create pKK46, which has a sequence coding for SGGSGGGSVTRGDTFTQSSGGSGGG (SEQ ID NO:70; referred to as extended loop 5.1sp) inserted between T189 and V200 of MICA, phosphorylated oligonucleotides AV1858 (top strand) (SEQ ID NO:71) and AV1859 (bottom strand) (SEQ ID NO:72) were ligated into BsmBI-digested pKK34.

To create pKK47, which has a sequence coding for SGGSGGGSHLARGDDLTYSGGSGGG (SEQ ID NO:73; referred to as extended loop 5.3sp) inserted between T189 and V200 of MICA, phosphorylated oligonucleotides AV1860 (top strand) (SEQ ID NO:74) and AV1861 (bottom strand) (SEQ ID NO:75) were ligated into BsmBI-digested pKK34.

The Following Describes Constructs to Insert Binding Sequences into Loop 3.

The following loop 3 constructs were generated by insertion of the indicated heterologous peptide “insert” between MICA residues Isoleucine 249 and Cysteine 259 and replacing the residues at positions 250-258 with the indicated insert.

The plasmid pSW276 was digested with BsmBI and ligated to kinased and annealed oligonucleotides AV1826 (SEQ ID NO: 36) and AV1827 (SEQ ID NO: 37) to create pKK35. This plasmid contained a sequence encoding SGGSGGGSHHHHHHHHHHSGGSGGG (SEQ ID NO: 38) between MICA residues Isoleucine 249 and Cysteine 259 and replacing the residues at positions 250-258.

To create pKK48, which has a sequence coding for SGGSGGGSTSRGDHPRTQSGGSGGG (SEQ ID NO:76; referred to as extended loop 3.2sp) inserted between 1249 and C259 of MICA, phosphorylated oligonucleotides AV1864 (top strand) (SEQ ID NO:77) and AV1865 (bottom strand) (SEQ ID NO:78) were ligated into BsmBI-digested pSW276.

To create pKK49, which has a sequence coding for SGGSGGGSRVPRGDSDLTSGGSGGG (SEQ ID NO:79; referred to as extended loop 3.3sp) inserted between 1249 and C259 of MICA, phosphorylated oligonucleotides AV1866 (top strand) (SEQ ID NO:80) and AV1867 (bottom strand)(SEQ ID NO:81) were ligated into BsmBI-digested pSW276.

To create pKK50, which has a sequence coding for SGGSGGGSVTRGDTFTQSSGGSGGG (SEQ ID NO:82; referred to as extended loop 5.1sp)

inserted between 1249 and C259 of MICA, phosphorylated oligonucleotides AV1868 (top strand) SEQ ID NO:83) and AV1869 (bottom strand)(SEQ ID NO:84) were ligated into BsmBI-digested pSW276.

To create pKK51, which has a sequence coding for SGGSGGGSHLARGDDLTYSGGSGGG (SEQ ID NO:85; referred to as extended loop 5.3sp) inserted between I249 and C259 of MICA, phosphorylated oligonucleotides AV1870 (top strand) (SEQ ID NO:86) and AV1871 (bottom strand) (SEQ ID NO:87) were ligated into BsmBI-digested pSW276.

The Following Describes Constructs to Insert Binding Sequences into a “Tight” Loop 1.

The following “tight” (T) loop 1 constructs were generated by insertion of the indicated heterologous peptide “insert” between N187 and C202 of MICA, that is, residues 188-201 of MICA were replaced with the indicated insert.

The plasmid pKK34 was mutagenized with primers AV1873 (SEQ ID NO:88) and AV1872 (SEQ ID NO:89) to create pSW324, which contains convenient BsmBI sites suitable for cloning inserts between N187 and C202 of MICA.

To create pKK52, which has a sequence coding for TSRGDHPRTQ (SEQ ID NO:90; referred to as tight T-3.1) inserted between N187 and C202 of MICA, phosphorylated oligonucleotides AV1874 (top strand)(SEQ ID NO:91) and AV1875 (bottom strand) (SEQ ID NO:92) were ligated into BsmBI-digested pSW324.

To create pKK53, which has a sequence coding for GSRGDSLIMH (SEQ ID NO:93; referred to as tight T-3.5) inserted between N187 and C202 of MICA, phosphorylated oligonucleotides AV1876 (top strand)(SEQ ID NO:94) and AV1877 (bottom strand)(SEQ ID NO:95) were ligated into BsmBI-digested pSW324.

To create pKK54, which has a sequence coding for RVPRGDSDLT (SEQ ID NO:96; referred to as tight T-3.3) inserted between N187 and C202 of MICA, phosphorylated oligonucleotides AV1878 (top strand) (SEQ ID NO:97) and AV1879 (bottom strand) (SEQ ID NO:98) were ligated into BsmBI-digested pSW324.

To create pKK55, which has a sequence coding for VTRGDTFTQS (SEQ ID NO:99; referred to as tight T-5.1) inserted between N187 and C202 of MICA, phosphorylated oligonucleotides AV1880 (top strand)(SEQ ID NO:100) and AV1881 (bottom strand) (SEQ ID NO:101) were ligated into BsmBI-digested pSW324.

To create pKK56, which has a sequence coding for HLARGDDLTY (SEQ ID NO:102; referred to as tight T-5.3) inserted between N187 and C202 of MICA, phosphorylated oligonucleotides AV1882 (top strand)(SEQ ID NO:103) and AV1883 (bottom strand)(SEQ ID NO:104) were ligated into BsmBI-digested pSW324.

To create pKK84, which has a sequence coding for YQSWRYSQ (SEQ ID NO:105; loop 1 from tendamistat, referred to as tight T-AMY) inserted between N187 and C202 of MICA, phosphorylated oligonucleotides AV1906 (top strand) (SEQ ID NO:106) and AV1907 (bottom strand)(SEQ ID NO:107) were ligated into BsmBI-digested pSW324.

The Following Describes Constructs to Insert Binding Sequences into Both Loop 1 and Loop 3 of the Same Soluble MICA Molecules.

To create constructs encoding soluble MICA molecules with binding sequences inserted into both loop 1 and loop 2, we created pKK115. Vector pKK115 encodes extended 5.1sp (SEQ ID NO:82) in loop 1 and convenient BsmBI sites suitable for cloning other sequences into loop 3. Vector pKK115 was created by site-directed mutagenesis of pKK46 with primers AV1493 (SEQ ID NO:108) and AV1494 (SEQ ID NO:109).

To create pKK128, which has extended 5.1sp in loop 1 and a sequence coding for SGGSGGGSTSRGDHPRTQSGGSGGG (SEQ ID NO:76) referred to as extended loop 3.2sp) inserted between 1249 and C259 of MICA, phosphorylated oligonucleotides AV1864 (top strand) (SEQ ID NO:77) and AV1865 (bottom strand)(SEQ ID NO:78) were ligated into BsmBI-digested pKK115.

To create pKK129, which has extended 5.1sp in loop 1 and a sequence coding for SGGSGGGSVTRGDTFTQSSGGSGGG (SEQ ID NO:82; referred to as extended loop 5.1sp) inserted between 1249 and C259 of MICA, phosphorylated oligonucleotides AV1868 (top strand)(SEQ ID NO:83) and AV1869 (bottom strand)(SEQ ID NO:84) were ligated into BsmBI-digested pKK115.

To create pKK130, which has extended 5.1sp in loop 1 and a sequence coding for SGGSGGGSHLARGDDLTYSGGSGGG (SEQ ID NO:85; referred to as extended loop 5.3sp) inserted between 1249 and C259 of MICA, phosphorylated oligonucleotides AV1870 (top strand) (SEQ ID NO:86) and AV1871 (bottom strand)(SEQ ID NO:87) were ligated into BsmBI-digested pKK115.

To create pKK131, which has extended 5.1sp in loop 1 and a sequence coding for SGGSGGGSVTRGDTFTQSSGGSGGG (SEQ ID NO:82) referred to as non-homologous extended loop 5.1spNH) inserted between 1249 and C259 of MICA, phosphorylated oligonucleotides AV1908 (top strand)(SEQ ID NO: 110) and AV1909 (bottom strand)(SEQ ID NO:111) were ligated into BsmBI-digested pKK115.

The Following Describes the Cultured Human Cell Expression of the Above Created Constructs Encoding Soluble MICA Molecules with Internal Binding Inserts and the ELISA-Based Analyses of their Target Binding.

For plasmid constructs pKK35-42, pKK44-56 and pKK128-131, 90% confluent cultures of 293T cells (ATCC) in 10 cm tissue culture dishes were transfected with 10 μg of each plasmid DNA using Fugene HD transfection reagent (Roche Applied Science). After 3 days the culture medium of each culture was collected and cleared of floating cells by centrifugation at 4000 rpm in an Eppendorf 5810R tabletop centrifuge. The recovered ˜9.5 ml of each sample was concentrated using a Pierce concentrator 7 ml/9K (catalog AV89884A) spin tube. The concentrators were pre-rinsed with phosphate buffered saline (PBS). Each sample was added to the concentrator and then centrifuged for 30 min at 4000 rpm in the Eppendorf 5810R tabletop centrifuge. Each sample was washed and concentrated 3 times with 6 ml PBS—each time spinning 4000 rpm 30 min in the Eppendorf 5810R tabletop centrifuge. The concentration of soluble MICA in each resulting sample solution was estimated by an ELISA for soluble MICA. The capture agent was mouse anti-human MICA (R&D Systems part 841612), and the detection antibody was biotinylated goat anti-human MICA (R&D Systems part 841613) that was developed with Streptavidin-HRP and Ultra TMB.

The ability of the soluble MICA molecules in each of the concentrated supernatants to bind target molecules was assayed by an ELISA using the intended target proteins, integrin αVβ3 or αVβ5, as capture agents on the ELISA plate. After the respective integrins were adhered to the bottoms of the wells of the ELISA plate, the wells were washed and blocked, as well known in the field. Each sample (100 μl) of soluble MICA produced and secreted by 293T cells was added to wells containing αVβ3 or αVβ5, incubated and washed. The soluble MICA molecules captured by the integrins were detected by HRP-conjugated antibody to human MICA developed with Ultra TMB-ELISA substrate and the optical densities read. The quantity of soluble MICA in each sample was determined by the MICA-specific ELISA. The signals from the bound soluble MICA molecules (per ng of total MICA) to the specific integrins are shown. The ELISA signal from a non-binding, negative control MICA (generated by pSW273) was subtracted from each integrin binding signal. The results of the MICA products with single peptides inserts generated from pKK35-42 and pKK44-56 along with controls are shown in Table 1. The amino acid sequences and SEQ ID NOs of their specific inserts are tabulated in Table 2. Soluble MICA molecules with binding peptides inserted by genetic engineering into only one of their loops bound to the integrin targets.

TABLE 1
Integrin binding ELISA data from single inserts in soluble MICA

| plasmid | insert | insert | MICA | αvβ3 | αvβ5 | αvβ3 | αvβ5 |
|---|---|---|---|---|---|---|---|
| name | loop 1 | loop 3 | (pg/well) | signal | signal | signal/ng | signal/ng |
| pKK35 | wild type | His10sp | 170.00 | 0.419 | 0.392 | 2.46 | 2.31 |
| pKK36 | 3.1 | wild type | 29.00 | 0.550 | 0.526 | 18.97 | 18.14 |
| pKK37 | 3.2 | wild type | 34.70 | 0.777 | 0.779 | 22.39 | 22.45 |
| pKK38 | 3.3 | wild type | 9.48 | 0.291 | 0.291 | 30.70 | 30.70 |
| pKK39 | 3.4 | wild type | 26.79 | 0.482 | 0.528 | 17.99 | 19.71 |
| pKK40 | 5.1 | wild type | 24.87 | 0.485 | 0.675 | 19.50 | 27.14 |
| pKK41 | 5.2 | wild type | 29.08 | 0.516 | 0.650 | 17.74 | 22.35 |
| pKK42 | 5.3 | wild type | 24.77 | 0.526 | 0.565 | 21.24 | 22.81 |
| pKK44 | 3.2sp | wild type | 45.94 | 0.410 | 0.460 | 8.92 | 10.01 |
| pKK45 | 3.3sp | wild type | 41.32 | 0.393 | 0.410 | 9.51 | 9.92 |
| pKK46 | 5.1sp | wild type | 41.75 | 0.384 | 1.065 | 9.20 | 25.51 |
| pKK47 | 5.3sp | wild type | 29.44 | 0.294 | 0.276 | 9.99 | 9.38 |
| pKK48 | wild type | 3.2sp | 78.65 | 0.478 | 0.604 | 6.08 | 7.68 |
| pKK49 | wild type | 3.3sp | 86.75 | 0.508 | 0.478 | 5.86 | 5.51 |
| pKK50 | wild type | 5.1sp | 93.26 | 0.546 | 0.721 | 5.85 | 7.73 |
| pKK51 | wild type | 5.3sp | 96.34 | 0.694 | 1.048 | 7.20 | 10.88 |
| pKK52 | T-3.1 | wild type | 38.6l | 0.391 | 0.485 | 10.13 | 12.56 |
| pKK53 | T-3.5 | wild type | 35.16 | 0.659 | 0.800 | 18.74 | 22.75 |
| pKK54 | T-3.3 | wild type | 9.06 | 0.177 | 0.175 | 19.54 | 19.32 |
| pKK55 | T-5.1 | wild type | 33.83 | 0.475 | 0.588 | 14.04 | 17.38 |
| pKK56 | T-5.3 | wild type | 28.86 | 0.473 | 0.528 | 16.39 | 18.30 |

TABLE 2
Correlations of the plasmids expressed in 293 cells and the phage
plasmids, their trivial names, the amino acid sequences inserted
into loop 1, loop 3 or both loop 1 and loop 3, and the
corresponding SEQ ID NOs of the inserts.

| plasmids | | | | | | |
|---|---|---|---|---|---|---|
| 293 | M13 | trivial | | SEQ | | SEQ |
| cells | phage | name | Loop 1 | ID NO: | Loop 3 | ID NO: |
| SW273 | KK106 | WT | RSEASEGNIT | 13 | ICQGEEQRFT | 13 |
| | | | | (residues | | (residues |
| | | | | 190-199) | | 250-258) |
| KK35 | KK91 | 3-His10 | | | SGGSGGGSHHHHH | 38 |
| | | | | | HHHHHSGGSGGG | |
| KK36 | KK92 | 1-3.1 | SRGDHPRTQ | 43 | | |
| KK37 | KK93 | 1-3.2 | RTSRGDHPRTQ | 46 | | |
| KK38 | KK94 | 1-3.3 | RVPRGDSDLT | 49 | | |
| KK39 | KK95 | 1-3.4 | RSARGDSDHR | 52 | | |
| KK40 | KK96 | 1-5.1 | VTRGDTFTQS | 55 | | |
| KK41 | KK97 | 1-5.2 | RGDTFTQS | 58 | | |
| KK42 | KK98 | 1-5.3 | HLARGDDLTY | 61 | | |
| KK44 | KK100 | 1-3.2sp | SGGSGGGSTSRGD | 64 | | |
| | | | HPRTQSGGSGGG | | | |
| KK45 | KK101 | 1-3.3sp | SGGSGGGSRVPRG | 67 | | |
| | | | DSDLTSGGSGGG | | | |
| KK46 | KK102 | 1-5.1sp | SGGSGGGSVTRGD | 70 | | |
| | | | TFTQSSGGSGGG | | | |
| KK47 | KK103 | 1-5.3sp | SGGSGGGSHLARG | 73 | | |
| | | | DDLTYSGGSGGG | | | |
| KK48 | KK104 | 3-3.2sp | | | SGGSGGGSTSRGD | 76 |
| | | | | | HPRTQSGGSGGG | |
| KK49 | KK105 | 3-3.3sp | | | SGGSGGGSRVPRG | 79 |
| | | | | | DSDLTSGGSGGG | |
| KK50 | | 3-5.1sp | | | SGGSGGGSVTRGD | 82 |
| | | | | | TFTQSSGGSGGG | |
| KK51 | | 3-5.3sp | | | SGGSGGGSHLARG | 85 |
| | | | | | DDLTYSGGSGGG | |
| KK52 | KK107 | 1-T-3.1 | TSRGDHPRTQ | 90 | | |
| KK53 | KK108 | 1-T-3.5 | GSRGDSLIMH | 93 | | |
| KK54 | KK109 | 1-T-3.3 | RVPRGDSDLT | 96 | | |
| KK55 | KK110 | 1-T-5.1 | VTRGDTFTQS | 99 | | |
| KK56 | K111 | 1-T-5.3 | HLARGDDLTY | 102 | | |
| KK84 | KK112 | 1-T-AMY | YQSWRYSQ | 105 | | |
| KK128 | KK136 | 1-5.1sp/ | SGGSGGGSVTRGD | 70 | SGGSGGGSTSRGD | 76 |
| | | 3-3.2sp | TFTQSSGGSGGG | | HPRTQSGGSGGG | |
| KK129 | KK137 | 1-5.1sp/ | SGGSGGGSVTRGD | 70 | SGGSGGGSVTRGD | 82 |
| | | 3-5.1sp | TFTQSSGGSGGG | | TFTQSSGGSGGG | |
| KK130 | KK138 | 1-5.1sp/ | SGGSGGGSVTRGD | 70 | SGGSGGGSHLARG | 85 |
| | | 3-5.3sp | TFTQSSGGSGGG | | DDLTYSGGSGGG | |
| KK131 | KK139 | 1-5.1sp/ | SGGSGGGSVTRGD | 70 | SGGSGGGSVTRGD | 82 |
| | | 3-5.1spNH | TFTQSSGGSGGG | | TFTQSSGGSGGG | |

The results of the MICA products with binding peptides inserted into more than one loop generated from pKK128-131 along with controls are shown in Table 3. ELISA assays of the integrin target-binding of soluble MICA molecules generated from pKK128-131 were performed as follows. After the respective integrins were adhered to the bottoms of the wells of the ELISA plate, the wells were washed and blocked. Each sample (100 μl) of culture supernatant was added to wells containing αVβ3 or αVβ5, incubated and washed. The soluble MICA molecules captured by the integrins were detected by HRP-conjugated antibody to human MICA developed with Ultra TMB-ELISA substrate and the optical densities read. The quantity of soluble MICA in each sample was determined by the MICA-specific ELISA. The signal of soluble MICA molecules with more than 1 insert bound to the specific integrins (per ng of total MICA) are shown, Table 3. The amino acid sequences and SEQ ID NOs of their specific inserts are tabulated in Table 2. Those soluble MICA molecules with binding peptides inserted into more than 1 internal loop exhibited greater binding than those with only a single internal binding peptide, indicating the avidity effect of more than 1 binding motif per molecule. Furthermore, the specificity of a soluble MICA molecule for its intended target, for example αVβ5, was enhanced over the closely related target, αVβ3, when the soluble MICA molecule had more than one αVβ5-specific, internal binding peptide per MICA molecule. Such increased binding and specificity of binding were expected of a MICA molecule exhibiting avidity for its target. Thus, bivalent MICA binders were created, both duplicate binders and different binders, enabling the generation bi-specific MICA molecules and MICA molecules with avidity exceeding affinity for its target.

TABLE 3
Integrin binding ELISA data from double inserts in soluble MICA

| plasmid | insert | insert | MICA | αvβ3 | αvβ5 | αvβ3 | αvβ5 |
|---|---|---|---|---|---|---|---|
| name | loop 1 | loop 3 | (pg/well) | signal | signal | signal/ng | signal/ng |
| pSW273 | wild type | wild type | 14.7 | 0.099 | 0.100 | 6.73 | 6.80 |
| pKK46 | 5.1sp | wild type | 49.6 | 0.467 | 0.660 | 9.42 | 13.31 |
| pKK128 | 5.1sp | 3.2sp | 35.4 | 0.811 | 1.001 | 22.91 | 28.28 |
| pKK129 | 5.1sp | 5.1sp | 33.9 | 0.787 | 1.008 | 23.22 | 29.73 |
| pKK130 | 5.1sp | 5.2sp | 30.9 | 0.606 | 0.832 | 19.61 | 26.93 |
| pKK131 | 5.1sp | 5.1sp.nh | 30.7 | 0.685 | 1.071 | 22.31 | 34.89 |

### 3. Display of MICA-α3 Domain Protein on M13 Phage.

To develop a system for the isolation of new genes encoding engineered MICA molecules with desired target binding phenotypes, the DNA encoding α3 domain amino acids 181-276 was fused to capsid gene III of M13 phage (M13mp18; Smith vector type “33”) at codon position 198, FIG. 4, and generated without helper phage M13 phages with mixed wild type (wt) and α3-pIII chimeric capsids (Bass et al., 1990). Protein-stained SDS-PAGE analysis of PEG-purified phage preparations from E. coli confirmed the presence of both the fusion and wt pIII proteins in the population (data not shown).

Tendamistat is a bacterial protein with a 3-dimensional structure resembling that of the α3 domain of MICA (Pflugrath et al., 1986). Random peptides have been inserted into Tendamistat and selected by M13 phage display for binding to human integrins (McConnell and Hoess, 1995; Li et al., 2003). Tendamistat fused to pIII of M13 as positive controls for the ELISA assay. In parallel it was determined herein whether some of the same decapeptides inserted into loop 1 or into 3 of the MICA α3 domain could similarly direct M13 phage displaying the α3 domain fused to capsid pIII to bind integrins in the ELISA format.

The Following Describes the Constructions, Expression, and Assays of the M13 Phages Displaying MICA α3 Domains with Different Binding Peptides in Loop 1, Loop 3 or in Both Loop 1 and Loop 3.

The M13 phage M13KE (obtained from New England BioLabs) was used as template in a PCR amplification reaction with primers AV1887 (SEQ ID NO:112) and AV1888 (SEQ ID NO:113).

The PCR product, consisting of a C-terminal portion of M13KE gene III, was digested with EcoRI and HindIII and ligated together with EcoRI/HindIII-digested M13 phage vector M13mp18 (GenBank X02513) to create phage vector pSW326.

Phage vector pKK59 was created by inserting the synthesized sequence TEND (SEQ ID NO:114), digested with EcoRI and AvrII, into EcoRI/AvrII-digested pSW326.

Phage vector pKK60 was created by inserting the synthesized sequence TEND-3A (SEQ ID NO:115), digested with EcoRI and AvrII, into EcoRI/AvrII-digested pSW326.

Phage vector pKK61 was created by inserting the synthesized sequence TEND-3B (SEQ ID NO:116), digested with EcoRI and AvrII, into EcoRI/AvrII-digested pSW326.

Phage vector pKK63 was created by inserting the synthesized sequence TEND-5A (SEQ ID NO:117), digested with EcoRI and AvrII, into EcoRI/AvrII-digested pSW326.

Phage vector pKK64 was created by inserting the synthesized sequence TEND-5B (SEQ ID NO:118), digested with EcoRI and AvrII, into EcoRI/AvrII-digested pSW326.

Phage vector pKK65 was created by inserting the synthesized sequence TEND-His8 (SEQ ID NO:119; His8 is SEQ ID NO:130), digested with EcoRI and AvrII, into EcoRI/AvrII-digested pSW326.

To create phage vector pKK106, a ˜320 bp fragment was amplified by PCR from pKK29 using primers AV1897 (SEQ ID NO:120) and AV1898 (SEQ ID NO:121). This fragment representing the wild type MICA α3 sequence (SEQ ID NOs:1-6, 13), was then digested with EcoRI and BsmBI and ligated together with the ˜7911 bp MfeI/AvrII-digested fragment of pKK59.

To create phage vector pKK91, a ˜356 bp fragment was amplified by PCR from pKK35 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK92, a ˜305 bp fragment was amplified by PCR from pKK36 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK93, a ˜311 bp fragment was amplified by PCR from pKK37 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK94, a ˜308 bp fragment was amplified by PCR from pKK38 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK95, a ˜308 bp fragment was amplified by PCR from pKK39 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK96, a ˜308 bp fragment was amplified by PCR from pKK40 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK97, a ˜302 bp fragment was amplified by PCR from pKK41 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK98, a ˜308 bp fragment was amplified by PCR from pKK42 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK100, a ˜353 bp fragment was amplified by PCR from pKK44 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK101, a ˜353 bp fragment was amplified by PCR from pKK45 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK102, a ˜353 bp fragment was amplified by PCR from pKK46 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK103, a ˜353 bp fragment was amplified by PCR from pKK47 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK104, a ˜356 bp fragment was amplified by PCR from pKK48 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK105, a ˜356 bp fragment was amplified by PCR from pKK49 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK107, a ˜344 bp fragment was amplified by PCR from pKK52 using primers KK69 (SEQ ID NO:122) and AV1898 (SEQ ID NO:121). This fragment was then digested with EcoRI and BsmBI and ligated together with the ˜7911 bp MfeI/AvrII-digested fragment of pKK59.

To create phage vector pKK108, a ˜284 bp fragment was amplified by PCR from pKK53 using primers KK70 (SEQ ID NO:123) and AV1898 SEQ ID NO:121). This fragment was then digested with EcoRI and BsmBI and ligated together with the ˜7911 bp MfeI/AvrII-digested fragment of pKK59.

To create phage vector pKK109, a ˜284 bp fragment was amplified by PCR from pKK54 using primers KK71 (SEQ ID NO:124) and AV1898 (SEQ ID NO:121). This fragment was then digested with EcoRI and BsmBI and ligated together with the ˜7911 bp MfeI/AvrII-digested fragment of pKK59.

To create phage vector pKK110, a ˜284 bp fragment was amplified by PCR from pKK55 using primers KK72 (SEQ ID NO:125) and AV1898 (SEQ ID NO:121). This fragment was then digested with EcoRI and BsmBI and ligated together with the ˜7911 bp MfeI/AvrII-digested fragment of pKK59.

To create phage vector pKK111, a ˜284 bp fragment was amplified by PCR from pKK56 using primers KK73 (SEQ ID NO:126) and AV1898 (SEQ ID NO:121). This fragment was then digested with EcoRI and BsmBI and ligated together with the ˜7911 bp MfeI/AvrII-digested fragment of pKK59.

To create phage vector pKK112, a ˜278 bp fragment was amplified by PCR from pKK84 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK136, a ˜413 bp fragment was amplified by PCR from pKK128 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK137, a ˜413 bp fragment was amplified by PCR from pKK129 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK138, a ˜413 bp fragment was amplified by PCR from pKK130 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

To create phage vector pKK139, a ˜413 bp fragment was amplified by PCR from pKK131 using primers AV1897 (SEQ ID NO:120) and AV1898 (SEQ ID NO:121). This fragment was then digested with EcoRI and BsmBI and ligated together with the ˜7911 bp MfeI/AvrII-digested fragment of pKK59.

The phage vectors were independently transformed into NEBαF′tet competent E. coli cells. Phages produced by the transformed cells were tittered and their concentrations adjusted by dilution to 10¹³ per ml. The ability of the soluble MICA molecules in each of the phage preparations was assayed by an ELISA using the intended target proteins, integrin αVβ3 or αVβ5, as capture agents on the ELISA plate. After the respective integrins were adhered to the bottoms of the wells of the ELISA plate, the wells were washed and blocked, as well known in the field. Each sample (100 μl) of phage preparation was added to wells containing αVβ3 or αVβ5, incubated and washed. The phages captured by the integrins were detected by HRP-conjugated antibody to the M13 phage coat developed with Ultra TMB-ELISA substrate and the optical densities read. The M13 phages titers ranged from 8×10¹² to 1.1×10¹³/ml. The results of the phages displaying α3 domains with single peptides inserts generated from pKK91-98 and 100-112 are shown in Table 4. The amino acid sequences and SEQ ID NOs of their specific inserts are tabulated in Table 2. Phages displaying α3 domains with binding peptides inserted by genetic engineering into only one of their loops and fused to pIII capsid protein bind to integrin targets.

TABLE 4
Integrin binding ELISA data from single inserts in MICA α3-PIII
bacteriophage display

| phage | insert | insert | αvβ3 | αvβ5 |
|---|---|---|---|---|
| name | loop 1 | loop 3 | signal | signal |
| pKK106 | wild type | wild type | 0.000 | 0.000 |
| pKK91 | wild type | His10sp | 0.303 | 0.078 |
| pKK92 | 3.1 | wild type | 0.858 | 1.318 |
| pKK93 | 3.2 | wild type | 0.284 | 0.291 |
| pKK94 | 3.3 | wild type | 1.066 | 2.841 |
| pKK95 | 3.4 | wild type | 0.345 | 0.232 |
| pKK96 | 5.1 | wild type | 1.642 | 2.418 |
| pKK97 | 5.2 | wild type | 1.551 | 2.592 |
| pKK98 | 5.3 | wild type | 1.217 | 1.480 |
| pKK100 | 3.2sp | wild type | 0.201 | 0.186 |
| pKK101 | 3.3sp | wild type | 1.492 | 1.231 |
| pKK102 | 5.1sp | wild type | 1.016 | 0.771 |
| pKK103 | 5.3sp | wild type | 0.688 | 0.491 |
| pKK104 | wild type | 3.2sp | 0.791 | 1.121 |
| pKK105 | wild type | 3.3sp | 1.335 | 1.115 |
| pKK107 | T-3.1 | wild type | 0.999 | 1.491 |
| pKK108 | T-3.5 | wild type | −0.090 | −0.204 |
| pKK109 | T-3.3 | wild type | 0.230 | 0.167 |
| pKK110 | T-5.1 | wild type | 1.492 | 2.556 |
| pKK111 | T-5.3 | wild type | 0.707 | 0.657 |
| pKK112 | T-AMY | wild type | 0.460 | 0.515 |

The results of phages displaying α3 domains with binding peptides inserted into more than one loop generated from pKK136, 138, and 139 along with controls are shown in Table 5. The ELISA assays of the integrin target-binding of M13 phages displaying α3 domains with binding peptide inserts grafted into more than one loop were performed as follows. After the respective integrins were adhered to the bottoms of the wells of the ELISA plate, the wells were washed and blocked. Each sample (100 μl) of phage preparation was added to wells containing αVβ3 or αVβ5, incubated and washed. The phages captured by the integrins were detected by HRP-conjugated antibody to the M13 phage coat developed with Ultra TMB-ELISA substrate and the optical densities read. The M13 phages titers ranged from 8×10¹² to 1.1×10¹³/ml. The results of the phages displaying α3 domains with more than one peptide inserts generated from pKK136, 138, and 139, and controls displaying α3 domains with one insert generated from pKK93, 102, or 103 or no insert (pKK106) are shown. The amino acid sequences and SEQ ID NOs of their specific insert(s) are tabulated in Table 2. Those α3 domains with binding peptides inserted into more than 1 internal loop conveyed greater target binding of phages than do those with only a single internal binding peptide, confirming the avidity effect of more than 1 binding motif per molecule. Furthermore, the specificity of an α3 domain for its intended target, for example αVβ5, was again enhanced over the closely related target, αVβ3, when the α3 domain had more than one αVβ5-specific, internal binding peptide—the avidity effect.

TABLE 5
Integrin binding ELISA data from double inserts grafted in MICA α3-PIII
bacteriophage display

| phage | insert | insert | αvβ3 | αvβ5 |
|---|---|---|---|---|
| name | loop 1 | loop 3 | signal | signal |
| pKK106 | wild type | wild type | 0.000 | 0.000 |
| pKK93 | 3.2 | wild type | 0.616 | 0.774 |
| pKK102 | 5.1sp | wild type | 1.454 | 1.500 |
| pKK103 | 5.3sp | wild type | 0.587 | 0.814 |
| pKK136 | 5.1sp | 3.2sp | 2.462 | 4.280 |
| pKK138 | 5.1sp | 5.3sp | 0.886 | 2.748 |
| pKK139 | 5.1sp | 5.1sp.nh | 2.481 | 5.355 |

### 4. Soluble, Targeted MICA Acted as a Specific Adapter Molecule to Recruit NK Cells to Kill Target Cells.

The LIVE/DEAD® cell viability assays were carried out essentially as described by Chromy et al., 2000 and by the manufacturer's protocol (Invitrogen, Carlsbad, Calif.). Briefly, human target cells (MCF7 and HeLa) were seeded at a density of 1×10⁴ cells/well in 96-well flat-bottomed culture plates and reached 80% confluency in 2 days. The culture supernatants of 293T cells transiently transfected with pKK131 were concentrated approximately 100-fold by Pierce 9K MWCO Concentrators and the MICA concentrations determined by the ELISA specific for MICA. To demonstrate the ability of soluble, targeted MICA molecules to recruit human NK cells to kill target cells, different concentrations of the concentrated soluble MICA protein were incubated for 16 hours in different wells containing the target cells. Unbound protein was then removed by washing the wells twice with phosphate buffered saline (PBS). The target cells exposed to the soluble MICA were then treated with calcein-AM (2 μM) for 30 minutes at 37° C. to achieve green fluorescence in all living cells. Following incubation, cells were again washed twice with PBS and then exposed to live NK-92MI cells in a 10:1 and 5:1 ratio to target cells. NK-92MI cells were incubated with target cells for four hours, and then unbound NK-92MI cells were removed and target cells washed twice with PBS. Next, ethidium homodimer was added (2 μM) for 30 min at 37° C. to determine the extent of NK cell killing. Cells were washed and analyzed on a fluorescent plate reader (SoftMaxPro). Live cells and dead cells were quantified using average of red (ethidium) and green (calcein-AM) fluorescence signals from wells in the absence of NK cells and at the 5:1 and 10:1 ratios and at 0, 64 pg and 128 pg of added soluble, targeted MICA.

The red fluorescent signals from (dead) MCF cells changed from 12.6±1.1 to 12.9±2.9 to 12.0±1.0 as NK cells were added at a ratio of 0 to 5:1 to 10:1 in the absence of targeted MICA. In the presence of 64 pg of targeted MICA, red fluorescent signals from (dead) MCF cells changed from 19.9±2.4 to 27.0±1.0 to 31.0±1.6 as NK cells were added at a ratio of 0 to 5:1 to 10:1. When targeted MICA was added at 128 pg, red fluorescent signals from (dead) MCF cells changed from 25.6±2.2 to 41.0±6.7 as NK cells were added at a ratio of 5:1 to 10:1. The corresponding fluorescent (green) signals from live cells in the same wells were 5621±372, 5535±205 and 5721±335 as NK cells were added at a ratio of 0 to 5:1 to 10:1 in the absence of targeted MICA. In the presence of 64 pg of targeted MICA, green fluorescent signals from live MCF cells changed from 5028±177 to 5181±102 to 3697±591 as NK cells were added at a ratio of 0 to 5:1 to 10:1. When targeted MICA was added at 128 pg, green fluorescent signals from live MCF cells changed from 3459±394 to 2191±331 as NK cells were added at a ratio of 5:1 to 10:1.

The fluorescent signals from HeLa cells, which do not express the targeted integrin, did not indicate increased killing by NK-92MI cells as increasing quantities of targeted MICA were added to the wells.

### 5. The Modified α1-α2 Domain of MIC Protein.

The α1-α2 domain of MIC proteins is a natural ligand for the NKG2D receptor and possesses an affinity for NKG2D sufficient for physiologic activation of NK cells and stimulating lysis of cells expressing native full-length MIC proteins irreversibly tethered to the two-dimensional plasma membrane surface of a “target cell” (Bauer S, Groh V, Wu J, Steinle A, Phillips J H, Lanier L L, Spies T. Science. 1999 Jul. 30; 285(5428):727-9). However, because engineered soluble MIC proteins of the instant invention reversibly bind specific target antigens on the surface of a target cell, the binding affinity of the engineered soluble MIC protein to NKG2D will directly affect the stability of the soluble MIC-dependent complex formed between NK cells and cells expressing target antigens. Especially if the affinity between sMICA and NKG2D is increased by a substantially slower dissociation rate or off-rate of the modified sMICA from NKG2D, the NK cell-based killing would be expected to be greater at lower densities of soluble MIC molecules bound to a target cell. Prior to the instant invention there had not been identified any α1-α2 mutations that alter the killing activity of soluble MIC proteins or significantly reduce the binding off-rate to enhance affinity of MIC proteins to NKG2D. A computational design effort showed that three mutations in the α1-α2 domain of wild-type MICA: N69W, K152E, and K154D (WED-MICA) in combination can moderately affect NKG2D binding affinity by affecting the stability of unbound MICA and thereby its association rate or on-rate of binding to NKG2D (Lengyel C S, Willis L J, Mann P, Baker D, Kortemme T, Strong R K, McFarland B J. J Biol Chem. 2007 Oct. 19; 282(42):30658-66. Epub 2007 Aug. 8); Subsequent extensive computational design work by the same group scanning by iterative calculations 22 amino acid positions of MICA theoretically in contact with NKG2D, according to the published structural descriptions (Li P, Morris D L, Willcox B E, Steinle A, Spies T, Strong R K. Nat Immunol. 2001 May; 2(5):443-451), showed experimentally that when combined with the earlier designed 3 changes, further rational, iterative computational design of MICA qualitatively changed its affinity for NKG2D from weak (Kd ˜2.5 μM) to moderately tight (Kd=51 nM) with a total of seven combined mutations (Henager, Samuel H., Melissa A. Hale, Nicholas J. Maurice, Erin C. Dunnington, Carter J. Swanson, Megan J. Peterson, Joseph J. Ban, David J. Culpepper, Luke D. Davies, Lisa K. Sanders, and Benjamin J. McFarland, 2102, Combining different design strategies for rational affinity maturation of the MICA-NKG2D interface. Protein Science 21:1396-1402). In contrast, the experimental approach described in this example selected amino acid modifications of MICA that slowed the off-rate between the α1-α2 domain of MICA and NKG2D, commencing with a MICA stabilized by the 3 WED changes (Lengyel C S, Willis L J, Mann P, Baker D, Kortemme T, Strong R K, McFarland B J. J Biol Chem. 2007 Oct. 19; 282(42):30658-66. Epub 2007 Aug. 8).

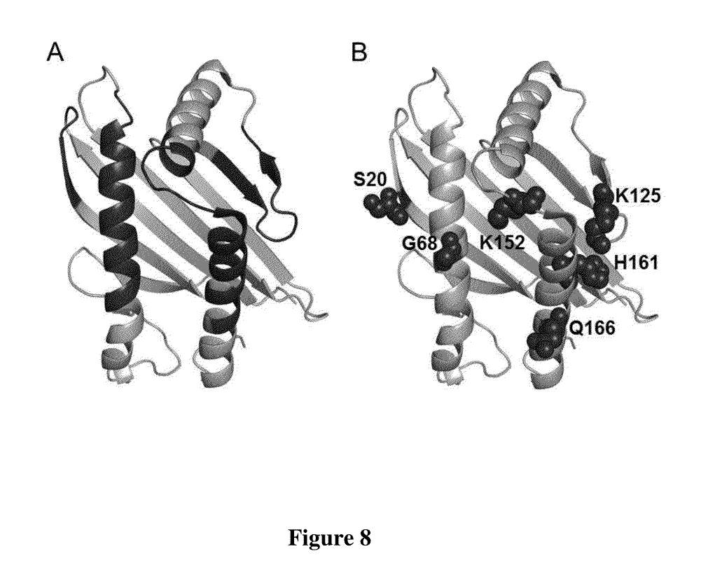

This example relates to modifying the NKG2D binding affinity of soluble MIC proteins through engineering specific mutations at selected amino acid positions within the α1-α2 domain that influence the off-rate binding kinetics and thereby alter the NK cell-mediated killing activity of the invented non-natural, targeted MIC molecules. To engineer soluble non-natural α1-α2 domains with altered affinity to NKG2D 57 residues in the α1-α2 domain were chosen for extensive mutagenesis (FIG. 8). Synthetic DNA libraries coding for the α1-α2 domain and containing NNK mutagenic codons at each of the 57 amino acid positions were synthesized, individually cloned as fusions to the pIII minor coat protein of M13 phage, and phage particles displaying the mutagenized α1-α2 variants were produced in SS320 E. coli cells according to standard methodologies (Andris-Widhopf, J., Steinberger, P., Fuller, R., Rader, C., and Barbas, C. F., 3rd. (2011) Generation of human Fab antibody libraries: PCR amplification and assembly of light- and heavy-chain coding sequences, Cold Spring Harbor protocols 2011). The α1-α2 phage libraries were sorted for increased binding affinity using recombinant biotinylated NKG2D as the target antigen and cycled through iterative rounds of intentionally prolonged binding, prolonged washing, and eluting of the phage clones in order to select high affinity variants enriched for slow dissociation- or off-rates. A set of specific amino acid mutations occurred at high frequencies at 6 positions in α1-α2 and were selected as preferred amino acid substitutions with enhanced NKG2D binding affinity (FIG. 8, Table 6).

TABLE 6
Selected affinity mutations at the indicated 6 amino acid positions of
the α1-α2 domain of MIC. The amino acids of SEQ ID NO: 135
at each of the 6 positions are shown in bold in the first row of the table.
The identified affinity mutations are listed in decreasing frequency
from top to bottom. All amino acids are represented by the single
letter IUPAC abbreviations.

| S20 | G68 | K125 | E152 | H161 | Q166 |
|---|---|---|---|---|---|
| P | L | L | T | R | F |
| T | F | R | V | S | S |
| D | S | F | G | A | H |
| A | A | T | F | K | Y |
| L | Y | A | Y | G | W |
| N | I | N | A | L | V |
| | E | V | Q | F | L |
| | T | Y | D | Y | M |
| | W | I | I | | |
| | | S | N | | |
| | | | S | | |
| | | | H | | |
| | | | M | | |
| | | | P | | |

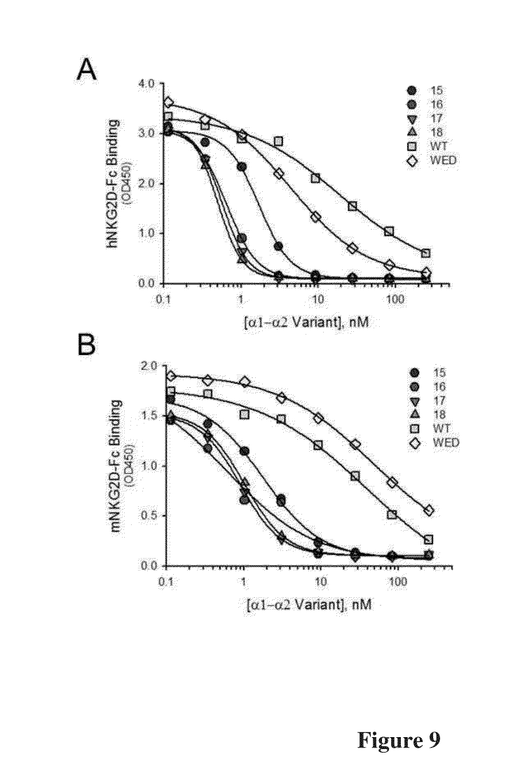

We synthesized DNA polynucleotides (SEQ ID NOs. 127-130) encoding the α1-α2 domains of 4 representative variants 15, 16, 17, 18 that contained different combinations of specific discovered mutations (Table 7). As for the NKG2D ligands (NKG2DLs) in the above example, we attached polypeptides directly to each of these 4 modified α1-α2 NKG2DLs using a linker peptide. The 4 His-tagged proteins (SEQ ID NOs.: 131-134) were expressed in insect cells and purified to characterize their NKG2D binding affinities and kinetic binding parameters. Using a competitive binding ELISA, we determined the relative NKG2D binding affinities of the 4 modified α1-α2 variants. A soluble wild type (WT) NKG2DL, sMICA protein (SEQ ID NO.:13), was coated in all wells of a maxisorp ELISA plate to provide a binding partner for the human NKG2D-Fc reagent. Solutions of the four α1-α2 variants as well as WT and WED—α1-α2 domains (SEQ ID NO.: 135) were titrated in the ELISA wells and allowed to competitively inhibit 2 nM human NKG2D-Fc binding to the WT sMICA coated on the plate. The level of human NKG2D-Fc that bound to the WT NKG2DL on the plate was detected using an anti-Fc-HRP antibody. FIG. 9A shows variants 16, 17, and 18 exhibited IC₅₀ values of 0.7, 0.6, 0.5 nM while variant 15 exhibited an IC₅₀ value of 1.7 nM, all possessing significantly better binding to NKG2D, 27, 32-, 38- and 11-fold better, than WT NKG2DL, respectively, as well as substantially better than WED-MICA (Table 8). Importantly, the relative IC₅₀ differences also translated to better binding to murine NKG2D-Fc (FIG. 9B), and demonstrated the ability to improve binding of soluble, modified α1-α2 domains across human and non-human NKG2D receptors, an important property for preclinical drug development.

TABLE 7
Sequences of specific α1-α2 domain variants. The specific amino acid
substitutions for variants 15, 16, 17, and 18 are listed relative to
the amino acids of SEQ ID NO.: 135 in bold. All amino acids are
represented by the single letter IUPAC abbreviations.

| Variant | SEQ ID NO.: | S20 | G68 | K125 | H161 |
|---|---|---|---|---|---|
| 15 | 131 | S | G | N | R |
| 16 | 132 | S | G | L | R |
| 17 | 133 | S | L | L | R |
| 18 | 134 | P | L | L | R |

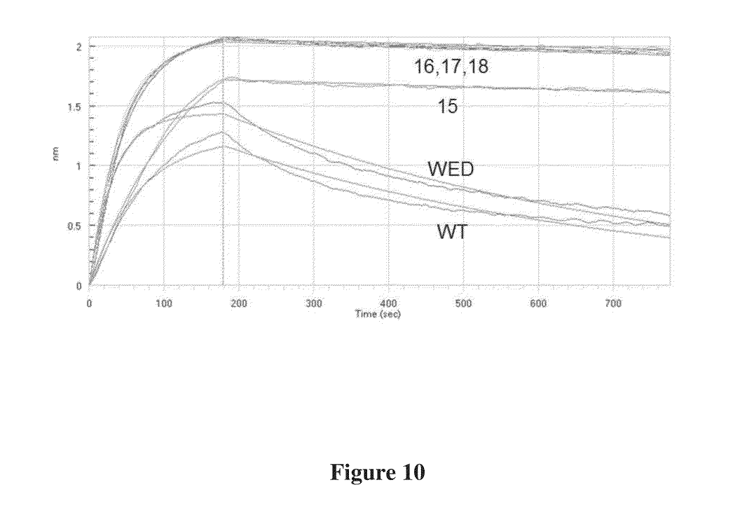

TABLE 8
Equilibrium and kinetic binding parameters for α1-α2 variants.
IC₅₀ values were derived from 4-parameter fits to the
competition binding titrations (FIG. 9A) and the kinetic binding
parameters were derived from single exponential fits to the binding
kinetics (FIG. 10). Equilibrium binding constants (K_{d}) were derived
from the kinetic binding parameters using the equation K_{d} = k_{OFF}/k_{ON}.

| | | Kinetic Binding Parameters | | |
|---|---|---|---|---|
| α1-α2 Variant | IC₅₀ (nM) | k_{ON} (M⁻¹s⁻¹) | k_{OFF} (s₋₁) | K_{d} (nM) |
| WT | 19.4 | 1.3 × 10⁵ | 1.8 × 10⁻³ | 13.8 |
| WED | 4.4 | 2.9 × 10⁵ | 1.7 × 10⁻³ | 5.9 |
| 15 | 1.7 | 0.7 × 10⁵ | 1.1 × 10⁻⁴ | 1.5 |
| 16 | 0.7 | 2.0 × 10⁵ | 0.9 × 10⁻⁴ | 0.5 |
| 17 | 0.6 | 2.0 × 10⁵ | 0.7 × 10⁻⁴ | 0.4 |
| 18 | 0.5 | 2.3 × 10⁵ | 0.9 × 10⁻⁴ | 0.4 |

In order to understand the kinetic basis for the altered affinities, both the on-rates and off-rates for the α1-α2 variant NKG2DLs binding to surface coated biotinylated human NKG2D were measured using biolayer interferometry (Octet) at 100 nM of each of the modified α1-α2 proteins. Consistent with results from the IC₅₀ ELISAs, variants 16, 17 and 18 each displayed significant reductions in the off-rate (18-fold relative to WT), which is largely responsible for the affinity increase (˜30-fold relative to WT α1-α2)(FIG. 10; Table 8). Although variant 15 displayed a similar slow off-rate as did 16, 17, and 18, its on-rate was decreased, resulting in an affinity stronger than WT but weaker variants 16, 17 and 18. Because the only difference between variant 15 (SEQ ID NO.:131) and 16 (SEQ ID NO.:132) was K125N versus K125L, the mutation at position 125 clearly altered the on-rate while the decreased off-rate was attributed to the H161R mutation. Therefore, while the selected set of MIC protein mutations (Table 6) was used to increase the α1-α2 affinity for NKG2D through significant off-rate reduction, certain substitutions also altered the on-rate resulting in a range of incremental affinity increases that we showed in this invention to have differential activity in the NK cell-mediated killing assays as described below.

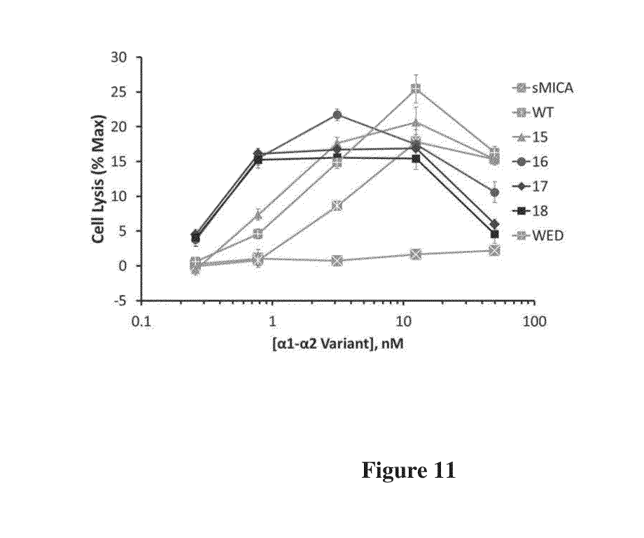

The ability of the α1-α2 affinity variants to redirect NK cell-mediated lysis of FGFR3-expressing target cells was demonstrated in vitro in a calcein-release assay. The human Natural Killer (NK) cell line, NKL, was co-cultured with calcein-loaded P815 target cells ectopically expressing FGFR3 and titrated with soluble modified MIC proteins. The results in FIG. 11 showed that the killing activities of the FGFR3-specific soluble MIC variants correlated with their engineered α1-α2 affinities. Specifically, variants 16, 17, and 18 exhibited ˜15-fold more killing than WT at 0.78 nM. The WED-MICA (SEQ ID NO.:135) was only slightly better than WT. Therefore, the invention describes amino acid substitutions within the α1-α2 domain that increased the NKG2D binding affinity by reducing the off-rate of soluble MIC protein binding to human NKG2D and consequentially led to the predictably increased killing potency. WED-MICA, which exhibited somewhat greater affinity than WT MICA to human NKG2D (FIG. 9A) by increasing on-rate rather than reducing off-rate (FIG. 10), did not exhibit substantial improvement of target cell killing (FIG. 11). Furthermore, as shown in FIG. 9B, WED-MICA exhibited substantially poorer binding to murine NKG2D than even WT MICA, while variants 15, 16, 17, and 18 each exhibited greater affinity for both human and murine NKG2D, FIG. 9A-B.

These α1-α2 NKG2DL affinity variants 15, 16, 17, and 18 enhanced the binding affinity of the attached polypeptide to the NKG2D receptor and thereby enhanced NK cell-mediated lysis of targeted cells, FIG. 11.

6. A Soluble MICA Protein Containing a Modified Antibody Variable Fragment (iFv) as a Target-Binding Peptide Inserted into a Solvent-Exposed Loop of the α3 Domain

Useful polypeptides that possess antigen binding function can be derived from the variable domains of antibodies. The antibody light and heavy chain Ig variable domains contain complementarity determining regions (CDRs) that impart antigen binding specificity and affinity. These two antibody variable domains each with 3 CDRs can be fused in tandem, in either order, using a single linker segment consisting of 10-25 amino acids rich in glycines and serines to create a single-chain variable fragment (scFv) polypeptide comprising both heavy and light chain variable domains (Bird, R. E., Hardman, K. D., Jacobson, J. W., Johnson, S., Kaufman, B. M., Lee, S. M., Lee, T., Pope, S. H., Riordan, G. S., and Whitlow, M. (1988) Single-chain antigen-binding proteins, Science 242, 423-426; Huston, J. S., Levinson, D, Mudgett-Hunter, M, Tai, M-S, Novotny, J, Margolies, M. N., Ridge, R., Bruccoleri, R E., Haber, E., Crea, R., and Opperman, H. (1988). Protein engineering of antibody binding sites: Recovery of specific activity in an anti-digoxin single-chain Fv analogue produced in Escherichia coli. PNAS 85: 5879-5883) This format enables one ordinarily skilled in the art of recombinant DNA technology to genetically fuse a scFv to the N- or C-terminus of a parent protein in order to impart to the parent protein the antigen binding properties of the scFv. However, a traditional scFv is not capable of being inserted into a loop region embedded within a protein fold of the parent without disrupting or destabilizing its fold(s) and the inserted framework properly positioning the CDRs and perhaps the CDRs per se.

To insert the variable fragment of an antibody containing up to 6 CDRs into one or more loop regions of the MICA α3 domain without disrupting folds of the variable fragment or MICA α3 domain we invented a new class of antigen-binding peptides, derived from the light and heavy chain antibody variable domains. The new structures contained two linkers, each similar to those linkers used in the art to a construct scFv, rather than the traditional single linker of scFv structures, and a split variable domain. Conceptually the canonical termini of the variable light (VL) and heavy (VH) domains were fused into a continuous or “circular” peptide. That circular peptide structure containing all 6 CDRs of the Fv could then be conceptually split at one of several possible novel sites to create an insertable Fv (iFv). The non-natural split site could be created within either the light or the heavy chain variable domain at or near the apex or turn of a loop to create new, unique N- and C-termini proximally positioned to each other so as to be insertable into a loop of the MICA α3 domain. This new class of peptides is called an insertable variable fragment (iFv).

As specific examples, we synthesized a 1126 bp and a 1144 bp DNA fragment (SEQ ID NO:136 and 137, respectively) encoding in the following order: the α3 domain amino acid 182 to amino acid 194 (the beginning of loop 1), no spacer or a GGS amino acid spacer region (SR), an iFv peptide based on the structure of a Fibroblast Growth Factor Receptor 3 (FGFR3)-binding antibody (MAbR3), no spacer or another GGS spacer region, the distal portion of loop 1 of the α3 domain starting at amino acid 196 and including the remaining carboxy-terminal portion of the α3 domain to amino acid 276 of a soluble MICA molecule (Qing, J., Du, X., Chen, Y., Chan, P., Li, H., Wu, P., Marsters, S., Stawicki, S., Tien, J., Totpal, K., Ross, S., Stinson, S., Doman, D., French, D., Wang, Q. R., Stephan, J. P., Wu, Y., Wiesmann, C., and Ashkenazi, A. (2009) Antibody-based targeting of FGFR3 in bladder carcinoma and t(4; 14)-positive multiple myeloma in mice, The Journal of clinical investigation 119, 1216-1229). Each synthetic, double stranded DNA polynucleotide then encoded a polypeptide that contained 6 CDRs in the form of an iFv inserted into loop 1 of the MICA α3 domain (FIG. 1).

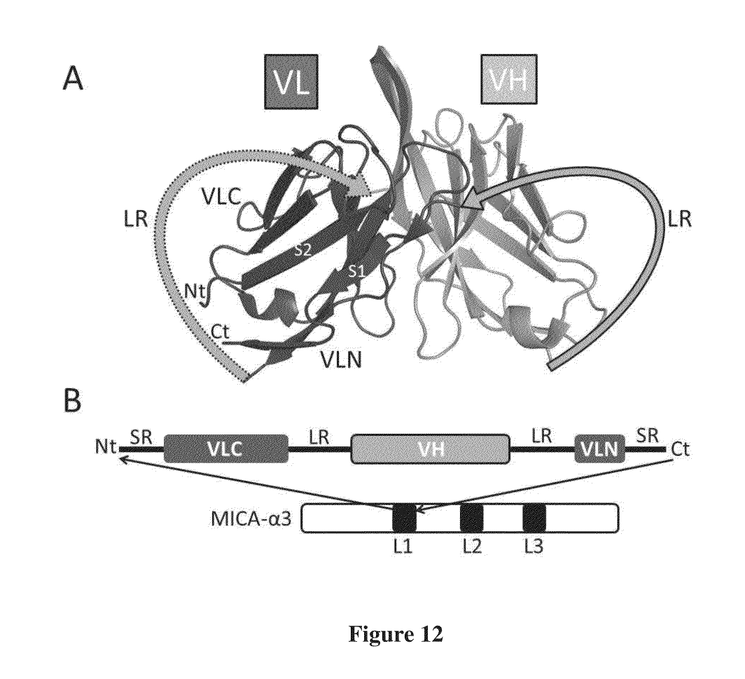

This iFv peptide itself (SEQ ID NO.:138), encoded by SEQ ID NO.:139, contained two identical, typical linker regions (LR) corresponding to residues GGSSRSSSSGGGGSGGGG (SEQ ID NO.:140). One LR joined the C-terminus of VL to the N-terminus of the VH domain, and the second LR joined the C-terminus of the VH domain to the N-terminus of VL. Conceptually this new structure is the continuous or “circular” peptide referred to above and contains 6 CDRs of the starting Fv. The variable VL chain of the antibody was effectively split within the loop region between beta-strands 1 and 2 (S1 and S2) and thereby created a new N-terminal segment (VLN) and a new C-terminal segment (VLC) with an accompanying pair of new, non-natural C- and N-termini, respectively, FIG. 12A. This pair of termini created a sole site for attachment or conjugation of the iFv to the recipient molecule such as a protein. The schematic of the inserted iFv in the parent α3 domain is shown in FIG. 12B.

To produce the soluble MICA proteins with a heterologous iFv peptide inserted into the α3 domain we generated a baculoviral expression vector to accommodate the DNA fragments (SEQ ID NO.s:136 and 137) encoding the α3-iFv.1 (SEQ ID NO.:141) and α3-iFv.2 (SEQ ID NO.:142), respectively. The DNA fragments were amplified by PCR, digested using NcoI and EcoRI restriction enzymes, and subcloned into the baculoviral expression vector, SW403, replacing the wild-type α3 domain. SW403 is a baculoviral expression vector derived from pVL1393 (Invitrogen, Inc.) into which wild-type sMICA (residues 1-276) had previously been cloned using 5′ BamHI and 3′ EcoRI sites. The new expression vector was co-transfected with baculoviral DNA into SF9 insect cells, baculovirus was grown for two amplification cycles and used to express the His-tagged MICA-α3-iFv proteins in T.ni insect cells according to manufacturer's protocol (Invitrogen). The expression was carried out in a 100 mL volume for three days and the growth medium was harvested for purification of the secreted soluble protein using Ni-affinity chromatography. Monomeric MICA-α3-iFv was purified to >90% purity with the expected molecular weight of 60.9 kDa as determined by SDS-PAGE. Functional characterization was carried out using binding ELISAs and in vitro target cell killing assays.

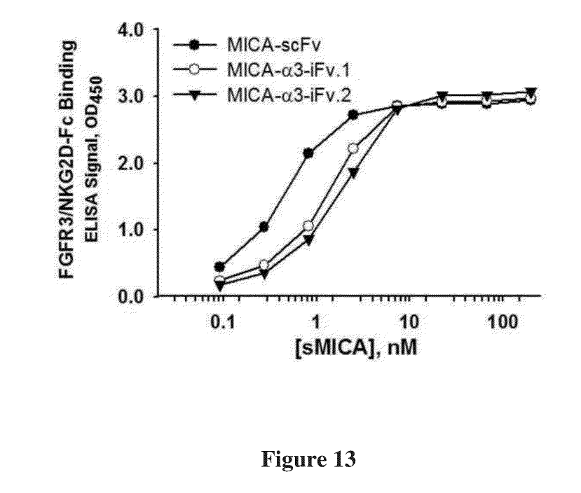

The purified MICA-α3-iFv proteins were tested in a FGFR3-binding ELISA to confirm simultaneous binding to the FGFR3 target and the NKG2D receptor. FGFR3 in phosphate buffered saline (PBS) was coated onto Maxisorp plates at 2 ug/ml concentration. Each MICA protein was titrated, allowed to bind FGFR3 for 1 hour, and washed to remove unbound sMICA protein. The bound MICA-α3-iFv protein was detected using NKG2D-Fc and anti-Fc-HRP conjugate. FIG. 13 shows that the binding of both MICA-α3-iFv.1 and MICA-α3-iFv.2 to FGFR3 was comparable to that of a MICA-scFv, made by fusing to the C-terminus of soluble MICA a traditional scFv constructed from MAbR3. These ELISA results also indicated that both the FGFR3 and NKG2D binding specificities were retained by the modified MICA and demonstrated that the inserted iFv peptide was functional using different spacer formats.

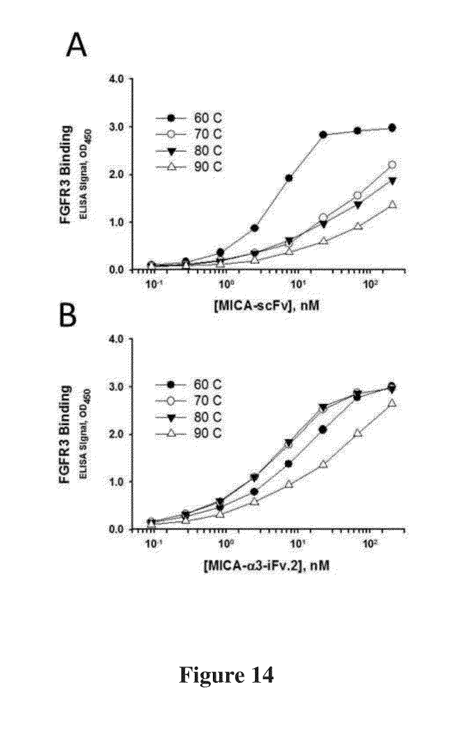

We tested and compared the thermal stability of MICA-α3-iFv.2 to that of MICA-scFv. Both proteins were subjected for 1 hr to increasing temperatures from 60-90° C. and then allowed to equilibrate to room temperature for 1 hour before being assayed for binding properties by ELISA. The results in FIG. 14 showed that MICA-α3-iFv.2 can be subjected to temperatures as high as 80° C. with no loss in specific binding to FGFR3. The traditional MICA-scFv lost binding activity at 70° C. This result indicated that soluble MICA containing the invented iFv format is significantly more stable than terminal fusions of a traditional scFv (Miller, B. R., Demarest, S. J., Lugovskoy, A., Huang, F., Wu, X., Snyder, W. B., Croner, L. J., Wang, N., Amatucci, A., Michaelson, J. S., and Glaser, S. M. (2010) Stability engineering of scFvs for the development of bispecific and multivalent antibodies, Protein engineering, design & selection: PEDS 23, 549-557; Weatherill, E. E., Cain, K. L., Heywood, S. P., Compson, J. E., Heads, J. T., Adams, R., and Humphreys, D. P. (2012) Towards a universal disulphide stabilised single chain Fv format: importance of interchain disulphide bond location and vL-vH orientation, Protein engineering, design & selection: PEDS 25, 321-329.

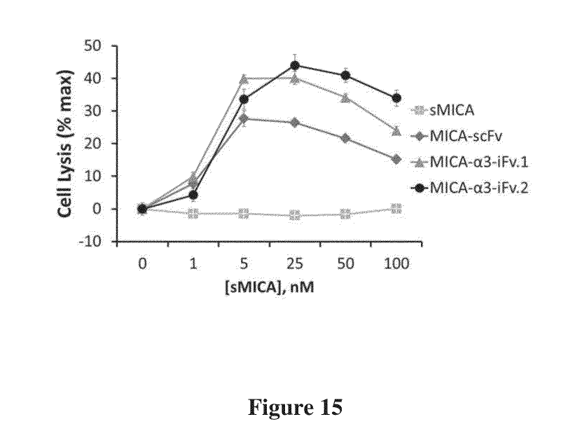

The ability of MICA-α3-iFv to redirect NK cell-mediated lysis of FGFR3-expressing target cells was demonstrated in vitro in a calcein-release assay. The Natural Killer (NK) cell line, NKL, was co-cultured with calcein-loaded P815 target cells ectopically expressing FGFR3. The results in FIG. 15 showed that the two MICA-α3-iFv molecules induced significantly greater NK-mediated lysis compared to the traditional MICA-scFv fusion, while the non-targeted soluble MICA control had no killing activity. These results confirmed that the invented MICA-α3-iFv bound FGFR3 on target cells and induced potent NK cell-mediated lysis.

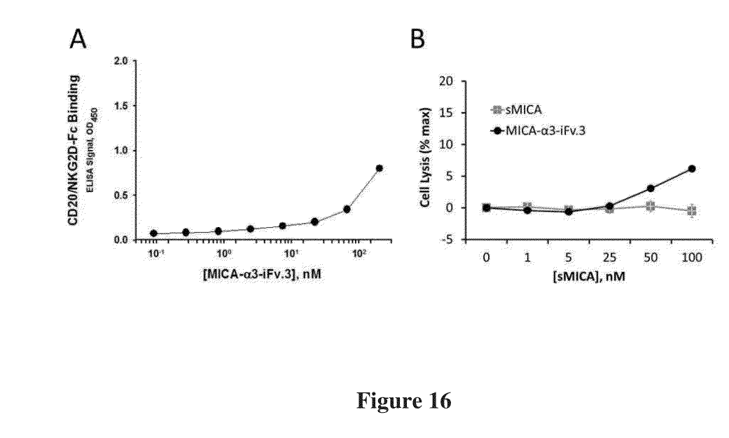

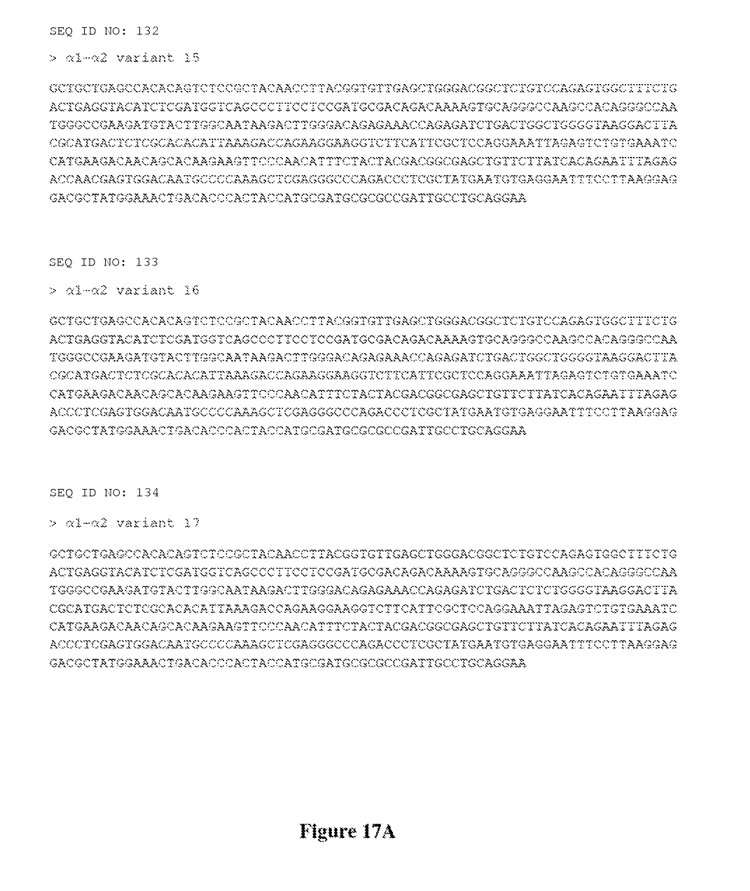

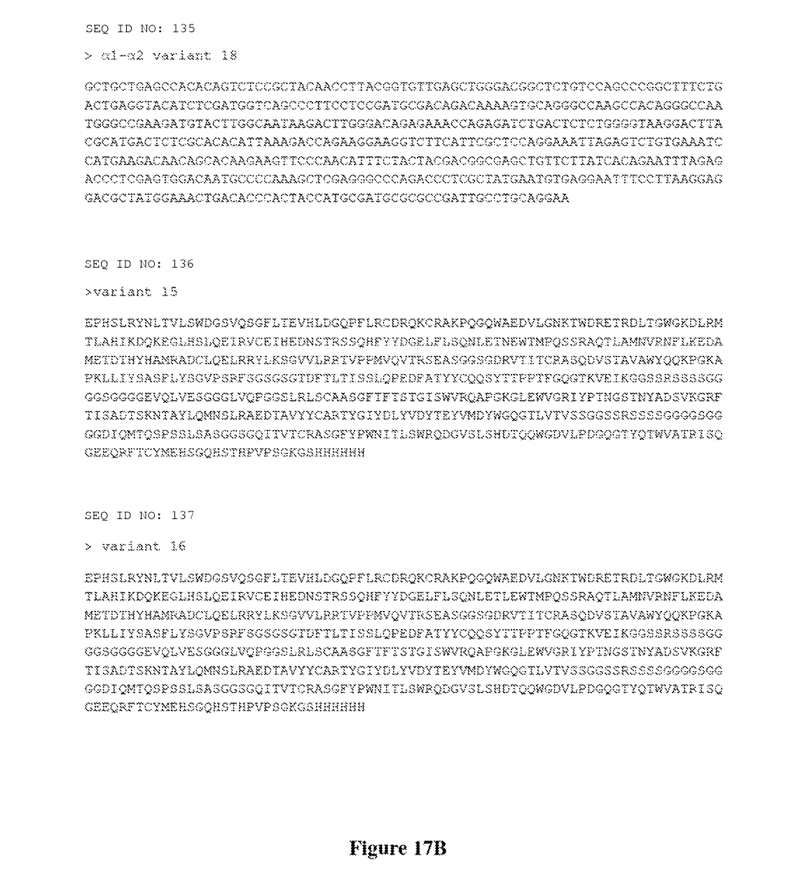

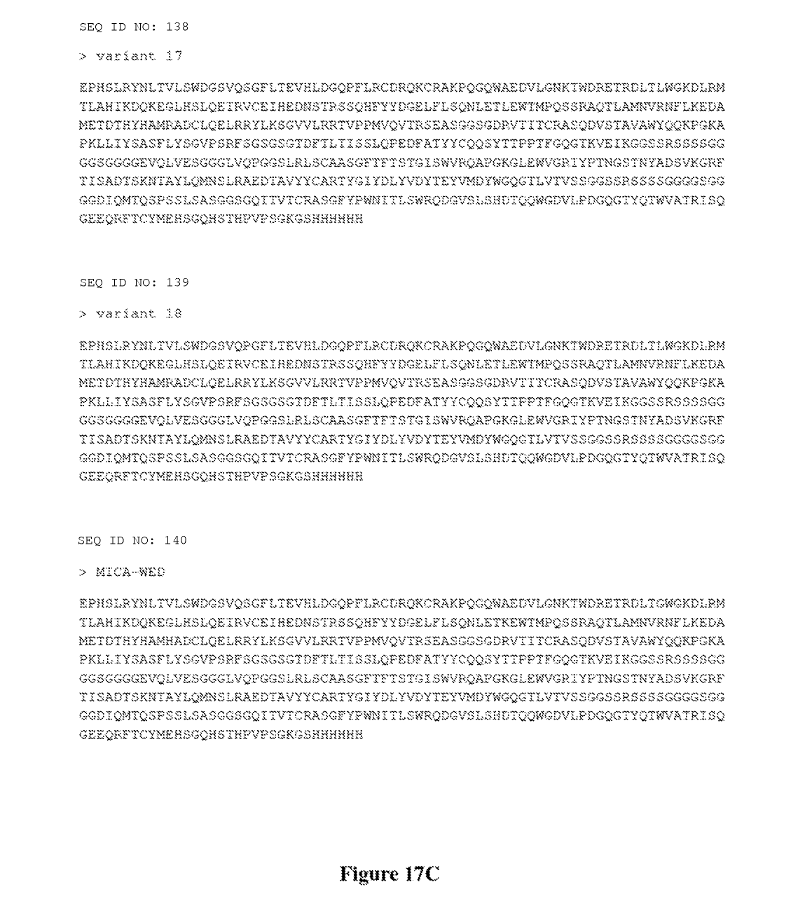

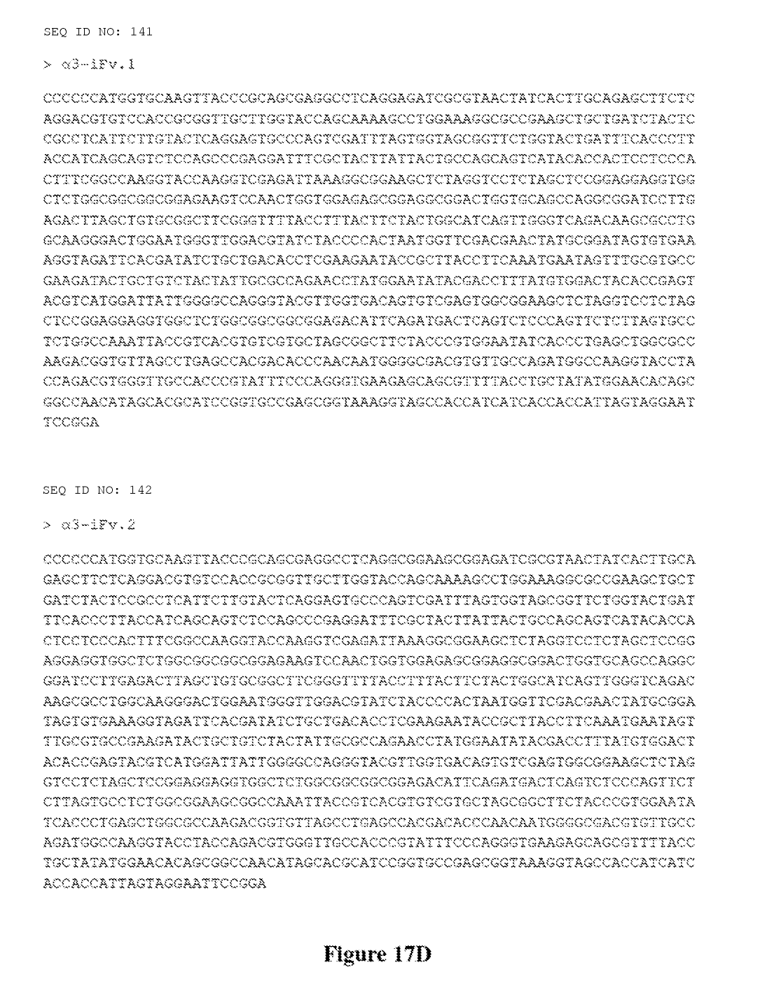

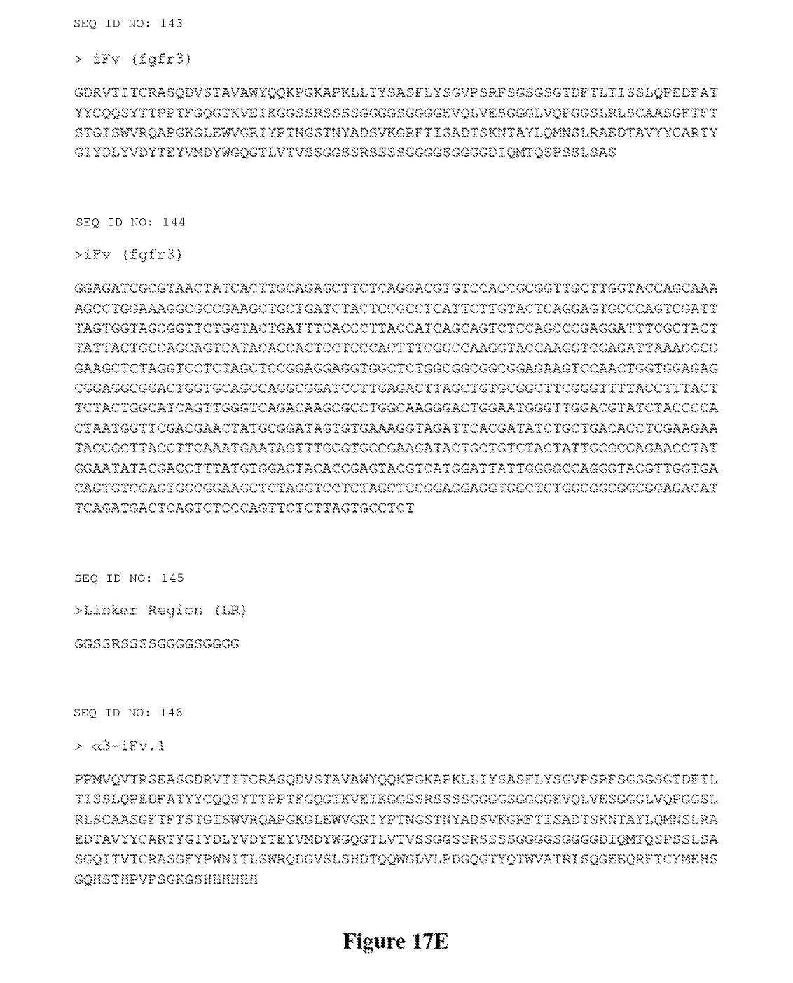

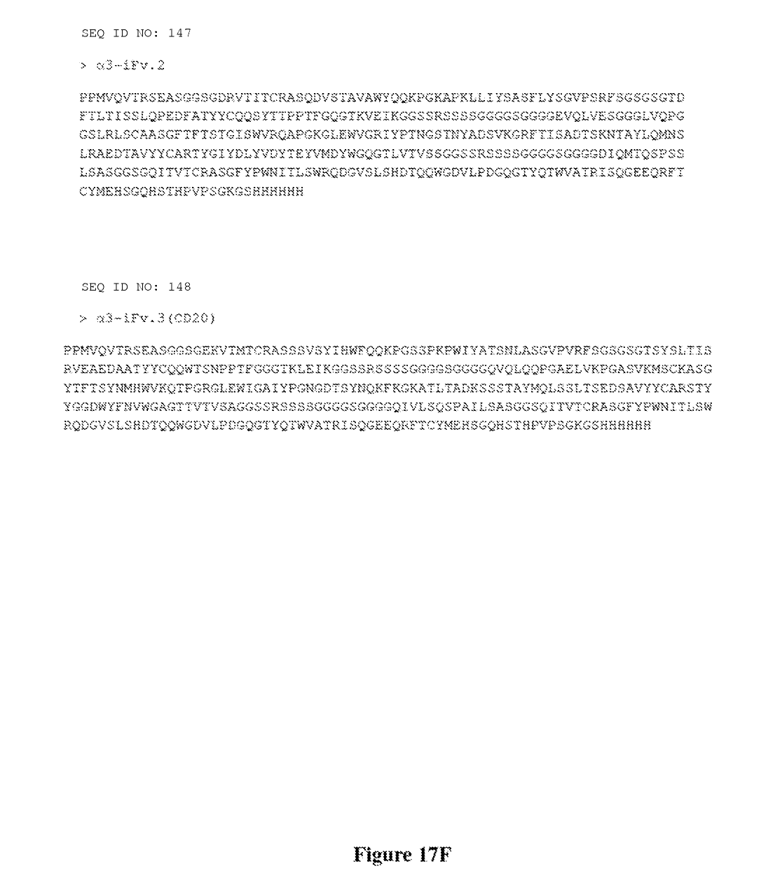

The applicability of the iFv format to other antibody variable domains was demonstrated by similarly constructing an α3-iFv.3 (SEQ ID NO.:143), which contained an iFv derived from a CD20-specific antibody (Du, J., Wang, H., Zhong, C., Peng, B., Zhang, M., Li, B., Huo, S., Guo, Y., and Ding, J. (2007) Structural basis for recognition of CD20 by therapeutic antibody Rituximab, The Journal of biological chemistry 282, 15073-15080). FIG. 16 showed that MICA-α3-iFv.3 was able to specifically bind wells coated with CD20 in a plate-based ELISA as described above and also induced NK-mediated lysis of Ramos cells expressing CD20 in a calcein-release assay.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

All references cited herein are hereby incorporated by reference in their entireties, whether previously specifically incorporated or not. As used herein, the terms “a”, “an”, and “any” are each intended to include both the singular and plural forms.

Having now fully described the invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and may be applied to the essential features hereinbefore set forth.

1. A non-natural, monomeric, soluble, mammalian MHC class I chain-related (MIC) molecule comprising a modified α1-α2 platform domain attached to a targeting motif,
wherein the modified α1-α2 platform domain is at least 80% identical to a native α1-α2 platform domain of a MIC protein, and wherein the α1-α2 platform domain has been modified to alter its binding affinity to a human NKG2D, and
wherein the targeting motif comprises a MIC α3 domain and one or more heterologous peptides, wherein the heterologous peptide or peptides are inserted into the MIC α3 domain within one or more sites in a solvent-exposed loop at a non-carboxy-terminal site, and
wherein the heterologous peptide or peptides direct the binding of the targeting motif to one or more target molecules on one or more target cells, thereby delivering the attached modified α1-α2 platform domain to the target cell.

2. The MIC molecule of claim 1 which exhibits a greater affinity binding to the NKG2D as compared to a MIC protein selected from the group consisting of SEQ ID NOs: 1-13 and 140.

3. The MIC molecule of claim 2 which exhibits an enhanced activation of a cell expressing NKG2D, resulting in the cell having a greater target cell killing potency.

4. The MIC molecule of claim 1 wherein the modified α1-α2 platform domain comprises an amino acid replaced at position 20, 68, 125, 152, 161, or 166, or at a combination of positions thereof, based on SEQ ID NO.: 140.

5. The MIC molecule of claim 1 wherein its altered binding affinity to NKG2D is effected by an altered off-rate.

6. The MIC molecule of claim 2 wherein its greater binding affinity to NKG2D is effected by a slower off-rate.

7. The MIC molecule of claim 6
wherein the amino acid at position 20 is P, T, D, A, L or N;
wherein the amino acid at position 68 is L, F, S, A, Y, I, E, T or W;
wherein the amino acid at position 125 is L, R, F, T, A, N, V, Y, I, or S;
wherein the amino acid at position 152 is E, T, V, G, F, Y, A, Q, D, I, N, S, H, M, or P;
wherein the amino acid at position 161 is R, S, A, K, G, L, F, or Y; or
wherein the amino acid at position 166 is F, S, H, Y, W, V, L, or M; or combinations of such positional changes thereof.

8. The MIC molecule of claim 7 comprising SEQ ID NO: 136, SEQ ID NO: 137, SEQ ID NO: 138, or SEQ ID NO: 139.

9. The MIC molecule of claim 2 which exhibits an affinity for a murine NKG2D greater than the MIC protein selected from the group consisting of SEQ ID. NOs.: 1-13 and 140.

10. The MIC molecule of claim 1 wherein the heterologous peptide or peptides are comprised of an insertable variable fragment of an antibody (iFv).

11. The MIC molecule of claim 10 wherein at least one of the target molecules is FGFR3.

12. The MIC molecule of claim 10 wherein at least one of the target molecules is CD20.

13. A composition comprising the MIC molecule of claim 1 and a carrier or excipient.

14. A nucleic acid molecule encoding the MIC molecule of claim 1.

15. An expression cassette comprising the nucleic acid molecule of claim 14.

16. A method of treating a mammal suspected of having a malignancy or viral infection comprising administering an effective amount of the MIC molecule of claim 1 to said mammal, wherein the target cell is a malignant cell or a virus-infected cell.

17. The method of claim 16 wherein the administered MIC molecule binds a NKG2D-bearing cell and the malignant cell or the virus-infected cell, resulting in the adhesion of the NKG2D-bearing cell to the malignant cell or to the virus-infected cell.

18. The method of claim 17 wherein the adhering NKG2D-bearing cell destroys the viability of the malignant cell or of the virus-infected cell.

19. The molecule of claim 2, wherein all or a portion of one or more of the solvent-exposed loops is deleted and replaced with the heterologous peptide or peptides.

20. The molecule of claim 19, wherein all of one or more of the solvent-exposed loops is deleted, and wherein further one, two, three, four, or five additional amino acids of the α3 domain adjacent to one or both sides of the deleted loop are deleted.


SEQUENCE LISTING (148 sequences)

SEQ ID NO: 1 - protein, 274 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTLYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 2 - protein, 274 aa, Homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNIRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 3 - protein, 274 aa, Homo sapiens
EPHSLPYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 4 - protein, 274 aa, Homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 5 - protein, 274 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 6 - protein, 274 aa, Homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 7 - protein, 305 aa, Homo sapiens
PHSLRYNLMVLSQDGSVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQLPPMVNVICSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGP

SEQ ID NO: 8 - protein, 317 aa, Homo sapiens
PHSLRYNLMVLSQDGSVQSGFLAEGHLDGQPFLRYDRQKRRAKPQGQWAEDVLGAETWDTETEDLTENGQDLRRTLTHIKDQKGGLHSLQEIRVCEMHEDSSTRGSRHFYYNGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGP

SEQ ID NO: 9 - protein, 317 aa, Homo sapiens
PHSLRYNLMVLSQDGSVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVICSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGP

SEQ ID NO: 10 - protein, 317 aa, Homo sapiens
PHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGP

SEQ ID NO: 11 - protein, 317 aa, Homo sapiens
PHSLRYNLMVLSQDGSVQSGFLAEGHLDGQPFLRYDRQKRRAKPQGQWAEDVLGAETWDTETEDLTENGQDLRRTLTHIKDQKGGLHSLQEIRVCEIHEDSSTRGSRHFYYNGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQKFTCYMEHSGNHGTHPVPSGKALVLQSQRTDFPYVSAAMPCFVIIIILCVPCCKKKTSAAEGP

SEQ ID NO: 12 - protein, 317 aa, Homo sapiens
PHSLRYNLMVLSQDGSVQSGFLAEGHLDGQPFLRYDRQKRRAKPQGQWAEDVLGAETWDTETEDLTENGQDLRRTLTHIKDQKGGLHSLQEIRVCEIHEDSSTRGSRHFYYNGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGP

SEQ ID NO: 13 - protein, 276 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGK

SEQ ID NO: 14 - DNA, 937 bp, Homo sapiens
gctagcgctgagagggtggcgacgtcggggccatggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaataaaagctt

SEQ ID NO: 15 - DNA, 57 bp, Artificial Sequence
  Description of Artificial Sequence Synthetic oligonucleotide
tatgaaatacctgctgccgaccgctgctgctggtctgctgctcctcgctgcccagcc

SEQ ID NO: 16 - DNA, 59 bp, Artificial Sequence
  Description of Artificial Sequence Synthetic oligonucleotide
catgggctgggcagcgaggagcagcagaccagcagcagcggtcggcagcaggtatttca

SEQ ID NO: 17 - DNA, 48 bp, Artificial Sequence
  Description of Artificial Sequence Synthetic oligonucleotide
catgcatcatcaccatcaccacctcgaggaattcaagcttggatccgc

SEQ ID NO: 18 - DNA, 47 bp, Artificial Sequence
  Description of Artificial Sequence Synthetic oligonucleotide
tcagcggatccaagcttgaattcctcgaggtggtgatggtgatgatg

SEQ ID NO: 19 - DNA, 32 bp, Artificial Sequence
  Description of Artificial Sequence Synthetic primer
ttttttgctagcgctgagagggtggcgacgtc

SEQ ID NO: 20 - DNA, 36 bp, Artificial Sequence
  Description of Artificial Sequence Synthetic primer
ctttccaagcttttatttcccagagggcacagggtg

SEQ ID NO: 21 - DNA, 58 bp, Artificial Sequence
  Description of Artificial Sequence Synthetic primer
tccctcctcgaggaaaacttgtactttcaaggcgagccccacagtcttcgttataacc

SEQ ID NO: 22 - DNA, 38 bp, Artificial Sequence
  Description of Artificial Sequence Synthetic primer
ccccccggatccatgattactcatggttgttatacccg

SEQ ID NO: 23 - DNA, 34 bp, Artificial Sequence
  Description of Artificial Sequence Synthetic primer
ccccccaagcttattctacacaaaccgcatagac

SEQ ID NO: 24 - DNA, 66 bp, Artificial Sequence
  Description of Artificial Sequence Synthetic primer
ttttttctcgaggtggtgatggtgatgatgtcggccttcaataccgccgctggccttggtttgatc

SEQ ID NO: 25 - DNA, 36 bp, Artificial Sequence
  Description of Artificial Sequence Synthetic primer
cccccccatatgattactcatggttgttatacccgg

SEQ ID NO: 26 - DNA, 65 bp, Artificial Sequence
  Description of Artificial Sequence Synthetic primer
aaaaaactcgaggaaaacttgtactttcaaggcacagtgccacccatggtgaatgtcacccgcag

SEQ ID NO: 27 - DNA, 30 bp, Artificial Sequence
  Description of Artificial Sequence Synthetic primer
atatataagcttttatttcccagagggcac

SEQ ID NO: 28 - DNA, 29 bp, Artificial Sequence
  Description of Artificial Sequence Synthetic primer
ttttttcgtctctcatgattactcatggt

SEQ ID NO: 29 - DNA, 48 bp, Artificial Sequence
  Description of Artificial Sequence Synthetic primer
atatacatacagtcgaccaggttgggggcggtattgaaggccgacatc

SEQ ID NO: 30 - DNA, 32 bp, Artificial Sequence; identical to SEQ ID NO: 19
  Description of Artificial Sequence Synthetic primer

SEQ ID NO: 31 - DNA, 36 bp, Artificial Sequence; identical to SEQ ID NO: 20
  Description of Artificial Sequence Synthetic primer

SEQ ID NO: 32 - DNA, 24 bp, Artificial Sequence
  Description of Artificial Sequence Synthetic primer
aatcacagcactcaccctgtgccc

SEQ ID NO: 33 - DNA, 42 bp, Artificial Sequence
  Description of Artificial Sequence Synthetic primer
tcccttcgtctctggtcggatacgctgtcgaacttttcgatc

SEQ ID NO: 34 - DNA, 26 bp, Artificial Sequence
  Description of Artificial Sequence Synthetic primer
gaatcctggtggccacccaggtctgg

SEQ ID NO: 35 - DNA, 43 bp, Artificial Sequence
  Description of Artificial Sequence Synthetic primer
gagacgacaaacgtctcttgctacatggaacacagcgggaatc

SEQ ID NO: 36 - DNA, 79 bp, Artificial Sequence
  Description of Artificial Sequence Synthetic oligonucleotide
gattagtggtggcagtggcggcggtagtcatcatcaccaccatcaccaccatcaccacagcggcggcagcggtggcggt

SEQ ID NO: 37 - DNA, 79 bp, Artificial Sequence
  Description of Artificial Sequence Synthetic oligonucleotide
agcaaccgccaccgctgccgccgctgtggtgatggtggtgatggtggtgatgatgactaccgccgccactgccaccact

SEQ ID NO: 38 - protein, 25 aa, Artificial Sequence
  Description of Artificial Sequence Synthetic peptide
SGGSGGGSHHHHHHHHHHSGGSGGG

SEQ ID NO: 39 - DNA, 30 bp, Artificial Sequence
  Description of Artificial Sequence Synthetic primer
agtcagggtttctcactgaggtacatctgg

SEQ ID NO: 40 - DNA, 31 bp, Artificial Sequence
  Description of Artificial Sequence Synthetic primer
gcacagatccatcccaggacagcaccgtgag

SEQ ID NO: 41 - DNA, 34 bp, Artificial Sequence
  Description of Artificial Sequence Synthetic primer
catcatcatgagccccacagtcttcgttataacc

SEQ ID NO: 42 - DNA, 37 bp, Artificial Sequence
  Description of Artificial Sequence Synthetic primer
gtggtggtgagcagcagctcccggaggtgcaaaaggg

SEQ ID NO: 43 - protein, 9 aa, Artificial Sequence
  Description of Artificial Sequence Synthetic peptide
SRGDHPRTQ

SEQ ID NO: 44 - DNA, 31 bp, Artificial Sequence
  Description of Artificial Sequence Synthetic oligonucleotide
cacctctcggggcgatcaccctcgcacccag

SEQ ID NO: 45 - DNA, 31 bp, Artificial Sequence
  Description of Artificial Sequence Synthetic oligonucleotide
tcacctgggtgcgagggtgatcgccccgaga

SEQ ID NO: 46 - protein, 11 aa, Artificial Sequence
  Description of Artificial Sequence Synthetic peptide
RTSRGDHPRTQ

SEQ ID NO: 47 - DNA, 37 bp, Artificial Sequence
  Description of Artificial Sequence Synthetic oligonucleotide
caccaggacatctcggggcgatcaccctcgcacccag

SEQ ID NO: 48 - DNA, 37 bp, Artificial Sequence
  Description of Artificial Sequence Synthetic oligonucleotide
tcacctgggtgcgagggtgatcgccccgagatgtcct

SEQ ID NO: 49 - protein, 10 aa, Artificial Sequence
  Description of Artificial Sequence Synthetic peptide
RVPRGDSDLT

SEQ ID NO: 50 - DNA, 34 bp, Artificial Sequence
  Description of Artificial Sequence Synthetic oligonucleotide
caccagggtgcctcggggcgatagcgatctgacc

SEQ ID NO: 51 - DNA, 34 bp, Artificial Sequence
  Description of Artificial Sequence Synthetic oligonucleotide
tcacggtcagatcgctatcgccccgaggcaccct

SEQ ID NO: 52 - protein, 10 aa, Artificial Sequence
  Description of Artificial Sequence Synthetic peptide
RSARGDSDHR

SEQ ID NO: 53 - DNA, 34 bp, Artificial Sequence
  Description of Artificial Sequence Synthetic oligonucleotide
caccaggagcgcccggggcgatagcgatcaccgg

SEQ ID NO: 54 - DNA, 34 bp, Artificial Sequence
  Description of Artificial Sequence Synthetic oligonucleotide
tcacccggtgatcgctatcgccccgggcgctcct

SEQ ID NO: 55 - protein, 10 aa, Artificial Sequence
  Description of Artificial Sequence Synthetic peptide
VTRGDTFTQS

SEQ ID NO: 56 - DNA, 34 bp, Artificial Sequence
  Description of Artificial Sequence Synthetic oligonucleotide
caccgtgacacggggcgatactttcacacagtcc

SEQ ID NO: 57 - DNA, 34 bp, Artificial Sequence
  Description of Artificial Sequence Synthetic oligonucleotide
tcacggactgtgtgaaagtatcgccccgtgtcac

SEQ ID NO: 58 - protein, 8 aa, Artificial Sequence
  Description of Artificial Sequence Synthetic peptide
RGDTFTQS

SEQ ID NO: 59 - DNA, 28 bp, Artificial Sequence
  Description of Artificial Sequence Synthetic oligonucleotide
cacccggggcgatactttcacacagtcc

SEQ ID NO: 60 - DNA, 28 bp, Artificial Sequence
  Description of Artificial Sequence Synthetic oligonucleotide
tcacggactgtgtgaaagtatcgccccg

SEQ ID NO: 61 - protein, 10 aa, Artificial Sequence
  Description of Artificial Sequence Synthetic peptide
HLARGDDLTY

SEQ ID NO: 62 - DNA, 34 bp, Artificial Sequence
  Description of Artificial Sequence Synthetic oligonucleotide
cacccacctggcacggggcgatgacctgacatac

SEQ ID NO: 63 - DNA, 34 bp, Artificial Sequence
  Description of Artificial Sequence Synthetic oligonucleotide
tcacgtatgtcaggtcatcgccccgtgccaggtg

SEQ ID NO: 64 - protein, 25 aa, Artificial Sequence
  Description of Artificial Sequence Synthetic peptide
SGGSGGGSTSRGDHPRTQSGGSGGG

SEQ ID NO: 65 - DNA, 79 bp, Artificial Sequence
  Description of Artificial Sequence Synthetic oligonucleotide
caccagtggtggcagtggcggcggtagtacatctcggggcgatcaccctcgcacccagagcggcggcagcggtggcggt

SEQ ID NO: 66 - DNA, 79 bp, Artificial Sequence
  Description of Artificial Sequence Synthetic oligonucleotide
tcacaccgccaccgctgccgccgctctgggtgcgagggtgatcgccccgagatgtactaccgccgccactgccaccact

SEQ ID NO: 67 - protein, 25 aa, Artificial Sequence
  Description of Artificial Sequence Synthetic peptide
SGGSGGGSRVPRGDSDLTSGGSGGG

SEQ ID NO: 68 - DNA, 79 bp, Artificial Sequence
  Description of Artificial Sequence Synthetic oligonucleotide
caccagtggtggcagtggcggcggtagtagggtgcctcggggcgatagcgatctgaccagcggcggcagcggtggcggt

SEQ ID NO: 69 - DNA, 79 bp, Artificial Sequence
  Description of Artificial Sequence Synthetic oligonucleotide
tcacaccgccaccgctgccgccgctggtcagatcgctatcgccccgaggcaccctactaccgccgccactgccaccact

SEQ ID NO: 70 - protein, 25 aa, Artificial Sequence
  Description of Artificial Sequence Synthetic peptide
SGGSGGGSVTRGDTFTQSSGGSGGG

SEQ ID NO: 71 - DNA, 79 bp, Artificial Sequence
  Description of Artificial Sequence Synthetic oligonucleotide
caccagtggtggcagtggcggcggtagtgtgacacggggcgatactttcacacagtccagcggcggcagcggtggcggt

SEQ ID NO: 72 - DNA, 79 bp, Artificial Sequence
  Description of Artificial Sequence Synthetic oligonucleotide
tcacaccgccaccgctgccgccgctggactgtgtgaaagtatcgccccgtgtcacactaccgccgccactgccaccact

SEQ ID NO: 73 - protein, 25 aa, Artificial Sequence
  Description of Artificial Sequence Synthetic peptide
SGGSGGGSHLARGDDLTYSGGSGGG

SEQ ID NO: 74 - DNA, 79 bp, Artificial Sequence
  Description of Artificial Sequence Synthetic oligonucleotide
caccagtggtggcagtggcggcggtagtcacctggcacggggcgatgacctgacatacagcggcggcagcggtggcggt

SEQ ID NO: 75 - DNA, 79 bp, Artificial Sequence
  Description of Artificial Sequence Synthetic oligonucleotide
tcacaccgccaccgctgccgccgctgtatgtcaggtcatcgccccgtgccaggtgactaccgccgccactgccaccact

SEQ ID NO: 76 - protein, 25 aa, Artificial Sequence
  Description of Artificial Sequence Synthetic peptide
SGGSGGGSTSRGDHPRTQSGGSGGG

SEQ ID NO: 77 - DNA, 79 bp, Artificial Sequence
  Description of Artificial Sequence Synthetic oligonucleotide
gattagtggtggcagtggcggcggtagtacatctcggggcgatcaccctcgcacccagagcggcggcagcggtggcggt

SEQ ID NO: 78 - DNA, 79 bp, Artificial Sequence
  Description of Artificial Sequence Synthetic oligonucleotide
agcaaccgccaccgctgccgccgctctgggtgcgagggtgatcgccccgagatgtactaccgccgccactgccaccact

SEQ ID NO: 79 - protein, 25 aa, Artificial Sequence
  Description of Artificial Sequence Synthetic peptide
SGGSGGGSRVPRGDSDLTSGGSGGG

SEQ ID NO: 80 - DNA, 79 bp, Artificial Sequence
  Description of Artificial Sequence Synthetic oligonucleotide
gattagtggtggcagtggcggcggtagtagggtgcctcggggcgatagcgatctgaccagcggcggcagcggtggcggt

SEQ ID NO: 81 - DNA, 79 bp, Artificial Sequence
  Description of Artificial Sequence Synthetic oligonucleotide
agcaaccgccaccgctgccgccgctggtcagatcgctatcgccccgaggcaccctactaccgccgccactgccaccact

SEQ ID NO: 82 - protein, 25 aa, Artificial Sequence
  Description of Artificial Sequence Synthetic peptide
SGGSGGGSVTRGDTFTQSSGGSGGG

SEQ ID NO: 83 - DNA, 79 bp, Artificial Sequence
  Description of Artificial Sequence Synthetic oligonucleotide
gattagtggtggcagtggcggcggtagtgtgacacggggcgatactttcacacagtccagcggcggcagcggtggcggt

SEQ ID NO: 84 - DNA, 79 bp, Artificial Sequence
  Description of Artificial Sequence Synthetic oligonucleotide
agcaaccgccaccgctgccgccgctggactgtgtgaaagtatcgccccgtgtcacactaccgccgccactgccaccact

SEQ ID NO: 85 - protein, 25 aa, Artificial Sequence
  Description of Artificial Sequence Synthetic peptide
SGGSGGGSHLARGDDLTYSGGSGGG

SEQ ID NO: 86 - DNA, 79 bp, Artificial Sequence
  Description of Artificial Sequence Synthetic oligonucleotide
gattagtggtggcagtggcggcggtagtcacctggcacggggcgatgacctgacatacagcggcggcagcggtggcggt

SEQ ID NO: 87 - DNA, 79 bp, Artificial Sequence
  Description of Artificial Sequence Synthetic oligonucleotide
agcaaccgccaccgctgccgccgctgtatgtcaggtcatcgccccgtgccaggtgactaccgccgccactgccaccact

SEQ ID NO: 88 - DNA, 41 bp, Artificial Sequence
  Description of Artificial Sequence Synthetic primer
ttaattaacgtctcatgcagggcttctggcttctatccctg

SEQ ID NO: 89 - DNA, 36 bp, Artificial Sequence
  Description of Artificial Sequence Synthetic primer
acgtctcgattcaccatggggggcactgttctcctc

SEQ ID NO: 90 - protein, 10 aa, Artificial Sequence
  Description of Artificial Sequence Synthetic peptide
TSRGDHPRTQ

SEQ ID NO: 91 - DNA, 34 bp, Artificial Sequence
  Description of Artificial Sequence Synthetic oligonucleotide
gaatacaagccgaggtgaccacccacgtacacaa

SEQ ID NO: 92 - DNA, 34 bp, Artificial Sequence
  Description of Artificial Sequence Synthetic oligonucleotide
tgcattgtgtacgtgggtggtcacctcggcttgt

SEQ ID NO: 93 - protein, 10 aa, Artificial Sequence
  Description of Artificial Sequence Synthetic peptide
GSRGDSLIMH

SEQ ID NO: 94 - DNA, 34 bp, Artificial Sequence
  Description of Artificial Sequence Synthetic oligonucleotide
gaatggttcacgaggtgactcattgattatgcac

SEQ ID NO: 95 - DNA, 34 bp, Artificial Sequence
  Description of Artificial Sequence Synthetic oligonucleotide
tgcagtgcataatcaatgagtcacctcgtgaacc

SEQ ID NO: 96 - protein, 10 aa, Artificial Sequence
  Description of Artificial Sequence Synthetic peptide
RVPRGDSDLT

SEQ ID NO: 97 - DNA, 34 bp, Artificial Sequence
  Description of Artificial Sequence Synthetic oligonucleotide
gaatcgagtaccacgaggtgactcagatttgact

SEQ ID NO: 98 - DNA, 34 bp, Artificial Sequence
  Description of Artificial Sequence Synthetic oligonucleotide
tgcaagtcaaatctgagtcacctcgtggtactcg

SEQ ID NO: 99 - protein, 10 aa, Artificial Sequence
  Description of Artificial Sequence Synthetic peptide
VTRGDTFTQS

SEQ ID NO: 100 - DNA, 34 bp, Artificial Sequence
  Description of Artificial Sequence Synthetic oligonucleotide
gaatgtaacacgaggtgacacattcactcagagc

SEQ ID NO: 101 - DNA, 34 bp, Artificial Sequence
  Description of Artificial Sequence Synthetic oligonucleotide
tgcagctctgagtgaatgtgtcacctcgtgttac

SEQ ID NO: 102 - protein, 10 aa, Artificial Sequence
  Description of Artificial Sequence Synthetic peptide
HLARGDDLTY

SEQ ID NO: 103 - DNA, 34 bp, Artificial Sequence
  Description of Artificial Sequence Synthetic oligonucleotide
gaatcacttggcacgaggtgacgatctcacatac

SEQ ID NO: 104 - DNA, 34 bp, Artificial Sequence
  Description of Artificial Sequence Synthetic oligonucleotide
tgcagtatgtgagatcgtcacctcgtgccaagtg

SEQ ID NO: 105 - protein, 8 aa, Artificial Sequence
  Description of Artificial Sequence Synthetic peptide
YQSWRYSQ

SEQ ID NO: 106 - DNA, 28 bp, Artificial Sequence
  Description of Artificial Sequence Synthetic oligonucleotide
gaattaccagtcttggcgttactctcag

SEQ ID NO: 107 - DNA, 28 bp, Artificial Sequence
  Description of Artificial Sequence Synthetic oligonucleotide
tgcactgagagtaacgccaagactggta

SEQ ID NO: 108 - DNA, 26 bp, Artificial Sequence
  Description of Artificial Sequence Synthetic primer
gaatcctggtggccacccaggtctgg

SEQ ID NO: 109 - DNA, 43 bp, Artificial Sequence; identical to SEQ ID NO: 35
  Description of Artificial Sequence Synthetic primer

SEQ ID NO: 110 - DNA, 79 bp, Artificial Sequence
  Description of Artificial Sequence Synthetic oligonucleotide
gatttccggaggttctggaggtggctcggtaacccgaggagacacctttacccaaagttcaggaggttcaggaggtgga

SEQ ID NO: 111 - DNA, 79 bp, Artificial Sequence
  Description of Artificial Sequence Synthetic oligonucleotide
agcatccacctcctgaacctcctgaactttgggtaaaggtgtctcctcgggttaccgagccacctccagaacctccgga

SEQ ID NO: 112 - DNA, 53 bp, Artificial Sequence
  Description of Artificial Sequence Synthetic primer
cctccgaattcggatcctaggcggctccttatttgtttgtgaatatcaaggcc

SEQ ID NO: 113 - DNA, 35 bp, Artificial Sequence
  Description of Artificial Sequence Synthetic primer
ccctccaagcttaagactccttattacgcagtatg

SEQ ID NO: 114 - DNA, 299 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctgtaccagtcttggcgttactctcaggctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 115 - DNA, 305 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctgacatcacgaggcgaccacccacgcacccaggctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 116 - DNA, 305 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctgggctcacgaggcgactccctcatcatgcacgctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 117 - DNA, 305 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctggtaacacgaggcgacaccttcacgcagtccgctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 118 - DNA, 305 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctgcacctggcacgaggcgacgatcttacctacgctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 119 - DNA, 305 bp, Artificial Sequence
  Description of Artificial Sequence Synthetic polynucleotide
gaattcatgaaaaaattattattcgcaattcctttagtggtacctttctattctcactcggactacaaggatgacgacgataagcaattggaaccagcgccatcttgcgttaccctgcaccaccaccatcaccatcatcattcacaagctgacaacggttgcgcagaaacggttaccgtaaaagtggtatacgaagacgacaccgagggcctgtgctacgcagttgccccgggtcagatcaccactgttggtgacggctacatcggctctcacggtcacgctcggtatctggctcgttgcctagg

SEQ ID NO: 120 - DNA, 41 bp, Artificial Sequence
  Description of Artificial Sequence Synthetic primer
cttcccgaattcatgacagtgccacccatggtgaatgtcac

SEQ ID NO: 121 - DNA, 42 bp, Artificial Sequence
  Description of Artificial Sequence Synthetic primer
tttcttcgtctcactagtttcccagagggcacagggtgagtg

SEQ ID NO: 122 - DNA, 41 bp, Artificial Sequence
  Description of Artificial Sequence Synthetic primer
cttcccgaattcatgacagtgccccccatggtgaatacaag

SEQ ID NO: 123 - DNA, 42 bp, Artificial Sequence
  Description of Artificial Sequence Synthetic primer
cttcccgaattcatgacagtgccccccatggtgaatggttca

SEQ ID NO: 124 - DNA, 42 bp, Artificial Sequence
  Description of Artificial Sequence Synthetic primer
cttcccgaattcatgacagtgccccccatggtgaatcgagta

SEQ ID NO: 125 - DNA, 42 bp, Artificial Sequence
  Description of Artificial Sequence Synthetic primer
cttcccgaattcatgacagtgccccccatggtgaatgtaaca

SEQ ID NO: 126 - DNA, 42 bp, Artificial Sequence
  Description of Artificial Sequence Synthetic primer
cttcccgaattcatgacagtgccccccatggtgaatcacttg

SEQ ID NO: 127 - protein, 6 aa, Artificial Sequence
  Description of Artificial Sequence Synthetic 6xHis tag
HHHHHH

SEQ ID NO: 128 - protein, 7 aa, Unknown
  Description of Unknown Tev protease cleavage site peptide
ENLYFQG

SEQ ID NO: 129 - protein, 4 aa, Unknown
  Description of Unknown Factor Xa cleavage site peptide
IEGR

SEQ ID NO: 130 - protein, 8 aa, Artificial Sequence
  Description of Artificial Sequence Synthetic 8xHis tag
HHHHHHHH

SEQ ID NO: 131 - protein, 10 aa, Artificial Sequence
  Description of Artificial Sequence Synthetic 10xHis tag
HHHHHHHHHH

SEQ ID NO: 132 - DNA, 507 bp, Artificial Sequence
  Description of Artificial Sequence Synthetic polynucleotide
gctgctgagccacacagtctccgctacaaccttacggtgttgagctgggacggctctgtccagagtggctttctgactgaggtacatctcgatggtcagcccttcctccgatgcgacagacaaaagtgcagggccaagccacagggccaatgggccgaagatgtacttggcaataagacttgggacagagaaaccagagatctgactggctggggtaaggacttacgcatgactctcgcacacattaaagaccagaaggaaggtcttcattcgctccaggaaattagagtctgtgaaatccatgaagacaacagcacaagaagttcccaacatttctactacgacggcgagctgttcttatcacagaatttagagaccaacgagtggacaatgccccaaagctcgagggcccagaccctcgctatgaatgtgaggaatttccttaaggaggacgctatggaaactgacacccactaccatgcgatgcgcgccgattgcctgcaggaa

SEQ ID NO: 133 - DNA, 507 bp, Artificial Sequence
  Description of Artificial Sequence Synthetic polynucleotide
gctgctgagccacacagtctccgctacaaccttacggtgttgagctgggacggctctgtccagagtggctttctgactgaggtacatctcgatggtcagcccttcctccgatgcgacagacaaaagtgcagggccaagccacagggccaatgggccgaagatgtacttggcaataagacttgggacagagaaaccagagatctgactggctggggtaaggacttacgcatgactctcgcacacattaaagaccagaaggaaggtcttcattcgctccaggaaattagagtctgtgaaatccatgaagacaacagcacaagaagttcccaacatttctactacgacggcgagctgttcttatcacagaatttagagaccctcgagtggacaatgccccaaagctcgagggcccagaccctcgctatgaatgtgaggaatttccttaaggaggacgctatggaaactgacacccactaccatgcgatgcgcgccgattgcctgcaggaa

SEQ ID NO: 134 - DNA, 507 bp, Artificial Sequence
  Description of Artificial Sequence Synthetic polynucleotide
gctgctgagccacacagtctccgctacaaccttacggtgttgagctgggacggctctgtccagagtggctttctgactgaggtacatctcgatggtcagcccttcctccgatgcgacagacaaaagtgcagggccaagccacagggccaatgggccgaagatgtacttggcaataagacttgggacagagaaaccagagatctgactctctggggtaaggacttacgcatgactctcgcacacattaaagaccagaaggaaggtcttcattcgctccaggaaattagagtctgtgaaatccatgaagacaacagcacaagaagttcccaacatttctactacgacggcgagctgttcttatcacagaatttagagaccctcgagtggacaatgccccaaagctcgagggcccagaccctcgctatgaatgtgaggaatttccttaaggaggacgctatggaaactgacacccactaccatgcgatgcgcgccgattgcctgcaggaa

SEQ ID NO: 135 - DNA, 507 bp, Artificial Sequence
  Description of Artificial Sequence Synthetic polynucleotide
gctgctgagccacacagtctccgctacaaccttacggtgttgagctgggacggctctgtccagcccggctttctgactgaggtacatctcgatggtcagcccttcctccgatgcgacagacaaaagtgcagggccaagccacagggccaatgggccgaagatgtacttggcaataagacttgggacagagaaaccagagatctgactctctggggtaaggacttacgcatgactctcgcacacattaaagaccagaaggaaggtcttcattcgctccaggaaattagagtctgtgaaatccatgaagacaacagcacaagaagttcccaacatttctactacgacggcgagctgttcttatcacagaatttagagaccctcgagtggacaatgccccaaagctcgagggcccagaccctcgctatgaatgtgaggaatttccttaaggaggacgctatggaaactgacacccactaccatgcgatgcgcgccgattgcctgcaggaa

SEQ ID NO: 136 - protein, 558 aa, Artificial Sequence
  Description of Artificial Sequence Synthetic polypeptide
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETNEWTMPQSSRAQTLAMNVRNFLKEDAMETDTHYHAMRADCLQELRRYLKSGVVLRRTVPPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 137 - protein, 558 aa, Artificial Sequence
  Description of Artificial Sequence Synthetic polypeptide
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLQELRRYLKSGVVLRRTVPPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 138 - protein, 558 aa, Artificial Sequence
  Description of Artificial Sequence Synthetic polypeptide
EPHSLRYNLTVLSWDGSVQSGFLTEVHLDGQPFLRCDRQKCRAKPQGQWAEDVLGNKTWDRETRDLTLWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLQELRRYLKSGVVLRRTVPPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 139 - protein, 558 aa, Artificial Sequence
  Description of Artificial Sequence Synthetic polypeptide
EPHSLRYNLTVLSWDGSVQPGFLTEVHLDGQPFLRCDRQKCRAKPQGQWAEDVLGNKTWDRETRDLTLWGKDLRMTLAHIKDQKEGLHSLQEIRVCEIHEDNSTRSSQHFYYDGELFLSQNLETLEWTMPQSSRAQTLAMNVRNFLKEDAMETDTHYHAMRADCLQELRRYLKSGVVLRRTVPPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 140 - protein, 558 aa, Artificial Sequence
  Description of Artificial Sequence Synthetic polypeptide
EPHSLRYNLTVLSWDGSVQSGFLTEVHLDGQPFLRCDRQKCRAKPQGQWAEDVLGNKTWDRETRDLTGWGKDLRMTLAHIKDQKEGLHSLQEIRVCEIHEDNSTRSSQHFYYDGELFLSQNLETKEWTMPQSSRAQTLAMNVRNFLKEDAMETDTHYHAMHADCLQELRRYLKSGVVLRRTVPPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 141 - DNA, 1126 bp, Artificial Sequence
  Description of Artificial Sequence Synthetic polynucleotide
ccccccatggtgcaagttacccgcagcgaggcctcaggagatcgcgtaactatcacttgcagagcttctcaggacgtgtccaccgcggttgcttggtaccagcaaaagcctggaaaggcgccgaagctgctgatctactccgcctcattcttgtactcaggagtgcccagtcgatttagtggtagcggttctggtactgatttcacccttaccatcagcagtctccagcccgaggatttcgctacttattactgccagcagtcatacaccactcctcccactttcggccaaggtaccaaggtcgagattaaaggcggaagctctaggtcctctagctccggaggaggtggctctggcggcggcggagaagtccaactggtggagagcggaggcggactggtgcagccaggcggatccttgagacttagctgtgcggcttcgggttttacctttacttctactggcatcagttgggtcagacaagcgcctggcaagggactggaatgggttggacgtatctaccccactaatggttcgacgaactatgcggatagtgtgaaaggtagattcacgatatctgctgacacctcgaagaataccgcttaccttcaaatgaatagtttgcgtgccgaagatactgctgtctactattgcgccagaacctatggaatatacgacctttatgtggactacaccgagtacgtcatggattattggggccagggtacgttggtgacagtgtcgagtggcggaagctctaggtcctctagctccggaggaggtggctctggcggcggcggagacattcagatgactcagtctcccagttctcttagtgcctctggccaaattaccgtcacgtgtcgtgctagcggcttctacccgtggaatatcaccctgagctggcgccaagacggtgttagcctgagccacgacacccaacaatggggcgacgtgttgccagatggccaaggtacctaccagacgtgggttgccacccgtatttcccagggtgaagagcagcgttttacctgctatatggaacacagcggccaacatagcacgcatccggtgccgagcggtaaaggtagccaccatcatcaccaccattagtaggaattccgga

SEQ ID NO: 142 - DNA, 1144 bp, Artificial Sequence
  Description of Artificial Sequence Synthetic polynucleotide
ccccccatggtgcaagttacccgcagcgaggcctcaggcggaagcggagatcgcgtaactatcacttgcagagcttctcaggacgtgtccaccgcggttgcttggtaccagcaaaagcctggaaaggcgccgaagctgctgatctactccgcctcattcttgtactcaggagtgcccagtcgatttagtggtagcggttctggtactgatttcacccttaccatcagcagtctccagcccgaggatttcgctacttattactgccagcagtcatacaccactcctcccactttcggccaaggtaccaaggtcgagattaaaggcggaagctctaggtcctctagctccggaggaggtggctctggcggcggcggagaagtccaactggtggagagcggaggcggactggtgcagccaggcggatccttgagacttagctgtgcggcttcgggttttacctttacttctactggcatcagttgggtcagacaagcgcctggcaagggactggaatgggttggacgtatctaccccactaatggttcgacgaactatgcggatagtgtgaaaggtagattcacgatatctgctgacacctcgaagaataccgcttaccttcaaatgaatagtttgcgtgccgaagatactgctgtctactattgcgccagaacctatggaatatacgacctttatgtggactacaccgagtacgtcatggattattggggccagggtacgttggtgacagtgtcgagtggcggaagctctaggtcctctagctccggaggaggtggctctggcggcggcggagacattcagatgactcagtctcccagttctcttagtgcctctggcggaagcggccaaattaccgtcacgtgtcgtgctagcggcttctacccgtggaatatcaccctgagctggcgccaagacggtgttagcctgagccacgacacccaacaatggggcgacgtgttgccagatggccaaggtacctaccagacgtgggttgccacccgtatttcccagggtgaagagcagcgttttacctgctatatggaacacagcggccaacatagcacgcatccggtgccgagcggtaaaggtagccaccatcatcaccaccattagtaggaattccgga

SEQ ID NO: 143 - protein, 269 aa, Artificial Sequence
  Description of Artificial Sequence Synthetic polypeptide
GDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSAS

SEQ ID NO: 144 - DNA, 807 bp, Artificial Sequence
  Description of Artificial Sequence Synthetic polynucleotide
ggagatcgcgtaactatcacttgcagagcttctcaggacgtgtccaccgcggttgcttggtaccagcaaaagcctggaaaggcgccgaagctgctgatctactccgcctcattcttgtactcaggagtgcccagtcgatttagtggtagcggttctggtactgatttcacccttaccatcagcagtctccagcccgaggatttcgctacttattactgccagcagtcatacaccactcctcccactttcggccaaggtaccaaggtcgagattaaaggcggaagctctaggtcctctagctccggaggaggtggctctggcggcggcggagaagtccaactggtggagagcggaggcggactggtgcagccaggcggatccttgagacttagctgtgcggcttcgggttttacctttacttctactggcatcagttgggtcagacaagcgcctggcaagggactggaatgggttggacgtatctaccccactaatggttcgacgaactatgcggatagtgtgaaaggtagattcacgatatctgctgacacctcgaagaataccgcttaccttcaaatgaatagtttgcgtgccgaagatactgctgtctactattgcgccagaacctatggaatatacgacctttatgtggactacaccgagtacgtcatggattattggggccagggtacgttggtgacagtgtcgagtggcggaagctctaggtcctctagctccggaggaggtggctctggcggcggcggagacattcagatgactcagtctcccagttctcttagtgcctct

SEQ ID NO: 145 - protein, 18 aa, Artificial Sequence
  Description of Artificial Sequence Synthetic peptide
GGSSRSSSSGGGGSGGGG

SEQ ID NO: 146 - protein, 370 aa, Artificial Sequence
  Description of Artificial Sequence Synthetic polypeptide
PPMVQVTRSEASGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 147 - protein, 376 aa, Artificial Sequence
  Description of Artificial Sequence Synthetic polypeptide
PPMVQVTRSEASGGSGDRVTITCRASQDVSTAVAWYQQKPGKAPKLLIYSASFLYSGVPSRFSGSGSGTDFTLTISSLQPEDFATYYCQQSYTTPPTFGQGTKVEIKGGSSRSSSSGGGGSGGGGEVQLVESGGGLVQPGGSLRLSCAASGFTFTSTGISWVRQAPGKGLEWVGRIYPTNGSTNYADSVKGRFTISADTSKNTAYLQMNSLRAEDTAVYYCARTYGIYDLYVDYTEYVMDYWGQGTLVTVSSGGSSRSSSSGGGGSGGGGDIQMTQSPSSLSASGGSGQITVTCRASGFYPWNITLSWRQDGVSLSHDTQQWGDVLPDGQGTYQTWVATRISQGEEQRFTCYMEHSGQHSTHPVPSGKGSHHHHHH

SEQ ID NO: 148 - protein, 368 aa, Artificial Sequence
  Description of Artificial Sequence Synthetic polypeptide
PPMVQVTRSEASGGSGEKVTMTCRASSSVSYIHWFQQKPGSSPKPWIYATSNLASGVPVRFSGSGSGTSYSLTISRVEAEDAATYYCQQWTSNPPTFGGGTKLEIKGGSSRSSSSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGSSRSSSSGGGGSGGGGQIVLSQSPAILSASGGSQITVTCRASGFYPWNITLSWRQDGVSLSHDTQQWGDVLPDGQGTYQTWVATRISQGEEQRFTCYMEHSGQHSTHPVPSGKGSHHHHHH